(19) United States
(12) Patent Application Publication
(10) Pub. No.: US 2023/0012148 A1
(43) Pub. Date: Jan. 12, 2023

(54) METHODS FOR TREATING PATIENTS WITH HEMATOLOGIC MALIGNANCIES

(71) Applicants: Aptos Biosciences Inc., Toronto (CA); CrystalGenomics Inc., Gyeonggi-do (KR)

(72) Inventors: William G. RICE, Del Mar, CA (US); Joong Myung CHO, Seoul (KR); Yongrae HONG, Gyeonggi-Do (KR)

(21) Appl. No.: 17/675,469

(22) Filed: Feb. 18, 2022

Related U.S. Application Data
(63) Continuation of application No. 15/901,439, filed on Feb. 21, 2018, now abandoned.
(60) Provisional application No. 62/578,948, filed on Oct. 30, 2017.
(60) Provisional application No. 62/461,584, filed on Feb. 21, 2017.

(51) Int. Cl. A61K 31/4178; A61P 35/02; C07D 403/04

(52) U.S. Cl. CPC A61K 31/4178; A61P 35/02; C07D 403/04

(57) ABSTRACT

The present disclosure comprises a method for administering 2,3-dihydro-isoindole-1-one compound or a pharmaceutically acceptable salt, ester, solvate and/or prodrug thereof, for the treatment of hematological cancers such as acute myeloid leukemia (AML). The present disclosure further relates to reducing or inhibiting cell-proliferation which is activated by wild-type or mutated Fms-like tyrosine kinase-3 receptor (FLT3). The present disclosure further relates to a method of inhibiting or reducing abnormal (e.g., overexpressed) wild-type or mutated BTK activity or expression in a subject in need thereof.

### CROSS-REFERENCE TO RELATED APPLICATIONS

This application is a continuation of U.S. Pat. Application No. 15/901,439, filed on Feb. 21, 2018, which claims priority to U.S. Provisional Application No. 62/461,584, filed on Feb. 21, 2017, and U.S. Provisional Application No. 62/578,948, filed on Oct. 30, 2017, the disclosures of which are hereby incorporated by reference in their entirety for all purposes.

### FIELD OF THE INVENTION

The present invention relates to a 2,3-dihydro-isoindole-1-one compound, or pharmaceutically acceptable salts, esters, prodrugs, hydrates, solvates and isomers thereof for the treatment of cancers, such as hematologic cancers, where the patients exhibit FLT3 mutations or wild type or mutant forms of BTK.

### BACKGROUND OF THE INVENTION

A number of tyrosine kinases have been shown to be part of regulatory pathways that promote cell-survival in many cancer types. The Fms-like tyrosine kinase 3 (FLT3) gene is one such example that encodes a membrane bound receptor tyrosine kinase that affects hematopoiesis leading to hematological disorders and malignancies.

The receptor tyrosine kinase FLT3 can undergo a series of mutations, including the activating internal tandem duplication (ITD) in the juxtamembrane region and point mutations in the tyrosine kinase domain such as at the activation loop residue D835. FLT3 is a target for acute myeloid leukemia (AML) therapy, as the FLT3-ITD mutation is present in approximately 24% of AML patients and it is associated with very poor prognosis. See C. Thiede et al., Blood 2002, 99, 4326; P. D. Kottaridis et al., Leukemia & lymphoma 2003, 44, 905. However, additional acquired mutations of FLT3, including D835 or “gatekeeper” F691 mutations that have been identified in clinical patients who showed resistance/relapse to FLT3 inhibitors sorafenib or quizartinib, can render most FLT3 inhibitors ineffective. See C. H. Man et al., Blood 2012, 119, 5133; C. C. Smith et al., Nature 2012, 485, 260. Additionally reported is that aberrant upregulation of other parallel pro-survival signaling pathways may render AML resistant to FLT3-targeted therapy. See W. Zhang et al., Clin.Cancer Res. 2014, 20, 2363.

Thus, there is a need for a treatment that would inhibit mutated FLT3 in hematologic malignancy patients who acquired the FLT 3 mutations.

Another tyrosine kinase, Bruton’s tyrosine kinase (BTK), is also found to be functionally important in regulating cell proliferation in blood cancers. BTK is found in B-cells and hematopoietic cells, rather than some T-cells, natural killer cells, plasma cells, etc. When BTK is stimulated by the B-cell membrane receptor (BCR) signals that are caused by various inflammatory responses or cancers, BTK plays an important role in production of cytokines such as TNF-αIL,-6, etc., as well as NF-KB by initiating downstream signaling such as phospholipase C gamma 2 (PLCγ).

In cancer treatments, it is known that BTK modifies BCR and B-cell surface proteins which generate anti-suicide signals. Thus, inhibition of BTK may bring about anticancer effects against cancers that are associated with BCR signaling such as lymphoma. The action mechanism of BTK inhibitor as an anti-inflammatory agent as well as an anti-cancer agent is thoroughly described in Nature Chemical Biology 2011, 7, 4.

These signaling pathways must be precisely regulated. Mutations in the gene encoding BTK cause an inherited B-cell specific immunodeficiency disease in humans, known as X-linked agammaglobulinemia (XLA) (Conley et al., Annu. Rev. Immunol. 27: 199-227, 2009). Aberrant BCR-mediated signaling may result in dysregulated B-cell activation leading to a number of autoimmune and inflammatory diseases. Preclinical studies show that BTK deficient mice are resistant to developing collagen- induced arthritis. Moreover, clinical studies of Rituxan, a CD20 antibody to deplete mature B-cells, reveal the key role of B-cells in a number of inflammatory diseases such as rheumatoid arthritis, systemic lupus erythematosus and multiple sclerosis (Gurcan et al., Int. Immunopharmacol. 9: 10-25, 2009). Therefore, BTK inhibitors can be used to treat autoimmune and/or inflammatory diseases.

In addition, aberrant activation or overexpression of BTK play important roles in pathogenesis of B-cell lymphomas, indicating that inhibition of BTK is useful in the treatment of hematological malignancies (Davis et al., Nature 463: 88-92, 2010). Preliminary clinical trial results showed that the BTK inhibitor ibrutinib (PCI-32765) was effective in treatment of several types of B-cell lymphoma (for example, 54^{th} American Society of Hematology (ASH) annual meeting abstract, December 2012: 686 The Bruton’s Tyrosine Kinase (BTK) Inhibitor, ibrutinib (PCI-32765), Has Preferential Activity in the ABC Subtype of Relapsed/Refractory De Novo Diffuse Large B-Cell Lymphoma (DLBCL): Interim Results of a Multicenter, Open-Label, Phase 1 Study). Because BTK plays a central role as a mediator in multiple signal transduction pathways, inhibitors of BTK are of great interest as anti-inflammatory and/or anti-cancer agents (Mohamed et al., Immunol. Rev. 228: 58-73, 2009; Pan, Drug News perspect 21: 357-362, 2008; Rokosz et al., Expert Opin. Ther. Targets 12: 883-903, 2008; Uckun et al., Anti-cancer Agents Med. Chem. 7: 624-632, 2007; Lou et al, J. Med. Chem. 55(10): 4539-4550, 2012).

Ibrutinib chemically interacts with the cysteine 481 residue in the active site of BTK and inactivates the BTK enzyme. However, mutation of the cysteine 481 reside to a serine residue (BTK-C481S) results in resistance to ibrutinib, and the BTK-C481S has been clinically observed. This specific point mutation effectively eliminates the target of ibrutinib, thereby disabling ibrutinib as an effective drug. Among CLL patients being treated with ibrutinib, 51% discontinue its use by the four-year mark due to various reasons. For example, 24% discontinue use of ibrutinib due to intolerance, adverse events, infection, or death. 27% of patients discontinue its use due to disease progression (e.g., Richter’s, BTK-C481S mutation, PLCγ2 mutation), and approximately ⅓ of patients discontinuing ibrutinib have the C481S mutation. Therefore, there is a need to identify a new therapeutic for treating patients refractory, intolerant or resistant (including patients with mutant forms of BTK), particularly one that acts through a different mechanism than ibrutinib.

Existing therapeutics are typically ineffective in the context of targets that display various resistance phenotypes. As a result, such cancers have a poor prognosis for survival. It is therefore important to develop novel pharmaceutical agents that demonstrate affinity for multiple kinase targets, specifically those capable of dual inhibition of BTK and FLT3.

### SUMMARY OF THE INVENTION

The present disclosure relates to Compound 7, pharmaceutically acceptable salts, esters, prodrugs, hydrates, solvates and isomers thereof.

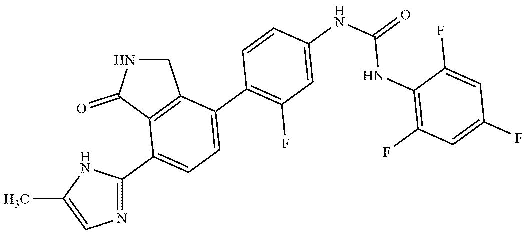 [SMILES: Cc1cnc(-c2ccc(-c3ccc(NC(=O)Nc4c(F)cc(F)cc4F)cc3F)c3c2C(=O)NC3)[nH]1]

In one embodiment, the present disclosure provides a method of inhibiting or reducing wild-type Fms-related kinase 3 (FLT3) activity or expression in a subject comprising administering Compound 7 or a pharmaceutically acceptable salt thereof to the subject. In one embodiment, the present disclosure provides a method of inhibiting or reducing mutated FLT3 activity or expression in a subject, comprises administering Compound 7 or a pharmaceutically acceptable salt thereof to the subject.

In one embodiment, the present disclosure provides a method of inhibiting or reducing wild type FLT3 activity or expression in human cells, comprises contacting Compound 7 or a pharmaceutically acceptable salt thereof with the human cells. In another embodiment, the present disclosure provides a method of inhibiting or reducing mutated FLT3 activity or expression in human cells, comprises contacting Compound 7 or a pharmaceutically acceptable salt thereof with the human cells.

In another embodiment, the present disclosure provides a method of inducing apoptosis of cells expressing wild type FLT3 in a subject in need thereof, comprises administering Compound 7 or a pharmaceutically acceptable salt thereof. In one embodiment, the present disclosure provides a method of inducing apoptosis of cells expressing mutated FLT3 in a subject in need thereof, comprises administering Compound 7 or a pharmaceutically acceptable salt thereof.

In one embodiment, the present disclosure provides a method of treating a hematologic malignancy associated with wild type FLT3 comprises administering Compound 7 or a pharmaceutically acceptable salt thereof to a subject in need thereof. In one embodiment, the method inhibits or reduces wild type FLT3 activity or expression. In another embodiment, the present disclosure provides a method of treating a hematologic malignancy associated with a mutated FLT3 comprises administering Compound 7 or a pharmaceutically acceptable salt thereof to a subject in need thereof. In one embodiment, the method inhibits or reduces mutant FLT3 activity or expression.

In one embodiment of any one of the methods disclosed herein, the mutated FLT3 comprises at least one point mutation. In one embodiment, the at least one point mutation is on one or more residues selected from the group consisting of D835, F691, K663, R834,, N841 and Y842. In another embodiment, the mutated FLT3 comprises at least one mutation at D835. In another embodiment, the mutated FLT3 comprises at least one mutation at F691. In one embodiment, the mutated FLT3 comprises at least one mutation at K663. In another embodiment, the mutated FLT3 comprises at least one mutation at N841. In another embodiment, the mutated FLT3 comprises at least one mutation at R834. In another embodiment, the mutated FLT3 comprises at least one mutation at Y842.

In one aspect of any one of the methods disclosed herein, the at least one point mutation is in the tyrosine kinase domain of FLT3. In another embodiment, the at least one point mutation is in the activation loop of FLT3. In one embodiment, the at least one point mutation is on one or more amino acid residue positions selected from the group consisting of 686, 687, 688, 689, 690, 691, 692, 693, 694, 695, and 696. In one embodiment, the mutated FLT3 has an additional ITD mutation. In one embodiment, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C.

In one embodiment of any one of the methods disclosed herein, the at least one point mutation is two or more point mutations present on the same allele. In one embodiment, the at least one point mutation is two or more point mutations present on different alleles.

In one embodiment of any one of the methods disclosed herein, the subject is a mammal. In another embodiment, the subject is a human.

In one embodiment of any methods disclosed herein for inhibiting or reducing wild type FLT3 or mutated FLT3 activity or expression in human cells, the human cells is human leukemia cell line. In one aspect, the human leukemia cell line is acute lymphocytic leukemia cell line, acute myeloid leukemia cell line, acute promyelocytic leukemia cell line, chronic lymphocytic leukemia cell line, chronic myeloid leukemia cell line, chronic neutrophilic leukemia cell line, acute undifferentiated leukemia cell line, anaplastic large-cell lymphoma cell line, prolymphocytic leukemia cell line, juvenile myelomonocytic leukemia cell line, adult T-cell acute lymphocytic leukemia cell line, acute myeloid leukemia cell line with trilineage myelodysplasia, mixed lineage leukemia cell line, eosinophilic leukemia cell line, or mantle cell lymphoma cell line. In one aspect, the human leukemia cell line is eosinophilic leukemia cell line. In one embodiment, the human leukemia cell line is acute myeloid leukemia cell line.

In one embodiment of any methods disclosed herein for treating a hematologic malignancy associated with wild type FLT3 or mutated FLT3 activity or expression, the hematologic malignancy is leukemia. In another embodiment, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, or mantle cell lymphoma. In another embodiment, the leukemia is eosinophilic leukemia. In another embodiment, the leukemia is acute myeloid leukemia.

In one embodiment, the present disclosure provides a method of treating a hematologic malignancy in a subject in need thereof, comprising administering a Compound 7 or a pharmaceutically acceptable salt thereof, wherein the subject shows resistance or relapse to an inhibitor of FLT3 activity or expression. In one embodiment, the inhibitor is quizartinib, gilteritinib, sunitinib, sorafenib, midostaurin, lestaurtinib, crenolanib, PLX3397, PLX3623, crenolanib, ponatinib, or pacritinib. In another emodiment, the inhibitor is quizartinib or gilteritinib.

In one embodiment, the hematologic malignancy is leukemia. In other embodiments, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, or mantle cell lymphoma. In a particular embodiment, the leukemia is eosinophilic leukemia. In another particular embodiment, the leukemia is acute myeloid leukemia.

In one embodiment, the present disclosure provides a method of inhibiting or reducing the abnormal (e.g., overexpressed) wild type or mutated BTK activity or expression in a subject in need thereof, comprising administering Compound 7 or a pharmaceutically acceptable salt thereof. In certain embodiments, the mutated BTK comprises at least one point mutation. For instance, the at least one point mutation may be on a cysteine residue (e.g., the cysteine residue is in the kinase domain of BTK). The subject may be a mammal, for example, a human. In certain embodiments, at least one point mutation is one or more residues selected from the group consisting of residues E41, P190, and C481. For example, the point mutation may be one or more selected from the group consisting of E41K, P190K, and C481S. In one embodiment, the point mutation at residue C481 is selected from C481S, C481R, C481T and/or C481Y.

In certain embodiments, the BTK mutant is resistant to inhibition by a covalent BTK inhibitor (e.g., ibrutinib and/or acalabrutinib or other covalent BTK inhibitors). In one embodiment, the activity of mutated BTK is inhibited less by a covalent irreversible BTK inhibitor than the activity of a wild type BTK by a covalent irreversible BTK inhibitor. For instance, the covalent irreversible BTK inhibitor has an IC50 at least 50% higher for the mutated BTK than for the wild type BTK. In certain embodiments, the BTK mutant is resistant to inhibition by a non-covalent BTK inhibitor. In certain embodiments, the BTK mutant is resistant to inhibition by a non-covalent BTK inhibitor.

In one embodiment, the point mutation on the cysteine is on only one allele of BTK. In another embodiment, the point mutation on the cysteine is on two alleles of BTK.

In one embodiment, the present disclosure provides a method for treating cancer in a subject in need thereof, comprising administering to a subject in need thereof Compound 7 or a pharmaceutically acceptable salt thereof, wherein the patient has a wild type (e.g., overexpressed wild-type) or mutant form of BTK.

In one embodiment, the present disclosure provides a method of treating a B cell malignancy in a subject in need thereof, comprising administering to the subject Compound 7 or a pharmaceutically acceptable salt thereof. In one embodiment, compound 7 inhibits the pathway activation of BTK, ERK, FLT3, AURK or AKT. In one embodiment, the subject has a mutant form of BTK. For example, the B cell malignancy is selected from one or more of the group consisting of mantle cell lymphoma (MCL), B-cell acute lymphoblastic leukemia (B-ALL), Burkitt’s lymphoma, chronic lymphocytic leukemia (CLL), and diffuse large B-cell lymphoma (DLBCL). In certain embodiments, Compound 7 inhibits and/or reduces the activity of any form (wild type or mutated) of an Aurora kinase. In one embodiment, the Aurora kinase is a mutated Aurora kinase. In one embodiment, the administration of Compound 7 induces cell death by mechanisms such as apoptosis. In another embodiment, the administration of Compound 7 induces polyploidies, autophagy, cell-cycle arrest or other non-apoptotic forms of cell death. In an embodiment, Compound 7 inhibits and/or reduces the activity or expression of wild type and/or mutant BTK. The mutated BTK comprises at least one point mutation, for example on a cysteine residue (e.g., residue C481).

In some embodiments, Compound 7 inhibits and/or reduces the activity of wild type Fms-related tyrosine kinase 3 (FLT3) activity or expression in a subject. In other embodiments, Compound 7 inhibits and/or reduces the activity of mutant Fms-related tyrosine kinase 3 (FLT3) activity or expression in the subject. The mutated FLT3 may comprise at least one point mutation. For example, the at least one point mutation is on one or more residues selected from the group consisting of D835, F691, K663, Y842 and N841. The mutated FLT3 may have an additional ITD mutation in one or two alleles.

In one embodiment, the present disclosure provides a method of inhibiting or reducing the abnormal (e.g., overexpressed) wild type or mutated BTK activity or expression in human cells, comprising contacting Compound 7 or a pharmaceutically acceptable salt thereof with the human cells. The mutated BTK may comprise at least one point mutation. In one embodiment, the at least one point mutation is on a cysteine residue. In one embodiment, the cysteine residue is in the kinase domain of BTK. The at least one point mutation is one or more selected from the group consisting of residues E41, P190, and C481. In one embodiment, the point mutation at residue cysteine 481 is selected from C481S, C481R, C481T and/or C481Y.

It should be appreciated that all combinations of the foregoing concepts and additional concepts discussed in greater detail below (provided such concepts are not mutually inconsistent) are contemplated as being part of the inventive subject matter disclosed herein. In particular, all combinations of claimed subject matter appearing at the end of this disclosure are contemplated as being part of the inventive subject matter disclosed herein. It should also be appreciated that terminology explicitly employed herein that also may appear in any disclosure incorporated by reference should be accorded a meaning most consistent with the particular concepts disclosed herein.

### BRIEF DESCRIPTION OF THE FIGURES

FIG. 1. is a Western-blot image showing that Compound 7 inhibits the FLT3 pathway in MV4-11 cells in a manner analogous to Quizartinib and in contrast to Ibrutinib.

FIG. 2. is a Western-blot image showing that Compound 7 inhibits the BTK pathway in MV4-11 cells at 0.5, 5.0, and 50 nM treatment amounts and the result is in accordance with the observed results from Ibrutinib treatment.

FIG. 3. is a Western-blot image showing that Compound 7 inhibits the BTK pathway in EOL-1 cells at 0.5, 5.0, and 50 nM treatment amounts and the result is in accordance with the observed results from Ibrutinib treatment.

FIG. 4A. to 4D. show a comparison of the cytotoxic effects of Compound 7, Ibrutinib and Quizartinib on FLT3-ITD (MV411 (FIG. 4A.) and MOLM-13 (FIG. 4B.)) and FLT3-WT (NOMO-1 (FIG. 4C.) and KG-1 (FIG. 4D.)) cells in the form of a dose-response curve as well as the corresponding IC₅₀ values.

FIG. 5. shows a rat pharmacokinetic data of mean plasma concentration of Compound 7 supplied at various doses by multiple routes of administration, either by IV, Oral suspension or Oral capsule.

FIG. 6. presents a mouse-xenograft model study of the efficacy of Compound 7 given at several different doses over a 22-day period compared to a control and Ibrutinib given at a single dosage over the same time period. FIG. 6. demonstrates that Compound 7 reduces tumor volume with increased dose when compared to the control or Ibrutinib treatment.

FIG. 7A shows early apoptotic cells in 0.5, 5.0, and 50 nM treatment of Compound 7, Ibrutinib, and Quizartinib.

FIG. 7B shows total apoptotic cells in 0.5, 5.0, and 50 nM treatment of Compound 7, Ibrutinib, and Quizartinib.

FIG. 7C shows live cells in 0.5, 5.0, and 50 nM treatment of Compound 7, Ibrutinib, and Quizartinib.

FIG. 8A is a Western-blot image showing that Compound 7 induces apoptosis in MV4-11 cells in comparison to Ibrutinib and Quizartinib treatments.

FIG. 8B is an Annexin V assay showing that Compound 7 induces apoptosis in MV4-11 cells in comparison to Ibrutinib and Quizartinib treatments.

FIG. 9 is a Western-blot image showing the reduction in the phosphorylated forms of various enzymes by Compound 7 in HEK293T transfected cells. Both wild-type and the C481S mutant form of BTK are inhibited by Compound 7 at concentrations of both 0.5 and 1.0 µM.

FIG. 10A and FIG. 10B show Western-blot images showing that Compound 7 inhibits BTK. 1 hour (FIG. 10A) and 24 hour (FIG. 10B) time points are shown.

FIGS. 11A - 11I show dose-response curves for Compound 7 and ibrutinib against the following cell lines: Mino (FIG. 11A), Granta-519 (FIG. 11B), Ramos (FIG. 11C), Daudi (FIG. 11D), SU-DHL6 (FIG. 11E), RL (FIG. 11F), Jeko-1 (FIG. 11G), RS411(FIG. 11H), and MHHCall4 (FIG. 11I). FIG. 11J is a dose response curve showing the cytotoxicity of Compound 7 against various indicated cell lines.

FIGS. 12A and 12B show that Compound 7 induces cellular apoptosis in Mino (FIG. 12A) and Ramos (FIG. 12B) B-cell malignant cell lines.

FIGS. 13A - 13C shows that Compound 7 is a highly potent Aurora kinase inhibitor. The cell lines from the top to bottom are: Mino (FIG. 13A), Ramos (FIG. 13B) and SU-DHL6 (FIG. 13C).

FIGS. 14A and 14B shows that Compound 7 induces polyploidy (left) and apoptosis (right) in Mino (FIG. 14A) and Ramos (FIG. 14B) B-cell malignant cell lines. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy.

FIG. 15 shows dose-response curves of the cytotoxicity of Compound 7 in various heme cell lines.

FIGS. 16A to 16E shows dose-response curves for Compound 7, Quizartinib, Gilteritinib, and Crenolanib against isogenic Ba/F3 cells transfected with the following FLT3 mutants: ITD (FIG. 16A), D835 (FIG. 16B), ITD + F691L (FIG. 16C), Ba/F3 FLT3 WT (FIG. 16D), and Ba/F3 ITD D835Y (FIG. 16E).

FIGS. 17A-J shows that Compound 7 time-dependently induces apoptosis in MV4-11 cells. FIG. 17A represents an Annexin V assay of MV4-11 cells only. FIG. 17B represents an Annexin V assay of MV4-11 cells treated with vehicle at 1 hour. FIG. 17C represents an Annexin V assay of MV4-11 cells treated with vehicle at 3 hour. FIG. 17D represents an Annexin V assay of MV4-11 cells treated with vehicle at 6 hours. FIG. 17E represents an Annexin V assay of MV4-11 cells treated with vehicle at 24 hour. FIG. 17F represents an Annexin V assay of MV4-11 cells treated with Compound 7 at 1 hour. FIG. 17G represents an Annexin V assay of MV4-11 cells treated with Compound 7 at 3 hours. FIG. 17H represents an Annexin V assay of MV4-11 cells treated with Compound 7 at 6 hours FIG. 17I represents an Annexin V assay of MV4-11 cells treated with Compound 7 at 24 hour. FIG. 17J represents a plot of the percentage of cells in late apoptotic, early apoptotic, or live state (CG = Compound 7).

FIGS. 18A-18D shows Compound 7 induces G0/G1 cell-cycle arrest in MV411 cells in a dose-dependent fashion. FIG. 18A is a graphical representation of the percentage of MV4-11 cells in either the G0/G1 state, S state, or G2/M state at varying concentrations. FIG. 18B shows flow cytograms with the y-axis representing 5-ethynyl-2′-deoxyuridine (EdU) fluorescence and the x-axis representing propidium iodide stained cells at the following concentrations of Compound 7: 0 nm (left) and 0.03 nm (right). FIG. 18C shows flow cytograms with the y-axis representing 5-ethynyl-2′-deoxyuridine (EdU) fluorescence and the x-axis representing propidium iodide stained cells at the following concentrations of Compound 7: 0.1 nm (left) and 0.3 nm (right). FIG. 18D shows a flow cytogram with the y-axis representing 5-ethynyl-2′-deoxyuridine (EdU) fluorescence and the x-axis representing propidium iodide stained cells at 0.5 nm of Compound 7.

FIGS. 19A - 19F show that Compound 7 induces G0/G1 cell-cycle arrest in MOLM-13 cells in a dose-dependent fashion. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy. FIG. 19A shows vehicle (top) and 0.1 nM (bottom) of Compound 7. FIG. 19B shows 0.3 nM (top) and 1 nM (bottom) of Compound 7. FIG. 19C shows vehicle (top) and 0.1 nM (bottom) of Ibrutinib. FIG. 19D shows 0.3 nM (top) and 1 nM (bottom) of Ibrutinib. FIG. 19E shows vehicle (top) and 0.1 nM (bottom) of Quizartinib. FIG. 19F shows 0.3 nM (top) and 1 nM (bottom) of Quizartinib.

FIG. 20A shows flow cytograms with the y-axis representing 5-ethynyl-2′-deoxyuridine (EdU) fluorescence and the x-axis representing propidium iodide stained cells with vehicle (top) and 0.03 nM of Compound 7 (bottom). FIG. 20B shows flow cytograms with the y-axis representing 5-ethynyl-2′-deoxyuridine (EdU) fluorescence and the x-axis representing propidium iodide stained cells with 0.1 nM (top) and 0.3 nM of Compound 7 (bottom). FIGS. 20C-20H show Compound 7 induces polyploidies in the following heme cell lines: NOMO-1 at 24 hours treated with vehicle (FIG. 20C top) and 3 nM (FIG. 20C bottom) of Compound 7, NOMO-1 at 24 hours treated with 30 nM (FIG. 20D top) and 300 nM (FIG. 20D bottom) of Compound 7, KG-1 at 24 hours treated with vehicle (FIG. 20E top) and 3 nM (FIG. 20E bottom) of Compound 7, KG-1 at 24 hours treated with 30 nM (FIG. 20F top) and 300 nM (FIG. 20F bottom) of Compound 7, KG-1 treated with vehicle (FIG. 20G top) and 3 nM (FIG. 20G bottom) of Compound 7, KG-1 at 24 hours treated with 30 nM (FIG. 20H top) and 300 nM (FIG. 20H bottom) of Compound 7. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy.

FIGS. 21A-21F show that Compound 7 was found to induce cell-cycle dysregulation in KG-1 cells in a dose-dependent fashion. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy. FIG. 21A shows vehicle (top) and 10 nM (bottom) of Compound 7. FIG. 21B shows 100 nM (top) and 1000 nM (bottom) of Compound 7. FIG. 21C shows vehicle (top) and 10 nM (bottom) of Ibrutinib. FIG. 21D shows 100 nM (top) and 1000 nM (bottom) of Ibrutinib. FIG. 21E shows vehicle (top) and 10 nM (bottom) of Quizartinib. FIG. 21F shows 100 nM (top) and 1000 nM (bottom) of Quizartinib.

FIGS. 22A - 22F show that Compound 7 was found to induce cell-cycle dysregulation in NOMO-1 cells in a dose-dependent fashion. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy. FIG. 22A shows vehicle (top) and 3 nM (bottom) of Compound 7. FIG. 22B shows 30 nM (top) and 300 nM (bottom) of Compound 7. FIG. 22C shows vehicle (top) and 3 nM (bottom) of Ibrutinib. FIG. 22D shows 30 nM (top) and 300 nM (bottom) of Ibrutinib. FIG. 22E shows vehicle (top) and 3 nM (bottom) of Quizartinib. FIG. 22F shows 30 nM (top) and 300 nM (bottom) of Quizartinib.

FIGS. 23A- 23Y shows that Compound 7 was found to induce cell-cycle dysregulation in and isogenic BA/F3 cells with various FLT3 mutations (indicated) in a dose-dependent fashion. FIG. 23A shows WT cells. FIG. 23B shows WT cells with vehicle. FIG. 23C shows WT cells with 3 nM of compound 7. FIG. 23D shows WT cells with 10 nM of compound 7. FIG. 23E shows WT cells with 30 nM of compound 7. FIG. 23F shows ITD + D835Y cells. FIG. 23G shows ITD + D835Y cells with vehicle. FIG. 23H shows ITD + D835Y cells with 3 nM of Compound 7. FIG. 23I shows ITD + D835Y cells with 10 nM of Compound 7. FIG. 23J shows ITD + D835Y cells with 30 nM of Compound 7. FIG. 23K shows ITD + F691L cells. FIG. 23L shows ITD + F691L cells with vehicle. FIG. 23M shows ITD + F691L cells with 3 nM of Compound 7. FIG. 23N shows ITD + F691L cells with 10 nM of Compound 7. FIG. 23O shows ITD + F691L cells with 30 nM of Compound 7. FIG. 23P shows D835Y cells. FIG. 23Q shows D835 cells with vehicle. FIG. 23R shows D835 cells with 3 nM of Compound 7. FIG. 23S shows D835 cells with 10 nM of Compound 7. FIG. 23T shows D835 cells with 30 nM of Compound 7. FIG. 23U shows ITD cells. FIG. 23V shows ITD cells with vehicle. FIG. 23W shows ITD cells with 0.1 nM of compound 7. FIG. 23X shows ITD cells with 0.3 nM of compound 7. FIG. 23Y shows ITD cells with 1 nM of compound 7.

FIG. 24 shows that relative to Aurora Kinase Inhibitor AT928, Compound 7 inhibits Aurora kinase activity and signaling in MV4-11 cells as indicated in the Western blot signature.

FIGS. 25A and 25B show that relative to ibrutinib and quizartinib, Compound 7 inhibits Aurora kinase activity (FIG. 25A) and signaling in FLT-3 WT cells (KG-1) (FIG. 25B) per the indicated Western blot signature.

FIG. 26A is a schematic showing that Compound 7 inhibits PDGFRA and FLT3 (WT) signaling in EOL-1 cells and FIG. 26B is a western blot showing that Compound 7 inhibits PDGFRA and FLT3 (WT) signaling in EOL-1 cells.

FIGS. 27A - 27D shows that Compound 7 interferes with cell cycle progression in RAMOS cells. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy. FIG. 27A shows the results of cells treated with vehicle. FIG. 27C shows the results of cells treated with 200 nM of Compound 7. FIG. 27D shows the results of cells treated with 1000 nM of Compound 7. The graph on the left of FIGS. 27A - 27D show cell count in the y-axis and propidium iodide stained cells in the x-axis and the graph on the right of FIGS. 27A - 27D show flow cytograms with the y-axis representing 5-ethynyl-2′ -deoxyuridine (EdU) fluorescence and the x-axis representing propidium iodide stained cells. FIG. 27B shows the results of cells treated with 20 nM of Compound 7.

FIGS. 28A - 28D show that Compound 7 interferes with cell cycle progression in Mino (FIG. 28A), RAMOS (FIG. 28B), GRANTA-519 (FIG. 28C), and SU-DHL-6 (FIG. 28D) cells. In FIG. 28A, from top to bottom, the concentration of Compound 7 against Mino cells was: vehicle, 0.1 nM, 1 nM, and 10 nM. In FIG. 28B, from top to bottom, the concentration of Compound 7 against RAMOS cells was: vehicle, 1 nM, 5 nM, and 10 nM. In FIG. 28C, from top to bottom, the concentration of Compound 7 against GRANTA-519 cells was: vehicle, 1 nM, 10 nM, and 100 nM. In FIG. 28D, from top to bottom, the concentration of Compound 7 against SU-DBL-6 cells was: vehicle, 1 nM, 10 nM, and 100 nM. The vertical line distinguishes the cells with normal DNA contents and polyploidy. To the left of the vertical line, the cells contain normal DNA content (<= 4N) and are in G0/G1, S, or G2/M phases of the cell cycle. To the right of the vertical line, the cells did not go through M phase and accumulated higher amount of DNA (> 4N), suggesting polyploidy.

FIG. 29A and FIG. 29B show that Compound 7 relative to Ibrutinib, inhibits BTK and Aurora kinase activity in Ramos cells.

FIGS. 30A and 30B show that relative to Ibrutinib, Compound 7 affects the BCR signaling in Ramos cells. 1 hour (FIG. 30A) and 6 hour (FIG. 30B) time points are shown.

FIGS. 31A and 31B shows that Compound 7 retains high activity at high serum concentration. Results for MV4-11 (FIG. 31A) and EOL1 (FIG. 31B) cells are shown.

### DETAILED DESCRIPTION OF THE INVENTION

The present disclosure, in one embodiment, provides a method of inhibiting or reducing wild-type or mutated Fms-related tyrosine kinase (FLT3) with a 2,3-dihydro-isoindole-1-one compound, or pharmaceutically acceptable salts, esters, prodrugs, hydrates, solvates and isomers thereof, for the treatment of cancer, such as blood cancers driven by aberrant activation of this kinase. Furthermore, in view of the foregoing challenges relating to treating B-cell malignancies associated with BTK, particularly mutated BTK (e.g., C481S BTK), Compound 7 was discovered to be surprisingly cytotoxic against B-cell malignant cell lines; many of which conventional therapeutic agents (e.g., ibrutinib) had little to no effect against. Unlike other conventional therapeutics (e.g., ibrutinib), Compound 7’s mechanism of action is believed to be through a non-covalent binding interaction with BTK, which is instrumental in preventing resistance to the BTK protein. Thus, the present disclosure , in one embodiment, provides a method of inhibiting or reducing the abnormal (e.g., overexpressed) wild type or mutated BTK activity or expression in a subject in need thereof, comprising administering Compound 7 or a pharmaceutically acceptable salt thereof. Further, Compound 7 inhibits additional kinases (AURK, c-Src and others) operative in B Cell malignancies that are not affected by ibrutinib.

### Definitions

It is to be understood that the terminology used herein is for the purpose of describing particular embodiments only and is not intended to be limiting.

Unless defined otherwise, all technical and scientific terms used herein have the same meaning as commonly understood to one of ordinary skill in the art to which the present application belongs. Although any methods and materials similar or equivalent to those described herein can be used in the practice or testing of the present application, representative methods and materials are herein described.

Reference throughout this specification to “one embodiment” or “an embodiment” means that a particular feature, structure or characteristic described in connection with the embodiment is included in at least one embodiment. Thus, the appearances of the phrases “in one embodiment” or “in an embodiment” in various places throughout this specification are not necessarily all referring to the same embodiment. Furthermore, the particular features, structures, or characteristics can be combined in any suitable manner in one or more embodiments. Also, as used in this specification and the appended claims, the singular forms “a,” “an,” and “the” include plural referents unless the content clearly dictates otherwise. It should also be noted that the term “or” is generally employed in its sense including “and/or” unless the content clearly dictates otherwise.

Unless otherwise indicated, all numbers expressing quantities of ingredients, reaction conditions, and so forth used in the specification and claims are to be understood as being modified in all instances by the term “about”. Accordingly, unless indicated to the contrary, the numerical parameters set forth in the present specification and attached claims are approximations that can vary depending upon the desired properties sought to be obtained by the present application.

Throughout the present specification, numerical ranges are provided for certain quantities. It is to be understood that these ranges comprise all subranges therein. Thus, the range “from 50 to 80” includes all possible ranges therein (e.g., 51-79, 52-78, 53-77, 54-76, 55-75, 60-70, etc.). Furthermore, all values within a given range can be an endpoint for the range encompassed thereby (e.g., the range 50-80 includes the ranges with endpoints such as 55-80, 50-75, etc.).

Compound 7 refers to 1-{3-fluoro-4-[7-(5-methyl-1H-imidazol-2-yl)-1-oxo-2,3-dihydro-1H-isoindol-4-yl]-phenyl}-3-(2,4,6-trifluorophenyl)urea and has the structure below:.

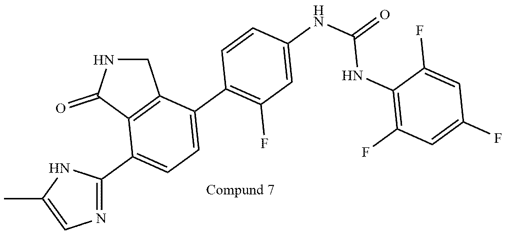

The present invention also includes pharmaceutically acceptable salts, esters, prodrugs, hydrates, solvates and isomers thereof, of compound 7.

A “pharmaceutically acceptable salt” includes both acid and base addition salts.

A pharmaceutically acceptable salt of Compound 7 may be a “pharmaceutically acceptable acid addition salt” derived from inorganic or organic acid, and such salt may be pharmaceutically acceptable nontoxic acid addition salt containing anion. For example, the salt may include acid addition salts formed by inorganic acids such as hydrochloric acid, sulfuric acid, nitric acid, phosphoric acid, hydrobromic acid, hydroiodic acid, and the like; organic carbonic acids such as tartaric acid, formic acid, citric acid, acetic acid, trichloroacetic acid, trifluoroacetic acid, gluconic acid, benzoic acid, lactic acid, fumaric acid, maleic acid, and the like; and sulfonic acids such as methanesulfonic acid, benzenesulfonic acid, p-toluenesulfonic acid, naphthalensulfonic acid, and the like.

The pharmaceutically acceptable salt of Compound 7 may be prepared by conventional methods well-known in the art. Specifically, the “pharmaceutically acceptable salt” in accordance of the present invention may be prepared by, e.g., dissolving the Compound 7 in a water-miscible organic solvent such as acetone, methanol, ethanol or acetonitrile and the like; adding an excessive amount of organic acid or an aqueous solution of inorganic acid thereto; precipitating or crystallizing the mixture thus obtained. Further, it may be prepared by further evaporating the solvent or excessive acid therefrom; and then drying the mixture or filtering the extract by using, e.g., a suction filter.

The term “ester” as used herein refers to a chemical moiety having chemical structure of —(R)ₙ—COOR′, wherein R and R′ are each independently selected from the group consisting of alkyl, cycloalkyl, aryl, heteroaryl ( connected to oxygen atom by aromatic ring) and heteroalicyclic (connected by aromatic ring), and n is 0 or 1, unless otherwise indicated.

The term “prodrug” as used herein refers to a precursor compound that will undergo metabolic activation in vivo to produce the parent drug. Prodrugs are often useful because they can be easily administered as compared to parent drugs thereof in some cases. For instance, some prodrugs are bioavailable via oral administration unlike parent drugs thereof often show poor bioavailability. Further, the prodrugs may show improved solubility in the pharmaceutical composition as compared to parent drugs thereof. For instance, Compound 7 may be administered in the form of an ester prodrug so as to increase drug delivery efficiency since the solubility of a drug can adversely affect the permeability across the cell membrane. Then, once the compound in the form of the ester prodrug enters a target cell, it may be metabolically hydrolyzed into a carboxylic acid and an active entity.

Hydrates or solvates of Compound 7 are included within the scope of the present invention. As used herein, “solvate” means a complex formed by solvation (the combination of solvent molecules with molecules or ions of the active agent of the present invention), or an aggregate that consists of a solute ion or molecule (the active agent of the present invention) with one or more solvent molecules. The solvent can be water, in which case the solvate can be a hydrate. Examples of hydrate include, but are not limited to, hemihydrate, monohydrate, dihydrate, trihydrate, hexahydrate, etc. It should be understood by one of ordinary skill in the art that the pharmaceutically acceptable salt of the present compound may also exist in a solvate form. The solvate is typically formed via hydration which is either part of the preparation of the present compound or through natural absorption of moisture by the anhydrous compound of the present invention. Solvates including hydrates may be consisting in stoichiometric ratios, for example, with two, three, four salt molecules per solvate or per hydrate molecule. Another possibility, for example, that two salt molecules are stoichiometric related to three, five, seven solvent or hydrate molecules. Solvents used for crystallization, such as alcohols, especially methanol and ethanol; aldehydes; ketones, especially acetone; esters, e.g. ethyl acetate; may be embedded in the crystal grating particularly pharmaceutically acceptable solvents.

The compounds of the disclosure or their pharmaceutically acceptable salts can contain one or more axes of chirality such that atropisomerization is possible. Atropisomers are stereoisomers arising because of hindered rotation about a single bond, where energy differences due to steric strain or other contributors create a barrier to rotation that is high enough to allow for isolation of individual conformers. The present disclosure is meant to include all such possible isomers, as well as their racemic and optically pure forms whether or not they are specifically depicted herein. Optically active isomers can be prepared using chiral synthons or chiral reagents, or resolved using conventional techniques, for example, chromatography and fractional crystallization. Conventional techniques for the preparation/isolation of individual atropisomers include chiral synthesis from a suitable optically pure precursor or resolution of the racemate (or the racemate of a salt or derivative) using, for example, chiral high pressure liquid chromatography (HPLC).

A “stereoisomer” refers to a compound made up of the same atoms bonded by the same bonds but having different three-dimensional structures, which are not interchangeable. The present invention contemplates various stereoisomers and mixtures thereof as it pertains to atropisomerism.

As used herein, aberrant activation of protein kinases is meant to include divergent, abnormal, atypical, anomalous or irregular kinase behavior that leads to a disease, disorder, or condition. Said diseases, disorders, and conditions, may include cancer, inflammation associated with rheumatoid arthritis and osteoarthritis, asthma, allergy, atopic dermatitis, or psoriasis, but not limited hereto. In the case of cancer, the disease, disorder, and condition can be characterized by uncontrolled cell proliferation.

Specific examples of cancer caused by aberrant activation of protein kinases includes, but are not limited to, ABL (Abelson tyrosine kinase), ACK (Activated cdc42-associated kinase), AXL, Aurora, BLK (B lymphoid tyrosine kinase), RMX (Bone marrow Xlinked kinase), BTK (Bruton’s tyrosine kinase), CDK (Cyclin-dependent kinase), CSK (C-Src kinase), DDR (Discoidin domain receptor), EPHA (Ephrin type A receptor kinase), FER (Fer(fps/fes related) tyrosine kinase), FES (Feline sarcoma oncogene), FGFR (Fibroblast growth factor receptor), FGR, FLT (Fms-like tyrosine kinase), FRK (Fyn-related kinase), FYN, HCK (Hemopoietic cell kinase), IRR (Insulin-receptor-related-receptor), ITK (Interleukin 2-inducible T cell kinase), JAK (Janus kinase), KDR (Kinase insert domain receptor), KIT, LCK (Lymphocyte-specific protein tyrosine kinase), LYN, MAPK (Mitogen activated protein kinase), MER (c-Mer proto-oncogene tyrosine kinase), MET, MINK (Misshapen-like kinase), MNK (MAPK-interacting kinase), MST (Mammalian sterile 20-like kinase), MUSK (Muscle-specific kinase), PDGFR (Platelet-derived growth factor receptor), PLK (Polo-like kinase), RET (Rearranged during transfection), RON, SRC (Steroid receptor coactivator), SRM (Spermidine synthase), TIE (Tyrosine kinase with immunoglobulin and EGF repeats), SYK (Spleen tyrosine kinase), TNK1 (Tyrosine kinase, non-receptor, 1), TRK (Tropomyosinreceptor-kinase), TNIK (TRAF2 and NCK interacting kinase) and the like.

The terms “treat”, “treating” or “treatment” in reference to a particular disease or disorder includes prevention of the disease or disorder, and/or lessening, improving, ameliorating or abrogating the symptoms and/or pathology of the disease or disorder. Generally, the terms as used herein refer to ameliorating, alleviating, lessening, and removing symptoms of a disease or condition. Compound 7 herein may be in a therapeutically effective amount in a formulation or medicament, which is an amount that can lead to a biological effect, such as apoptosis of certain cells (e.g., cancer cells), reduction of proliferation of certain cells, or lead to ameliorating, alleviating, lessening, or removing symptoms of a disease or condition, for example. The terms also can refer to reducing or stopping a cell proliferation rate (e.g., slowing or halting tumor growth) or reducing the number of proliferating cancer cells (e.g., removing part or all of a tumor).

When treatment as described above refers to prevention of a disease, disorder, or condition, said treatment is termed prophylactic. Administration of said prophylactic agent can occur prior to the manifestation of symptoms characteristic of a proliferative disorder, such that a disease or disorder is prevented or, alternatively, delayed in its progression.

As used herein, the terms “inhibiting” or “reducing” cell proliferation is meant to slow down, to decrease, or, for example, to stop the amount of cell proliferation, as measured using methods known to those of ordinary skill in the art, by, for example, 10%, 20%, 30%, 40%, 50%, 60%, 70%, 80%, 90%, 95%, or 100%, when compared to proliferating cells that are not subjected to the methods, compositions, and combinations of the present application.

As used herein, the term “apoptosis” refers to an intrinsic cell self-destruction or suicide program. In response to a triggering stimulus, cells undergo a cascade of events including cell shrinkage, blebbing of cell membranes and chromatic condensation and fragmentation. These events culminate in cell conversion to clusters of membrane-bound particles (apoptotic bodies), which are thereafter engulfed by macrophages.

As used herein, “polyploidy” or “polyploidy” refers to a condition in which a cell has a number of chromosomes that is some multiple of the monoploid number (“n”) greater than the usual diploid number (“2n”). The term “polyploid cells,” or “polyploidy cells” refers to cells in a polyploidy condition. In other words, the polyploid cell or organism has three or more times the monoploid chromosome number. In humans, the usual monoploid number of chromosomes is 23 and the usual diploid number of chromosomes is 46.

“Mammal” includes humans and both domestic animals such as laboratory animals and household pets (e.g., cats, dogs, swine, cattle, sheep, goats, horses, rabbits), and non-domestic animals such as wildlife and the like. The term “patient” or “subject” as used herein, includes humans and animals.

“Non-mammal” includes a non-mammalian invertebrate and non-mammalian vertebrate, such as a bird (e.g., a chicken or duck) or a fish.

A “pharmaceutical composition” refers to a formulation of a compound of the disclosure and a medium generally accepted in the art for the delivery of the biologically active compound to mammals, e.g., humans. Such a medium includes all pharmaceutically acceptable carriers, diluents or excipients therefor.

“An “effective amount” refers to a therapeutically effective amount or a prophylactically effective amount. A “therapeutically effective amount” refers to an amount effective, at dosages and for periods of time necessary, to achieve the desired therapeutic result, such as reduced tumor size, increased life span or increased life expectancy. A therapeutically effective amount of a compound can vary according to factors such as the disease state, age, sex, and weight of the subject, and the ability of the compound to elicit a desired response in the subject. Dosage regimens can be adjusted to provide the optimum therapeutic response. A therapeutically effective amount is also one in which any toxic or detrimental effects of the compound are outweighed by the therapeutically beneficial effects. A “prophylactically effective amount” refers to an amount effective, at dosages and for periods of time necessary, to achieve the desired prophylactic result, such as smaller tumors or slower cell proliferation. Typically, a prophylactic dose is used in subjects prior to or at an earlier stage of disease, so that a prophylactically effective amount can be less than a therapeutically effective amount.

The term “Bruton’s tyrosine kinase,” or BTK, as used herein, refers to Bruton’s tyrosine kinase from Homo sapiens, as disclosed in, e.g., U.S. Pat. No. 6,326,469 (GenBank Accession No. NP 000052).

The term “covalent BTK inhibitor”, as used herein, refers to an inhibitor that reacts with BTK to form a covalent complex. In some embodiments, the covalent BTK inhibitor is an irreversible BTK inhibitor.

The term “non-covalent BTK inhibitor”, as used herein, refers to an inhibitor that reacts with BTK to form a non-covalent complex or interaction. In some embodiments, the non-covalent BTK inhibitor is a reversible BTK inhibitor.

### Methods

In some embodiments, the present disclosure provides a method of inhibiting or reducing wild type or mutated Fms-related tyrosine kinase 3 (FLT3) activity or expression in a subject comprising administering Compound 7 or a pharmaceutically acceptable salt thereof to a subject in need thereof. In one embodiment, the method of inhibits or reduces FLT3 activity or expression in a subject in need thereof by administration of Compound 7.

Fms-related tyrosine kinase 3 (FLT3) refers to a protein encoded by the FLT3 gene. Wild-type FLT3 refers to the protein in a non-mutated form. FLT3 can undergo a series of mutations, including the activating internal tandem duplication (ITD) in the juxtamembrane region and point mutations in the tyrosine kinase domain or the activation loop of FLT3. Point mutations occur when a single base pair in a DNA sequence is modified. For instance, F691L is meant to define a change from phenyalanine to leucine for the amino acid at position 691.

In one embodiment, the mutated FLT3 has an additional ITD mutation. In one embodiment, ITD-mutation is associated with very poor prognosis in FTD-driven hematologic cancers, such as AML.

In another embodiment of any methods disclosed herein, mutated FLT3 comprises at least one point mutation. In another embodiment, the at least one point mutation is on one or more amino acid residue positions selected from the group consisting of 686, 687, 688, 689, 690, 691, 692, 693, 694, 695, and 696. In another embodiment, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-ITD-F691L, FLT3-K663Q, FLT3-N841I, FLT3-D835G, FLT3-Y842C, and FLT3-ITD-Y842C. In other embodiments, the at least one point mutation is two or more point mutations on the same allele or on different alleles.

In one embodiment of any methods disclosed herein, at least one point mutation is on amino acid residue position 686. In one embodiment, at least one point mutation is on amino acid residue position 687. In one embodiment, at least one point mutation is on amino acid residue position 688. In one embodiment, at least one point mutation is on amino acid residue position 689. In one embodiment, at least one point mutation is on amino acid residue position 690. In one embodiment, at least one point mutation is on amino acid residue position 691. In one embodiment, at least one point mutation is on amino acid residue position 692. In one embodiment, at least one point mutation is on amino acid residue position 693. In one embodiment, at least one point mutation is on amino acid residue position 694. In one embodiment, at least one point mutation is on amino acid residue position 695. In one embodiment, at least one point mutation is on amino acid residue position 696. In another embodiment, the at least one point mutations in on an amino residue that corresponds to position any residues 686-696.

In another embodiment, mutated FLT3 is FLT3-D835H. In another embodiment, mutated FLT3 is FLT3-D835V. In another embodiment, mutated FLT3 is FLT3-D835Y. In another embodiment, mutated FLT3 is FLT3-ITD-D835V. In another embodiment, mutated FLT3 is FLT3-ITD-D835Y. In another embodiment, mutated FLT3 is FLT3-ITD-D835H. In another embodiment, mutated FLT3 is FLT3-ITD-F691L. In another embodiment, mutated FLT3 is FLT3-K663Q. In another embodiment, mutated FLT3 is FLT3-N841I. In another embodiment, mutated FLT3 is FLT3-D835G, FLT3-Y842C, and/or FLT3-ITD-Y842C.

FLT3 is one of the targets for cancer therapy. Examples of diseases, disorders, and conditions related to aberrant activation of FLT3 include those resulting from over stimulation of FLT3 due to mutations in FLT3, or disorders resulting from abnormally high amount of FLT3 activity due to abnormally high amount of mutations in FLT3. Without bound to any theory, over-activity of FLT3 has been implicated in the pathogenesis of many diseases, including cancers. Cancers affiliated with over-activity of FLT3 include, but are not limited to, myeloproliferative disorders, such as thrombocytopenia, essential thrombocytosis (ET), agnogenic myeloid metaplasia, myelofibrosis (MF), myelofibrosis with myeloid metaplasia (MMM), chronic idiopathic myelofibrosis (UIMF), and polycythemia vera (PV), the cytopenias, and pre-malignant myelodysplastic syndromes; cancers such as glioma cancers, lung cancers, breast cancers, colorectal cancers, prostate cancers, gastric cancers, esophageal cancers, colon cancers, pancreatic cancers, ovarian cancers, and hematological malignancies, including myelodysplasia, multiple myeloma, leukemias, and lymphomas.

In one embodiment, the present disclosure provides a method of treating a hematologic malignancy associated with wild type FLT3 comprises administering Compound 7 or a pharmaceutically acceptable salt thereof to a subject in need thereof. In another embodiment, the present disclosure provides a method of treating a hematologic malignancy associated with a mutated FLT3 comprises administering Compound 7 or a pharmaceutically acceptable salt thereof to a subject in need thereof.

In one embodiment of any one of the methods disclosed herein, examples of hematological malignancies include, but are not limited to, leukemias, lymphomas, Hodgkin’s disease, and myeloma. Also, acute lymphocytic leukemia (ALL), acute myeloid leukemia (AML), acute promyelocytic leukemia (APL), chronic lymphocytic leukemia (CLL), chronic myeloid leukemia (CML), chronic neutrophilic leukemia (CNL), acute undifferentiated leukemia (AUL), anaplastic large-cell lymphoma (ALCL), prolymphocytic leukemia (PML), juvenile myelomonocytic leukemia (JMML), adult T-cell ALL, AML, with trilineage myelodysplasia (AMLITMDS), mixed lineage leukemia (MLL), myelodysplastic syndromes (MDSs), myeloproliferative disorders (MPD), and multiple myeloma (MM).

In one embodiment, the present disclosure provides a method of treating leukemia associated with a wild type or mutated FLT3 comprises administering Compound 7 or a pharmaceutically acceptable salt thereof to a subject in need thereof. In one embodiment, leukemia is AML.

In one embodiment, the present disclosure provides a method of inhibiting or reducing wild type or mutated FLT3 activity or expression in human cells with Compound 7 or a pharmaceutically acceptable salt thereof. In another embodiment, the present disclosure provides a method of inhibiting or reducing mutated FLT3 activity or expression in human cells by contacting Compound 7 with the human cells.

In one embodiment, the human cells in human leukemia cell line. In another embodiment, the human leukemia cell line is acute lymphocytic leukemia cell line, acute myeloid leukemia cell line, acute promyelocytic leukemia cell line, chronic lymphocytic leukemia cell line, chronic myeloid leukemia cell line, chronic neutrophilic leukemia cell line, acute undifferentiated leukemia cell line, anaplastic large-cell lymphoma cell line, prolymphocytic leukemia cell line, juvenile myelomonocytic leukemia cell line, adult T-cell acute lymphocytic leukemia cell line, acute myeloid leukemia cell line with trilineage myelodysplasia, mixed lineage leukemia cell line, eosinophilic leukemia cell line, or mantle cell lymphoma cell line.

In particular embodiments, the human leukemia cell line is eosinophilic leukemia. In another embodiment, the human leukemia cell line is and acute myeloid leukemia. Both of these blood cancers are known to be FLT3-driven. In one embodiment, MV4-11, MUTZ-11, MOLM-13, and PL-21 are acute myeloid leukemia cell lines harboring an FLT3-ITD mutation.

Treatment methods provide both prophylactic and therapeutic methods for treating a subject at risk or susceptible to developing a cell proliferative disorder driven by aberrant kinase activity of FLT3. In one example, the invention provides methods for preventing a cell proliferative disorder related to FLT3, comprising administration of a prophylactically effective amount of Compound 7 or a pharmaceutically acceptable salt thereof or a pharmaceutical composition comprising Compound 7 to a subject in need thereof. In one embodiment, prophylactic treatment can occur prior to the manifestation of symptoms characteristic of the FLT3 driven cell proliferative disorder, such that a disease or disorder is prevented or, alternatively, delayed in its progression.

In one embodiment, the present disclosure provides a method of treating a hematologic malignancy in a subject in need thereof, comprising administering a Compound 7 or a pharmaceutically acceptable salt thereof, wherein the subject shows resistance or relapse to an inhibitor of FLT3 activity or expression. In one embodiment, the inhibitor is quizartinib, gilteritinib, sunitinib, sorafenib, midostaurin, lestaurtinib, crenolanib, PLX3397, PLX3623, crenolanib, ponatinib, or pacritinib. In another emodiment, the inhibitor is quizartinib or gilteritinib.

In one embodiment, the hematologic malignancy is leukemia. In other embodiments, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, or mantle cell lymphoma. In a particular embodiment, the leukemia is eosinophilic leukemia. In another particular embodiment, the leukemia is acute myeloid leukemia.

In one embodiment, the method induces apoptosis of cells expressing wild type FLT3 in a subject in need thereof, comprising administering Compound 7 or a pharmaceutically acceptable salt thereof to the subject. In one embodiment, the present disclosure provides a method of inducing apoptosis of cells expressing mutated FLT3 in a subject in need thereof, comprises administering Compound 7 or a pharmaceutically acceptable salt thereof.

In another embodiment, the methods include methods for treating cancer in a subject in need thereof, comprising administering to a subject in need thereof Compound 7 or a pharmaceutically acceptable salt thereof, wherein the subject has a mutant form of FLT3. In another embodiment, the mutated FLT3 comprises at least one point mutation. In another embodiment, the at least one point mutation is on one or more residues selected from the group consisting of D835, F691, K663, Y842 and N841. In another embodiment, the mutated FLT3 comprises at least one mutation at D835. In another embodiment, the mutated FLT3 comprises at least one mutation at F691. In another embodiment, the mutated FLT3 comprises at least one mutation at K663. In another embodiment, the mutated FLT3 comprises at least one mutation at N841. In another embodiment, the at least one point mutation is in the tyrosine kinase domain of FLT3. In another embodiment, the at least one point mutation is in the activation loop of FLT3. In another embodiment, the at least one point mutation is on one or more amino acid residue positions selected from the group consisting of 686, 687, 688, 689, 690, 691, 692, 693, 694, 695, and 696. In another embodiment, the mutated FLT3 is an ITD mutation. In another embodiment, the mutated FLT3 comprises at least one point mutation and an ITD mutation. In another embodiment, the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-K663Q, FLT3-ITD-K663Q FLT3-N841I, FLT3-ITD-N841I, FLT-3R834Q FLT3-ITD-834Q, FLT3-D835G, FLT3-ITD-D835G, FLT3-Y842C, and FLT3-ITD-Y842C. In another embodiment, the at least one point mutation is two or more point mutations present on the same allele. In another embodiment, the at least one point mutation is two or more point mutations present on different alleles. In another embodiment, the subject is a mammal. In another embodiment the subject is human. In another embodiment, the cancer is leukemia. In another embodiment, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma. In another specific embodiment, the leukemia is acute myeloid leukemia.

In one embodiment, the present invention provides a method of dually inhibiting or reducing activity or expression of kinases in a subject in need thereof by administering Compound 7 or a pharmaceutically acceptable salt thereof to the subject. In another embodiment, a method of dually inhibiting or reducing activity or expression is for mutated Fms-related tyrosine kinase 3 (FLT3) activity and inhibiting or reducing activity or expression of Bruton’s Tyrosine Kinase (BTK) activity in combination, in a subject comprising administering Compound 7 or a pharmaceutically acceptable salt thereof. In one embodiment, Compound 7 inhibits or reduces both FLT3 (including wild type and mutated FLT3) and BTK activity. Targeting multi-kinase pathways is a method that can improve outcomes in cancers with poor prognosis.

In some embodiments, the present disclosure provides a method of inhibiting or reducing the abnormal (e.g., overexpressed) wild-type or mutated BTK activity or expression in a subject in need thereof, comprising administering Compound 7 or a pharmaceutically acceptable salt thereof to the subject.

In certain embodiments, the BTK is wild-type. In one embodiment, the wild-type BTK is abnormal (e.g., overexpressed) in a subject. In another embodiment, the wild-type BTK is overactive or hyperactive in a subject.

In certain embodiments, the BTK is mutated BTK. The BTK mutation may be caused by a variety of factors, which are readily apparent to a skilled artisan, such as an insertion mutation, deletion mutation, and substitution mutation (e.g., point mutation). In one embodiment, the mutated BTK comprises at least one point mutation.

A variety of point mutations are contemplated within the scope of the present disclosure. For instance, the at least one point mutation may be to any residue on the BTK. In some embodiments, a mutation within the BTK gene includes a mutation at amino acid positions L11, K12, S14, K19, F25, K27, R28, R33, Y39, Y40, E41, I61, V64, R82, Q103, V113, S115, T117, Q127, C154, C155, T184, P189, P190, Y223, W251, R288, L295, G302, R307, D308, V319, Y334, L358, Y361, H362, H364, N365, S366, L369, I370M, R372, L408, G414, Y418, I429, K430, E445, G462, Y476, M477, C481, C502, C506, A508, M509, L512, L518, R520, D521, A523, R525, N526, V535, L542, R544, Y551, F559, R562, W563, E567, 5578, W581, A582, F583, M587, E589, S592, G594, Y598, A607, G613, Y617, P619, A622, V626, M630, C633, R641, F644, L647, L652, V1065, and/or A1185. In some embodiments, a mutation within the BTK gene is selected from among L11P, K12R, S14F, K19E, F25S, K27R, R28H, R28C, R28P, T33P, Y3S9, Y40C, Y40N, E41K, I61N, V64F, V64D, R82K, Q103Q5FSSVR, V113D, S115F, T117P, Q127H, C1545, C155G, T184P, P189A, Y223F, W251L, R288W, R288Q, L295P, G302E, R307K, R307G, R307T, D308E, V319A, Y334S, L358F, Y361C, H362Q, H364P, N365Y, S366F, L369F, I370M, R372G, L408P, G414R, Y418H, I429N, K430E, E445D, G462D, G462V, Y476D, M477R, C481S, C502F, C502W, C506Y, C506R, A508D, M5091, M509V, L512P, L512Q, L518R, R520Q, D521G, D521H, D521N, A523E, R525G, R525P, R525Q, N526K, V535F, L542P, R544G, R544K, Y551F, F559S, R562W, R562P, W563L, E567K, S578Y, W581R, A582V, F583S, M587L, E589D, E589K, E589G, S592P, G594E, Y598C, A607D, G613D, Y617E, P619A, P619S, A622P, V626G, M630I, M630K, M630T, C633Y, R641C, F644L, F644S, L647P, L652P, V10651, and A1185V. In one embodiment, the at least one point mutation is on a cysteine residue. In one embodiment, the cysteine residue is in the kinase domain of BTK. In some embodiments, the at least one point mutation is one or more selected from the group consisting of residues E41, P190, and C481. In some embodiments, the mutation in BTK is at amino acid position 481 (i.e., C481). The C481 point mutation may be substituted with any amino acid moiety. In some embodiments, the mutation in BTK is C481S. In one embodiment, the point mutation at residue C481 is selected from C481S, C481R, C481T and/or C481Y. In one embodiment, the at least one point mutation is one or more selected from the group consisting of E41K, P190K, and C481S.

In some embodiments, the B cell lymphoma is characterized by a plurality of cells having a mutant BTK polypeptide. In some embodiments, the mutant BTK polypeptides contain one or more amino acid substitutions that confers resistance to inhibition by a covalent and/or irreversible BTK inhibitor. In some embodiments, the mutant BTK polypeptides contain one or more amino acid substitutions that confers resistance to inhibition by a covalent and/or irreversible BTK inhibitor that covalently binds to cysteine at amino acid position 481 of a wild-type BTK. In some embodiments, the mutant BTK polypeptides contain one or more amino acid substitutions that confers resistance to inhibition by a covalent and/or irreversible BTK inhibitor selected from PCI-32765 (ibrutinib), PCI-45292, PCI-45466, AVL-101/CC-101 (Avila Therapeutics/Celgene Corporation), AVL-263/CC-263 (Avila Therapeutics/Celgene Corporation), AVL-292/CC-292 (Avila Therapeutics/Celgene Corporation), AVL-291/CC-291 (Avila Therapeutics/Celgene Corporation), CNX 774 (Avila Therapeutics), BMS-488516 (Bristol-Myers Squibb), BMS-509744 (Bristol-Myers Squibb), CGI-1746 (CGI Pharma/Gilead Sciences), CGI-560 (CGI Pharma/Gilead Sciences), CTA-056, GDC-0834 (Genentech), HY-11066 (also, CTK4I7891, HMS3265G21, HMS3265G22, HMS3265H21, HMS3265H22, 439574-61-5, AG-F-54930), ONO-4059 (Ono Pharmaceutical Co., Ltd.), ONO-WG37 (Ono Pharmaceutical Co., Ltd.), PLS-123 (Peking University), RN486 (Hoffmann-La Roche), HM71224 (Hanmi Pharmaceutical Company Limited), LFM-A13, BGB-3111 (Beigene), KBP-7536 (KBP BioSciences), ACP-196 (Acerta Pharma) or JTE-051 (Japan Tobacco Inc). In some embodiments, the mutant BTK polypeptides contain one or more amino acid substitutions that confers resistance to inhibition by ibrutinib. In some instances, the plurality of cells comprises at least two cells. In certain embodiments, the BTK mutant contain one or more amino acid substitutions that confers resistance to inhibition by a non-covalent BTK inhibitor. In certain embodiments, the BTK mutant contain one or more amino acid substitutions that confers resistance to inhibition by a reversible BTK inhibitor.

As described above in some embodiments, the modification comprises a substitution or a deletion of the amino acid at amino acid position 481 compared to a wild type BTK. In some embodiments, the modification comprises substitution of the amino acid at position 481 compared to a wild type BTK. In some embodiments, the modification is a substitution of cysteine to an amino acid selected from among leucine, isoleucine, valine, alanine, glycine, methionine, serine, threonine, phenylalanine, tryptophan, lysine, arginine, histidine, proline, tyrosine, asparagine, glutamine, aspartic acid and glutamic acid at amino acid position 481 of the BTK polypeptide. In some embodiments, the modification is a substitution of cysteine to an amino acid selected from among serine, methionine, or threonine at amino acid position 481 of the BTK polypeptide. In some embodiments, the modification is a substitution of cysteine to serine at amino acid position 481 of the BTK polypeptide (“C481S”).

In some embodiments, the mutations in BTK confer resistance in a B cell proliferative disorder to a TEC inhibitor (e.g. ITK inhibitor, BTK inhibitor such as ibrutinib). In some embodiments, C481S mutation in BTK confers resistance in a B cell proliferative disorder to a TEC inhibitor (e.g. ITK inhibitor, BTK inhibitor such as ibrutinib). In some embodiments, the mutations in BTK confer resistance in a B cell proliferative disorder to a covalent BTK inhibitor. In some embodiments, the mutations in BTK confer resistance in a B cell proliferative disorder to ibrutinib and acalabrutinib.

In one embodiment, the activity of mutated BTK is inhibited less by a covalent irreversible BTK inhibitor than the activity of a wild type BTK by a covalent irreversible BTK inhibitor. The covalent irreversible BTK inhibitor may have an IC₅₀ from at least about 1% higher to at least about 1000% higher for the mutated BTK than for the wild type BTK. For example, the covalent irreversible BTK inhibitor may have an IC₅₀ from at least about 1%, 2%, 3%, 4%, 5%, 6%, 7%, 8%, 9%, 10%, 11%, 12%, 13%, 14%, 15%, 16%, 17%, 18%, 19%, 20%, 21%, 22%, 23%, 24%, 25%, 26%, 27%, 28%, 29%, 30%, 31%, 32%, 33%, 34%, 35%, 36%, 37%, 38%, 39%, 40%, 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89%, 90%, 91%, 92%, 93%, 94%, 95%, 96%, 97%, 98%, 99%, 100%, 110%, 120%, 130%, 140%, 150%, 160%, 170%, 180%, 190%, 200%, 210%, 220%, 230%, 240%, 250%, 260%, 270%, 280%, 290%, 300%, 310%, 320%, 330%, 340%, 350%, 360%, 370%, 380%, 390%, 400%, 410%, 420%, 430%, 440%, 450%, 460%, 470%, 480%, 490%, 500%, 510%, 520%, 530%, 540%, 550%, 560%, 570%, 580%, 590%, 600%, 610%, 620%, 630%, 640%, 650%, 660%, 670%, 680%, 690%, 700%, 710%, 720%, 730%, 740%, 750%, 760%, 770%, 780%, 790%, 800%, 810%, 820%, 830%, 840%, 850%, 860%, 870%, 880%, 890%, 900%, 910%, 920%, 930%, 940%, 950%, 960%, 970%, 980%, 990%, to at least about 1000% higher for the mutated BTK than for the wild type BTK. In one embodiment, the covalent irreversible BTK inhibitor has an IC₅₀ at least 50% higher for the mutated BTK than for the wild type BTK. In one embodiment, the irreversible covalent BTK inhibitor is ibrutinib and/or acalabrutinib. For example, the irreversible covalent BTK inhibitor is ibrutinib.

In one embodiment, the point mutation is on only one allele of BTK. In another embodiment, the point mutation is on two alleles of BTK. In one embodiment, the point mutation on the cysteine is on only one allele of BTK. In another embodiment, the point mutation on the cysteine is on two alleles of BTK. In one embodiment, the point mutation on C481 is on only one allele of BTK. In another embodiment, the point mutation on C481 is on two alleles of BTK. In one embodiment, the C481S point mutation is on only one allele of BTK. In another embodiment, the C481S point mutation is on two alleles of BTK.

In one embodiment, the subject is a mammal. In one embodiment, the subject is a human.

Another aspect of the present disclosure is directed to a method for treating cancer in a subject in need thereof, comprising administering to a subject in need thereof Compound 7 or a pharmaceutically acceptable salt thereof, wherein the subject has a mutant form of BTK.

Another aspect of the present disclosure is directed to a method of treating a B cell malignancy in a subject in need thereof, comprising administering to the subject Compound 7 or a pharmaceutically acceptable salt thereof. In one embodiment, the subject has a mutant form of BTK.

In some embodiments, the B cell malignancy is a chronic lymphocytic leukemia (CLL), small lymphocytic lymphoma (SLL), high risk CLL, or a non-CLL/SLL lymphoma. In some embodiments, the B cell proliferative disorder is follicular lymphoma, diffuse large B-cell lymphoma (DLBCL), mantle cell lymphoma, Waldenstrom’s macroglobulinemia, multiple myeloma, marginal zone lymphoma, Burkitt’s lymphoma, non-Burkitt high grade B cell lymphoma, or extranodal marginal zone B cell lymphoma. In some embodiments, the B cell malignancy is acute or chronic myelogenous (or myeloid) leukemia, myelodysplastic syndrome, or acute lymphoblastic leukemia. In some embodiments, the B cell malignancy is relapsed or refractory diffuse large B-cell lymphoma (DLBCL), relapsed or refractory mantle cell lymphoma, relapsed or refractory follicular lymphoma, relapsed or refractory CLL; relapsed or refractory SLL; relapsed or refractory multiple myeloma. In some embodiments, the B cell malignancy is a B cell proliferative disorder that is classified as high-risk. In some embodiments, the B cell malignancy is high risk CLL or high risk SLL.

Accordingly, in one embodiment, the treated B cell malignancy is selected from one or more of the group consisting of mantle cell lymphoma (MCL), B-cell acute lymphoblastic leukemia (B-ALL), Burkitt’s lymphoma, chronic lymphocytic leukemia (CLL), and diffuse large B-cell lymphoma (DLBCL). In one embodiment, the treated B cell malignancy is mantle cell lymphoma (MCL). In another embodiment, the treated B cell malignancy is B-cell acute lymphoblastic leukemia (B-ALL). In one embodiment, the treated B cell malignancy is Burkitt’s lymphoma. In one embodiment, the treated B cell malignancy is chronic lymphocytic leukemia (CLL). In one embodiment, the treated B cell malignancy is mantle cell lymphoma (MCL). In one embodiment, the treated B cell malignancy is diffuse large B-cell lymphoma (DLBCL).

B-cell malignancies are neoplasms of the blood and encompass, inter alia, non-Hodgkin lymphoma, multiple myeloma, and leukemia. They can originate either in the lymphatic tissues (as in the case of lymphoma) or in the bone marrow (as in the case of leukemia and myeloma), and they all are involved with the uncontrolled growth of lymphocytes or white blood cells. There are many subtypes ofB cell proliferative disorders. The disease course and treatment ofB cell proliferative disorder is dependent on the B cell proliferative disorder subtype; however, even within each subtype the clinical presentation, morphologic appearance, and response to therapy is heterogeneous.

In another embodiment, the methods may also include treating a hematologic malignancy by administering Compound 7, or a pharmaceutically salt thereof, to a patient in need thereof. In another embodiment, the hematologic malignancy is leukemia. In another embodiment, the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma. In a specific embodiment, the leukemia is acute myeloid leukemia.

Malignant lymphomas are neoplastic transformations of cells that reside predominantly within lymphoid tissues. Two groups of malignant lymphomas are Hodgkin’s lymphoma and non-Hodgkin’s lymphoma (NHL). Both types of lymphomas infiltrate reticuloendothelial tissues. However, they differ in the neoplastic cell of origin, site of disease, presence of systemic symptoms, and response to treatment.

In one embodiment, Compound 7 inhibits and/or reduces the activity of Aurora kinase. Aurora kinases (Aurora-A, Aurora-B, Aurora-C) are serine/threonine protein kinases that are essential for proliferating cells and have been identified as key regulators of different steps in mitosis and meiosis, ranging from the formation of the mitotic spindle to cytokinesis. Aurora family kinases are critical for cell division, and have been closely linked to tumorigenesis and cancer susceptibility. In various human cancers over-expression and/or upregulation of kinase activity of Aurora-A, Aurora-B and/or Aurora C has been observed. Over-expression of Aurora kinases correlates clinically with cancer progression and poor survival prognosis. Aurora kinases are involved in phosphorylation events (e.g. phosphorylation of histone H3) that regulate the cell cycle. Misregulation of the cell cycle can lead to cellular proliferation and other abnormalities.

Without being bound by any particular theory, inhibition of BTK and/or Aurora kinase may lead to failure in cytokinesis and abnormal exit from mitosis, which could result in polyploidy cells, cell cycle arrest, and ultimately apoptosis.

Accordingly, in one embodiment, the administration of Compound 7 induces polyploidies. In another embodiment, the administration of Compound 7 induces apoptosis. For example, in one embodiment, a cell is contacted with an effective amount of Compound 7, thereby causing cellular polyploidies and/or cell cycle arrest and/or apoptosis. The cells may be cancer or tumor cells. Accordingly, in one embodiment, the administration of Compound 7 induces apoptosis in cancer and/or tumor cells. In yet another embodiment, the administration of Compound 7 induces apoptosis in cancer and/or tumor cells expressing mutant BTK (e.g., C481S).

In any of the embodiments of the present disclosure, Compound 7 may inhibit and/or reduce the activity or expression of wild type BTK and/or mutant BTK. Accordingly, in some embodiments, Compound 7 inhibits and/or reduces the activity or expression of wild type BTK. In other embodiments, Compound 7 inhibits and/or reduces the activity or expression of mutant BTK. The mutant BTK may comprise at least one point mutation. In one embodiment, the mutant BTK comprises at least one point mutation on a cysteine residue. In one embodiment, the mutant BTK comprises at least one point mutation at residue C481. In one embodiment, the mutant BTK comprises at least a C481S mutation.

Fms-related tyrosine kinase 3 (FLT3) refers to a protein encoded by the FLT3 gene. Wild-type FLT3 refers to the protein in a non-mutated form. FLT3 can undergo a series of mutations, including the activating internal tandem duplication (ITD) in the juxtamembrane region and point mutations in the tyrosine kinase domain or the activation loop of FLT3. In any of the embodiments of the present disclosure, Compound 7 inhibits and/or reduces the activity of wild type and/or mutant Fms-related tyrosine kinase 3 (FLT3) activity or expression in a subject. In one embodiment, Compound 7 inhibits and/or reduces the activity of wild type Fms-related tyrosine kinase 3 (FLT3) activity or expression in a subject. In another embodiment, Compound 7 inhibits and/or reduces the activity of mutant Fms-related tyrosine kinase 3 (FLT3) activity or expression in a subject. The mutant FLT3 may comprise at least one point mutation. In one embodiment, the mutated FLT3 comprises at least one point mutation on one or more residues selected from the group consisting of D835, F691, K663, Y842 and N841. The mutated FLT3 may be FLT3-ITD. The mutated FLT3 may further comprise an additional ITD mutation.

Another aspect of the present disclosure is directed to a method of inhibiting or reducing the abnormal (e.g., overexpressed) wild-type or mutated BTK activity or expression in human cells, comprising contacting Compound 7 or a pharmaceutically acceptable salt thereof with the human cells.

In one embodiment, the mutated BTK comprises at least one point mutation. A variety of point mutations are contemplated within the scope of the present disclosure and are described above. For instance, the at least one point mutation may be to any residue on the BTK. In one embodiment, the at least one point mutation is on a cysteine residue. In one embodiment, the cysteine residue is in the kinase domain of BTK. In some embodiments, the at least one point mutation is one or more selected from the group consisting of residues E41, P190, and C481. In some embodiments, the mutation in BTK is at amino acid position 481. The C481 point mutation may be substituted with any amino acid moiety. In some embodiments, the mutation in BTK is C481S. In one embodiment, the point mutation at residue C481 is selected from C481S, C481R, C481T and/or C481Y. In one embodiment, the at least one point mutation is one or more selected from the group consisting of E41K, P190K, and C481S.

### Formulations

The effective amount of Compound 7, pharmaceutically acceptable salts, esters, prodrugs, hydrates, solvates and isomers thereof, or a pharmaceutical composition comprising Compound 7 or a pharmaceutically acceptable salt thereof may be determined by one skilled in the art based on known methods.

In one embodiment, a pharmaceutical composition or a pharmaceutical formulation of the present disclosure comprises Compound 7 or a pharmaceutically acceptable salt thereof, and a pharmaceutically acceptable carrier. Pharmaceutically acceptable carrier, diluent or excipient includes without limitation any adjuvant, carrier, excipient, glidant, sweetening agent, diluent, preservative, dye/colorant, flavor enhancer, surfactant, wetting agent, dispersing agent, suspending agent, stabilizer, isotonic agent, solvent, or emulsifier which has been approved by the United States Food and Drug Administration as being acceptable for use in humans or domestic animals.

In one embodiment, suitable pharmaceutically acceptable carriers include, but are not limited to, inert solid fillers or diluents and sterile aqueous or organic solutions. Pharmaceutically acceptable carriers are well known to those skilled in the art and include, but are not limited to, from about 0.01 to about 0.1 M and preferably 0.05 M phosphate buffer or 0.8% saline. Such pharmaceutically acceptable carriers can be aqueous or non-aqueous solutions, suspensions and emulsions. Examples of non-aqueous solvents suitable for use in the present application include, but are not limited to, propylene glycol, polyethylene glycol, vegetable oils such as olive oil, and injectable organic esters such as ethyl oleate.

Aqueous carriers suitable for use in the present application include, but are not limited to, water, ethanol, alcoholic/aqueous solutions, glycerol, emulsions or suspensions, including saline and buffered media. Oral carriers can be elixirs, syrups, capsules, tablets and the like.

Liquid carriers suitable for use in the present application can be used in preparing solutions, suspensions, emulsions, syrups, elixirs and pressurized compounds. The active ingredient can be dissolved or suspended in a pharmaceutically acceptable liquid carrier such as water, an organic solvent, a mixture of both or pharmaceutically acceptable oils or fats. The liquid carrier can contain other suitable pharmaceutical additives such as solubilizers, emulsifiers, buffers, preservatives, sweeteners, flavoring agents, suspending agents, thickening agents, colors, viscosity regulators, stabilizers or osmo-regulators.

Liquid carriers suitable for use in the present application include, but are not limited to, water (partially containing additives as above, e.g. cellulose derivatives, preferably sodium carboxymethyl cellulose solution), alcohols (including monohydric alcohols and polyhydric alcohols, e.g. glycols) and their derivatives, and oils (e.g. fractionated coconut oil and arachis oil). For parenteral administration, the carrier can also include an oily ester such as ethyl oleate and isopropyl myristate. Sterile liquid carriers are useful in sterile liquid form comprising compounds for parenteral administration. The liquid carrier for pressurized compounds disclosed herein can be halogenated hydrocarbon or other pharmaceutically acceptable propellent.

Solid carriers suitable for use in the present application include, but are not limited to, inert substances such as lactose, starch, glucose, methyl-cellulose, magnesium stearate, dicalcium phosphate, mannitol and the like. A solid carrier can further include one or more substances acting as flavoring agents, lubricants, solubilizers, suspending agents, fillers, glidants, compression aids, binders or tablet-disintegrating agents; it can also be an encapsulating material. In powders, the carrier can be a finely divided solid which is in admixture with the finely divided active compound. In tablets, the active compound is mixed with a carrier having the necessary compression properties in suitable proportions and compacted in the shape and size desired. The powders and tablets preferably contain up to 99% of the active compound. Suitable solid carriers include, for example, calcium phosphate, magnesium stearate, talc, sugars, lactose, dextrin, starch, gelatin, cellulose, polyvinylpyrrolidine, low melting waxes and ion exchange resins. A tablet may be made by compression or molding, optionally with one or more accessory ingredients. Compressed tablets may be prepared by compressing in a suitable machine the active ingredient in a free flowing form such as a powder or granules, optionally mixed with a binder (e.g., povidone, gelatin, hydroxypropylmethyl cellulose), lubricant, inert diluent, preservative, disintegrant (e.g., sodium starch glycolate, cross-linked povidone, cross-linked sodium carboxymethyl cellulose) surface active or dispersing agent. Molded tablets may be made by molding in a suitable machine a mixture of the powdered compound moistened with an inert liquid diluent. The tablets may optionally be coated or scored and may be formulated so as to provide slow or controlled release of the active ingredient therein using, for example, hydroxypropyl methylcellulose in varying proportions to provide the desired release profile. Tablets may optionally be provided with an enteric coating, to provide release in parts of the gut other than the stomach.

Parenteral carriers suitable for use in the present application include, but are not limited to, sodium chloride solution, Ringer’s dextrose, dextrose and sodium chloride, lactated Ringer’s and fixed oils. Intravenous carriers include fluid and nutrient replenishers, electrolyte replenishers such as those based on Ringer’s dextrose and the like. Preservatives and other additives can also be present, such as, for example, antimicrobials, antioxidants, chelating agents, inert gases and the like.

Carriers suitable for use in the present application can be mixed as needed with disintegrants, diluents, granulating agents, lubricants, binders and the like using conventional techniques known in the art. The carriers can also be sterilized using methods that do not deleteriously react with the compounds, as is generally known in the art.

Diluents may be added to the formulations of the present invention. Diluents increase the bulk of a solid pharmaceutical composition and/or combination, and may make a pharmaceutical dosage form containing the composition and/or combination easier for the patient and care giver to handle. Diluents for solid compositions and/or combinations include, for example, microcrystalline cellulose (e.g., AVICEL), microfine cellulose, lactose, starch, pregelatinized starch, calcium carbonate, calcium sulfate, sugar, dextrates, dextrin, dextrose, dibasic calcium phosphate dihydrate, tribasic calcium phosphate, kaolin, magnesium carbonate, magnesium oxide, maltodextrin, mannitol, polymethacrylates (e.g., EUDRAGIT(r)), potassium chloride, powdered cellulose, sodium chloride, sorbitol, and talc.

For the purposes of this disclosure, the pharmaceutical composition of the present disclosure can be formulated for administration by a variety of means including orally, parenterally, by inhalation spray, topically, or rectally in formulations containing pharmaceutically acceptable carriers, adjuvants and vehicles. The term parenteral as used here includes subcutaneous, intravenous, intramuscular, and intraarterial injections with a variety of infusion techniques. Intraarterial and intravenous injection as used herein includes administration through catheters.

The pharmaceutical composition of the present invention may be prepared into any type of formulation and drug delivery system by using any of the conventional methods well-known in the art. The inventive pharmaceutical composition may be formulated into injectable formulations, which may be administered by routes including intrathecal, intraventricular, intravenous, intraperitoneal, intranasal, intraocular, intramuscular, subcutaneous or intraosseous. Also, it may also be administered orally, or parenterally through the rectum, the intestines or the mucous membrane in the nasal cavity (see Gennaro, A. R., ed. (1995) Remington’s Pharmaceutical Sciences). Preferably, the composition is administered topically, instead of enterally. For instance, the composition may be injected, or delivered via a targeted drug delivery system such as a reservoir formulation or a sustained release formulation.

The pharmaceutical formulation of the present invention may be prepared by any well-known methods in the art, such as mixing, dissolving, granulating, dragee-making, levigating, emulsifying, encapsulating, entrapping, or lyophilizing processes. As mentioned above, the compositions of the present invention may include one or more physiologically acceptable carriers such as excipients and adjuvants that facilitate processing of active molecules into preparations for pharmaceutical use.

Proper formulation is dependent upon the route of administration chosen. For injection, for example, the composition may be formulated in an aqueous solution, preferably in physiologically compatible buffers such as Hank’s solution, Ringer’s solution, or physiological saline buffer. For transmucosal or nasal administration, penetrants appropriate to the barrier to be permeated are used in the formulation. Such penetrants are generally known in the art. In a one embodiment of the present invention, the inventive compound may be prepared in an oral formulation. For oral administration, the compounds can be formulated readily by combining the active compounds with pharmaceutically acceptable carriers known in the art. Such carriers enable the disclosed compound to be formulated as tablets, pills, dragees, capsules, liquids, gels, syrups, slurries, suspensions and the like, for oral ingestion by a subject. The compounds may also be formulated in rectal compositions such as suppositories or retention enemas, e.g., containing conventional suppository bases such as cocoa butter or other glycerides.

Pharmaceutical preparations for oral use may be obtained as solid excipients, optionally grinding a resulting mixture, and processing the mixture of granules, after adding suitable adjuvants, if desired, to obtain tablets or dragee cores. Suitable excipients may be, in particular, fillers such as sugars, including lactose, sucrose, mannitol, or sorbitol; cellulose formulation such as maize starch, wheat starch, rice starch, potato starch, gelatin, gum tragacanth, methyl cellulose, hydroxypropylmethyl-cellulose, sodium carboxymethylcellulose, and/or polyvinylpyrrolidone (PVP) formulation. Also, disintegrating agents may be employed, such as cross-linked polyvinylpyrrolidone, agar, or alginic acid or a salt thereof such as sodium alginate. Also, wetting agents, such as sodium dodecyl sulfate and the like, may be added.

Dragee cores are provided with suitable coatings. For this purpose, concentrated sugar solutions may be used, which may optionally contain gum arabic, talc, polyvinylpyrrolidone, carbopol gel, polyethylene glycol, and/or titanium dioxide, lacquer solutions, and suitable organic solvents or solvent mixtures. Dyestuffs or pigments may be added to the tablets or dragee coatings for identification or to characterize different combinations of active compounds doses.

Pharmaceutical formulations for oral administration may include push-fit capsules made of gelatin, as well as soft, sealed capsules made of gelatin and a plasticizer, such as glycerol or sorbitol. The push-fit capsules can contain the active ingredients in admixture with filler such as lactose, binders such as starches, and/or lubricants such as talc or magnesium stearate and, optionally, stabilizers. In soft capsules, the active compounds may be dissolved or suspended in suitable liquids, such as fatty oils, liquid paraffin, or liquid polyethylene glycols. In addition, stabilizers may be added. All formulations for oral administration should be in dosages suitable for such administration.

In one embodiment, the compounds of the present invention may be administered transdermally, such as through a skin patch, or topically. In one aspect, the transdermal or topical formulations of the present invention can additionally comprise one or multiple penetration enhancers or other effectors, including agents that enhance migration of the delivered compound. Preferably, transdermal or topical administration may be used, e.g., in situations in which location specific delivery is desired.

For administration by inhalation, the compounds of the present invention may be conveniently delivered in the form of an aerosol spray presentation from pressurized packs or a nebulizer, with the use of a suitable propellant, e.g., dichlorodifluoromethane, trichlorofluoromethane, dichlorotetrafluoroethane, carbon dioxide, or any other suitable gas. In the case of a pressurized aerosol, the appropriate dosage unit may be determined by providing a valve to deliver a metered amount. Capsules and cartridges of, e.g., gelatin, for use in an inhaler or insufflators may be formulated. These typically contain a powder mix of the compound and a suitable powder base such as lactose or starch. Compositions formulated for parenteral administration by injection, e.g., by bolus injection or continuous infusion, can be presented in unit dosage form e.g., in ampoules or in multi-dose containers, with an added preservative. The compositions may take such forms as suspensions, solutions or emulsions in oily or aqueous vehicles, and may contain formulatory agents such as suspending, stabilizing and/or dispersing agents. Formulations for parenteral administration include aqueous solutions or other compositions in water-soluble form.

Suspensions of the active compounds may also be prepared as appropriate oily injection suspensions. Suitable lipophilic solvents or vehicles may include fatty oils such as sesame oil and synthetic fatty acid esters, such as ethyl oleate or triglycerides, or liposomes. Aqueous injection suspensions may contain substances that increase the viscosity of the suspension, such as sodium carboxymethyl cellulose, sorbitol, or dextran. Optionally, the suspension may also contain suitable stabilizers or agents that increase the solubility of the compounds to allow for the preparation of highly concentrated solutions. Alternatively, the active ingredient may be in powder form for constitution with a suitable vehicle, e.g., sterile pyrogen-free water, before use.

As mentioned above, the compositions of the present invention may also be formulated as a reservoir formulation. Such long acting formulations may be administered by implantation (e.g., subcutaneous or intramuscular) or by intramuscular injection. Thus, for example, the inventive compounds may be formulated with suitable polymeric or hydrophobic materials (e.g., an emulsion in an acceptable oil) or ion exchange resins, or as sparingly soluble derivatives, e.g., a sparingly soluble salt.

For any composition used in the present methods of treatment, a therapeutically effective dose can be estimated initially using a variety of techniques well-known in the art. For example, based on information obtained from a cell culture assay, a dose can be formulated in animal models to achieve a circulating concentration range that includes the IC₅₀. Similarly, dosage ranges appropriate for human subjects can be determined, for example, using data obtained from cell culture assays and other animal studies.

A therapeutically effective dose of an agent refers to the amount of the agent that results in amelioration of symptoms or a prolongation of survival in a subject. Toxicity and therapeutic efficacy of such molecules can be determined by standard pharmaceutical procedures in cell cultures or experimental animals, for example, by determining the LD₅₀ (the dose lethal to 50% of the population) and the ED₅₀ (the dose therapeutically effective in 50% of the population). The dose ratio between toxic and therapeutic effects is the therapeutic index, which can be expressed as the ratio LD_{50O}/ED₅₀. Agents that exhibit high therapeutic indices are sought.

Dosages preferably fall within a range of circulating concentrations that includes the ED₅₀ with little or no toxicity. Dosages may vary within this range depending upon the dosage form employed and the route of administration utilized. The exact formulation, route of administration, and dosage should be chosen, according to methods well-known in the art, in view of the specifics of a subject’s condition.

In addition, the amount of agent or composition administered will be dependent on a variety of factors, including the age, weight, sex, health condition, degree of disease of the subject being treated, the severity of the affliction, the manner of administration, and the judgment of the prescribing physician.

The compound or pharmaceutical compositions of the present disclosure may be manufactured and/or administered in single or multiple unit dose forms.

Having now generally described the invention, the same will be more readily understood through reference to the following examples, which are provided by way of illustration and are not intended to be limiting of the present invention. Unless expressly stated otherwise, conditions and procedures performed as generally known in the art.

### EXAMPLES

### Synthesis: Material and Methods

Various starting materials may be prepared in accordance with conventional synthetic methods well-known in the art. Some of the starting materials are commercially available from manufacturers and suppliers of reagents, such as Aldrich, Sigma, TCI, Wako, Kanto,

Fluorchem, Acros, Abocado, Alfa, Fluka, etc., but not limited thereto.

The compounds of the present disclosure can be prepared from readily available starting materials by conventional methods and processes below. Different methods may also be used for manufacturing the inventive compounds, unless otherwise specified as typical or optimal process conditions (i.e., reaction temperature, time, molar ratio of reactants, solvents, pressures, etc.). The optimal reaction conditions may vary depending on the particular reactants or solvents employed. Such conditions, however, can be determined by the skilled in the art by conventional optimization process.

In addition, those of ordinary skill in the art recognize that some functional groups can be protected/deprotected using various protecting groups before a certain reaction takes place. Suitable conditions for protecting and/or deprotecting specific functional group, and the use of protecting groups are well-known in the art.

For example, various kinds of protecting groups are described in T.W. Greene and G.M. Wuts, Protecting Groups in Organic Synthesis, Second edition, Wiley, New York, 1991, and other references cited above.

In one embodiment of the present invention, Compound 7 of the present invention may be prepared by synthesizing an intermediate, Compound D, according to the Scheme 1 as shown below, and then subjecting Compound D through the procedure of Reaction Scheme 2. However, the method for synthesizing Compound D above is not limited to Reaction Scheme 1.

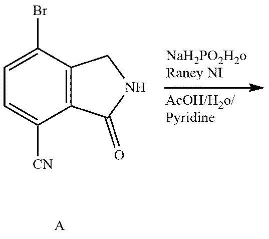

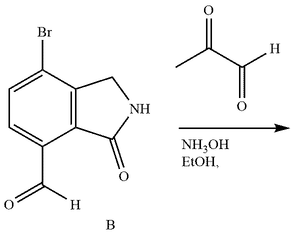

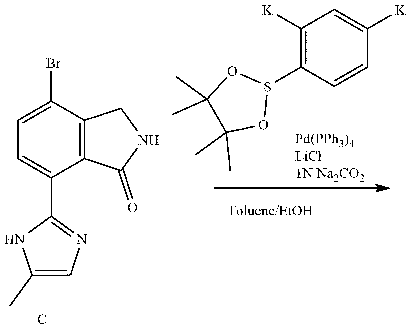

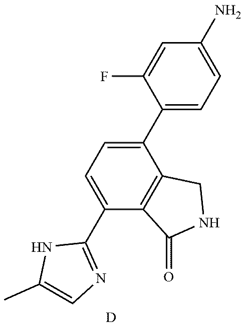

The method for preparing the starting material of Reaction Scheme 1, i.e., Compound A, is described in International Patent Publication W02012/014017, and the preparation of Compound D is described in U.S. Pat. Application Publication US2015/0336934

Example 1: Synthesis of 1-{3-fluoro-4-[7-(5-methyl-1H-imidazol-2-yl)-1-oxo-2,3-dihydro-1H-isoindol-4-yl]-phenyl}-3-(2,4,6-trifluoro-phenyl)-urea (Compound 7)

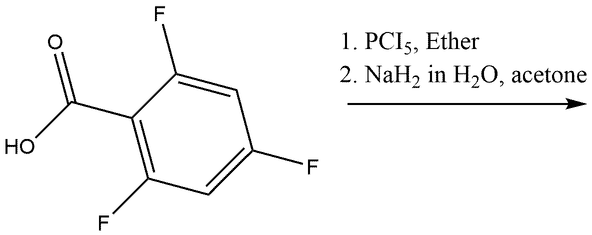

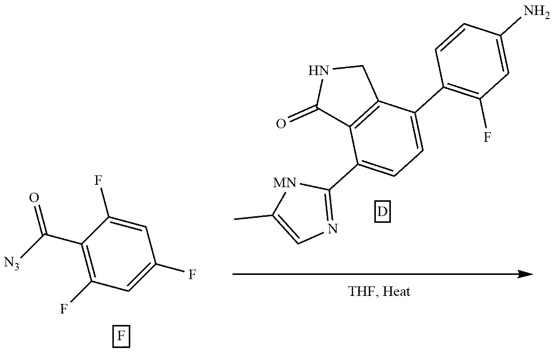

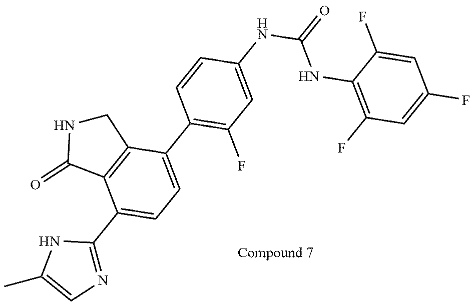

2,4,6-trifluorobenzoic acid (0.08 g, 0.45 mmol) was dispersed in diethyl ether (5.7 mL), slowly added with phosphorus pentachloride (PCl₅, 0.11 g, 0.52 mmol), and then stirred for 1 hour. Upon completion of the reaction, the organic solvent was concentrated under reduced pressure below room temperature, and then the reaction solution was diluted by adding acetone (3.8 mL). Subsequently, sodium azide (NaN₃, 0.035 g, 0.545 mmol) dissolved in water (0.28 mL) was slowly added to the reaction solution dropwise at 0° C. After stirring for 2 hours at room temperature, 2,4,6-trifluorobenzoyl azide thus formed was diluted with ethyl acetate, and then washed with water. The organic layer was dried over anhydrous magnesium sulfate, dispersed in THF (2 mL), added with THF (7.5 mL) containing 4-(4-amino-2-fluorophenyl)-7-(5-methyl-1H-imidazol-2-yl)isoindolin-1-one (Compound D, 0.073 g, 0.23 mmol), and then stirred for 3 hours at 90° C. Upon completion of the reaction, the solvent was concentrated under reduced pressure, and then purified by silica gel column chromatography (eluent:methylene chloride: methanol = 20:1) to obtain Compound 7 (0.026 g, yield: 23%). ¹H-NMR (300 MHz, DMSO-d): 14.46-14.37 (m 1H), 9.47-9.45 (br m, 1H), 9.37 (s, 1H), 8.45 (d, J=1.8 Hz, 1H), 8.30-8.27 (br m, 1H), 7.63-7.46 (m, 3H), 7.31-7.26 (m, 3H), 7.09-6.84 (m, 1H), 4.42 (s, 2H), 2.31-2.21 (m, 3H). LCMS [M+1]: 496.3.

### Example 2: Binding Constant of Compound 7 for Wild Type and Mutated FLT3 Kinase

Measurements of said kinase activity are referred to as binding constants or K_{d} values. The protocol for obtaining these values is hereby described. For most assays, kinase-tagged T7 phage strains were prepared in an E. coli host derived from the BL21 strain. E. coli were grown to log-phase and infected with T7 phage and incubated with shaking at 32° C. until lysis. The lysates were centrifuged and filtered to remove cell debris. The remaining kinases were produced in HEK-293 cells and subsequently tagged with DNA for qPCR detection. Streptavidin-coated magnetic beads were treated with biotinylated small molecule ligands for 30 minutes at room temperature to generate affinity resins for kinase assays. The liganded beads were blocked with excess biotin and washed with blocking buffer (SeaBlock (Pierce), 1% BSA, 0.05% Tween 20, 1 mM DTT) to remove unbound ligand and to reduce nonspecific binding. Binding reactions were assembled by combining kinases, liganded affinity beads, and test compounds in 1x binding buffer (20% SeaBlock, 0.17x PBS, 0.05% Tween 20, 6 mM DTT). Test compounds were prepared as 111X stocks in 100% DMSO. Kds were determined using an 11-point 3-fold compound dilution series with three DMSO control points. All compounds for K_{d} measurements are distributed by acoustic transfer (non-contact dispensing) in 100% DMSO. The compounds were then diluted directly into the assays such that the final concentration of DMSO was 0.9%. All reactions performed in polypropylene 384-well plate. Each was a final volume of 0.02 ml. The assay plates were incubated at room temperature with shaking for 1 hour and the affinity beads were washed with wash buffer (1x PBS, 0.05% Tween 20). The beads were then re-suspended in elution buffer (1x PBS, 0.05% Tween 20, 0.5 µM non-biotinylated affinity ligand), and incubated at room temperature with shaking for 30 minutes. The kinase concentration in the eluates was measured by qPCR.

Binding constant measured for Compound 7 and Quizartinib are shown in Table 1 below.

Table 1

Binding Constant (K_{d}) of Compound 7 and Quizartinib

| | Compound 7 | Quizartinib |
|---|---|---|
| FLT3(D835H) | 2.2 | 3.7 |
| FLT3(D835V) | 7.9 | N/A |
| FLT3(D835Y) | 4.2 | 7.1 |
| FLT3(ITD) | 3.1 | 8.8 |
| FLT3(ITD-D835V) | 1700 | N/A |
| FLT3(ITD-F691L) | 15 | N/A |
| FLT3(K663Q) | 0.55 | 2.2 |
| FLT3(N841I) | 0.76 | 4.1 |
| FLT3(R834Q) | 6.4 | N/A |
| FLT3(WT) | 0.24 | 1.3 |

### Example 3: Western Blot Analysis for Compound 7 in MV4-11 Cells

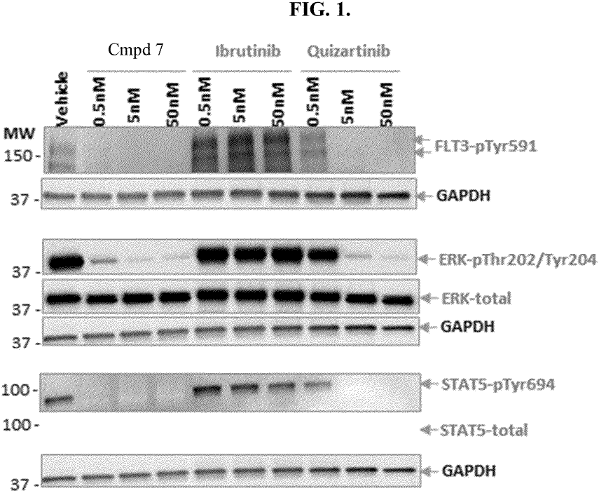

FIG. 1. shows the Western-blot analysis results. Without being bound to any theory, this demonstrates that Compound 7 inhibits FLT3 pathway in MV4-11 cells.

Example 4: Inhibition of FLT3 mutants with Compound 7

Table 2

IC₅₀ of Compound 7 in FLT3

| | IC₅₀ (nM) |
|---|---|
| FLT3(D835Y) | 166.5 |
| FLT3(ITD) | 0.8172 |
| FLT3(WT) | 8.746 |

Example 5: Cytotoxicity of Compound 7 on Heme cell lines

Table 3

Cytotoxicity of Compound 7

| Disease type | Cell lines | ICSO (µM) |
|---|---|---|
| | | Mean |
| AML | EOL-1 | 0.00004495 |
| AML | MV4-11 | 0.000238 |
| AML | Molm-13 | 0.0003951 |
| ALL | RS411 | 0.001508 |
| MCL | Mino | 0.005976 |
| MCL | GRANTA-519 | 0.01359 |
| Burkitt’s | Ramos | 0.01841 |
| AML | NOM0-1 | 0.02052 |
| AML | KG-1 | 0.02869 |
| MCL | Jeko- 1 | 0.07377 |
| DLBCL | SU-DHL6 | 0.1216 |
| Burkitt’s | Daudi | 0.1660 |
| AML | HL60 | 0.2796 |
| DLBCL | RL | 0.4262 |
| AML | SKM-1 | 0.8161 |
| AML | MUTZ-8 | 0.793700 |
| ALL | Jurkat | 1.59 |
| Burkitt’s | Raji | 2.03 |
| AML | THP-1 | 2.97 |
| CLL | MEC-1 | 3.80 |
| AML | HEL92.1.7 | 4.69 |
| CML | K562 | 12.94 |

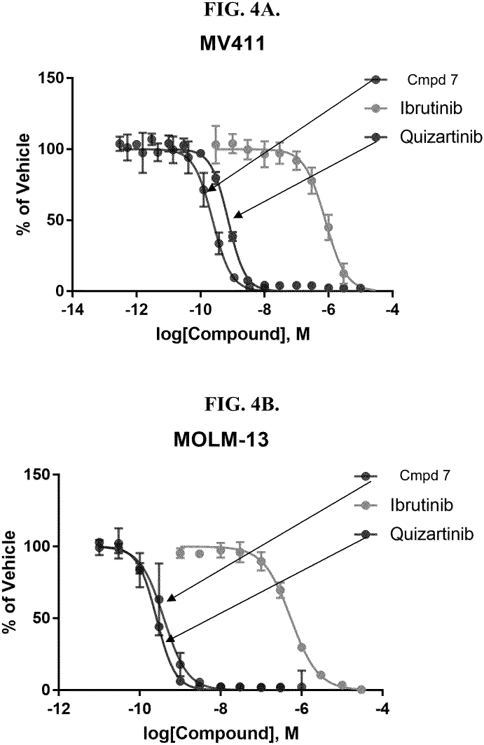

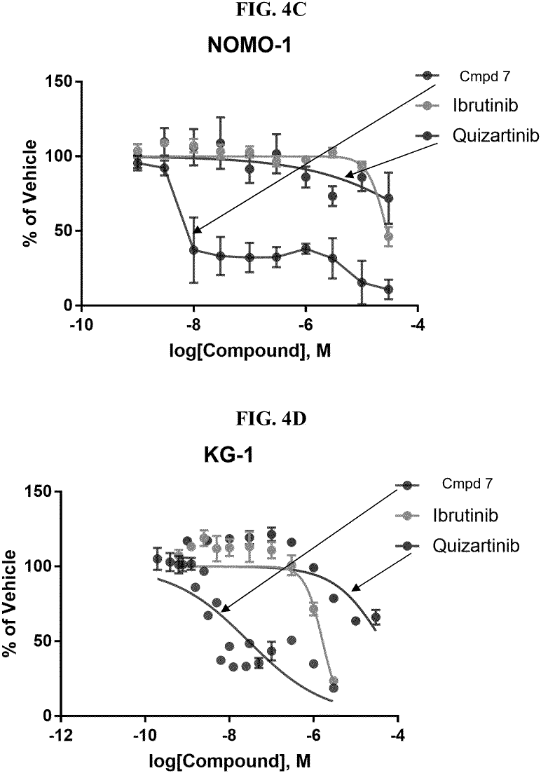

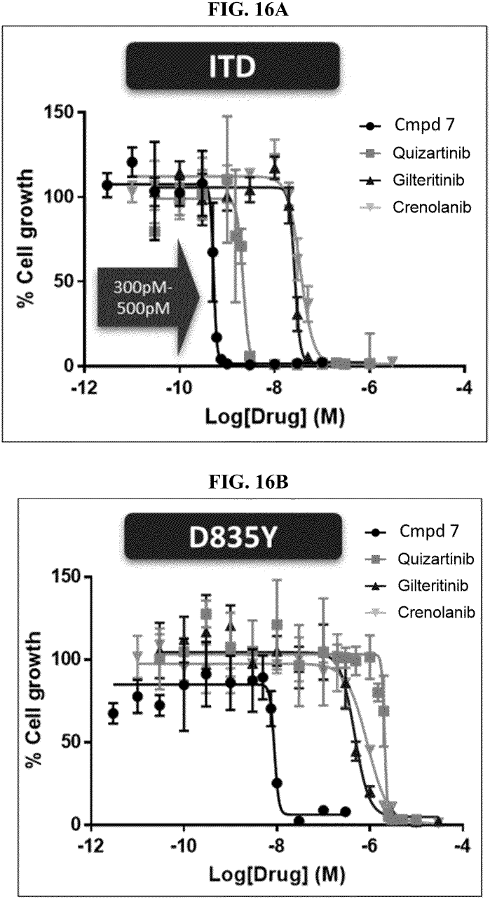

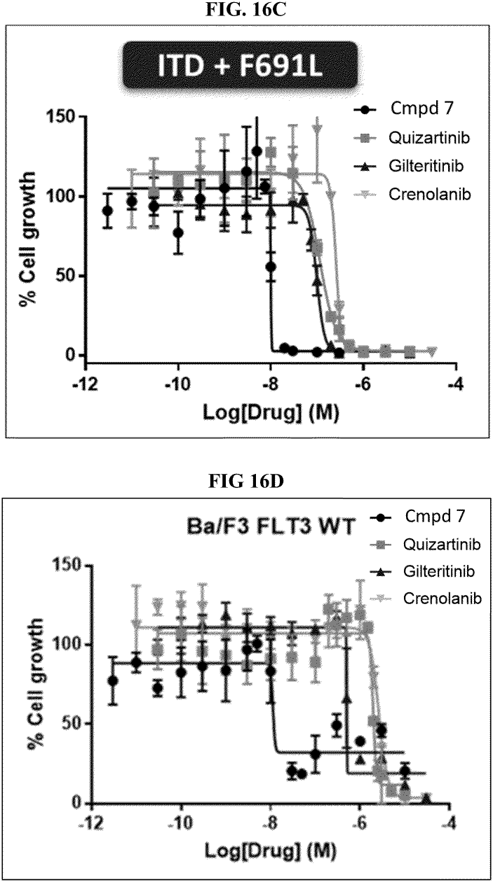

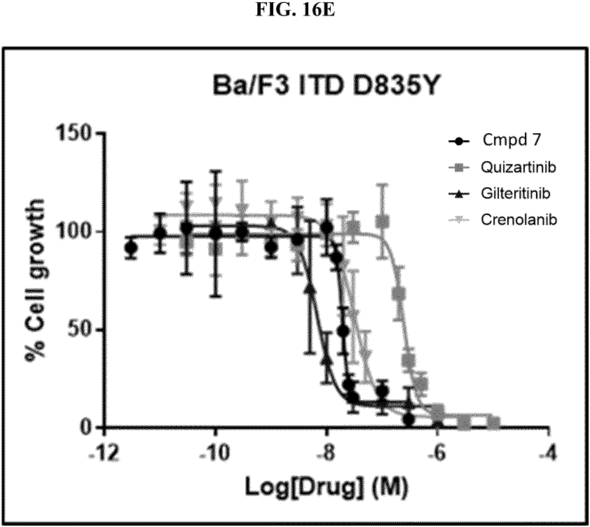

Table 3 shows cytotoxicity of Compound 7 in various heme cell lines, with the corresponding dose-response curve represented in FIG. 15. In addition, FIG. 4A. - 4D. show the cytotoxic effects of Compound 7, Quizartinib and Ibrutinib on FLT3-ITD (MV411 and MOLM-13) and FLT3-WT (NOMO-1 and KG-1) cells. Furthermore, FIGS. 16A - 16E show dose-response curves for Compound 7, Quizartinib, Gilteritinib, and Crenolanib against isogenic Ba/F3 cells transfected with FLT3 mutants, the results of which are summarized in Table 4.

Table 4

IC₅₀ of Compound 7 against isogenic Ba/F3 cells transfected with FLT3 mutants

| FLT3 Inhibitor | IC₅₀ in Transfected Ba/F3 cells (nM, n=3) | | | | |
|---|---|---|---|---|---|
| | FLT3 WT | FLT3 D835Y | FLT3 ITD | FLT3 ITD-D835Y | FLT3 ITD-F691L |
| Cmpd 7 | 11.3 | 8.8 | 0.5 | 19.3 | 10.0 |
| Quizartinib | 1956.0 | 2089.0 | 2.2 | 246.4 | 115.3 |
| Gilteritinib | 500.3 | 472.5 | 26.5 | 6.8 | 98.4 |
| Crenolanib | 2617.0 | 888.9 | 35.0 | 31.7 | 257.6 |

### Example 6: Apoptosis of MV4-11 Cells

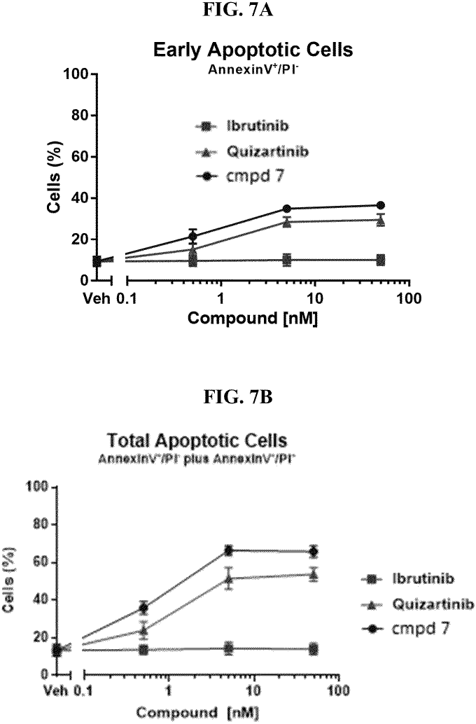

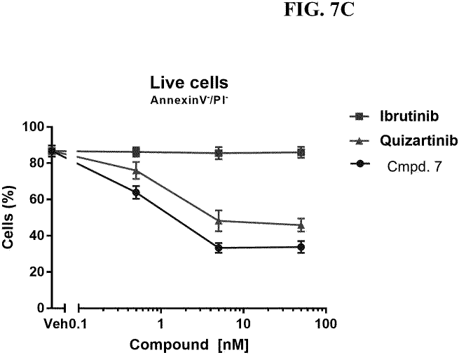

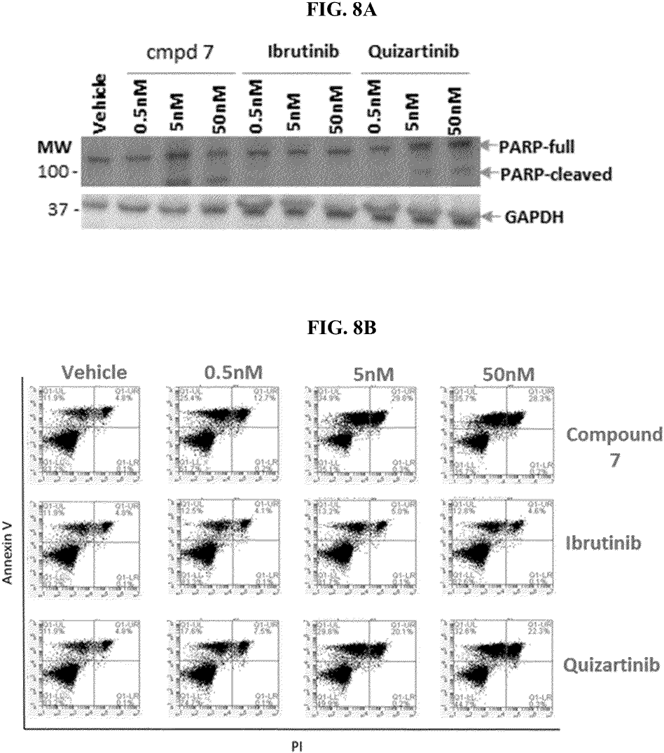

In one study, MV411 cells were independently treated with or without Compound 7, ibrutinib, or quizartinib at various concentrations for 24 hours, and the apoptotic and live cell counts were measured. Without being bound to any theory, the results in FIGS. 7A-7C and FIGS. 8A-8B demonstrate that Compound 7 induces apoptosis in MV4-11 cells.

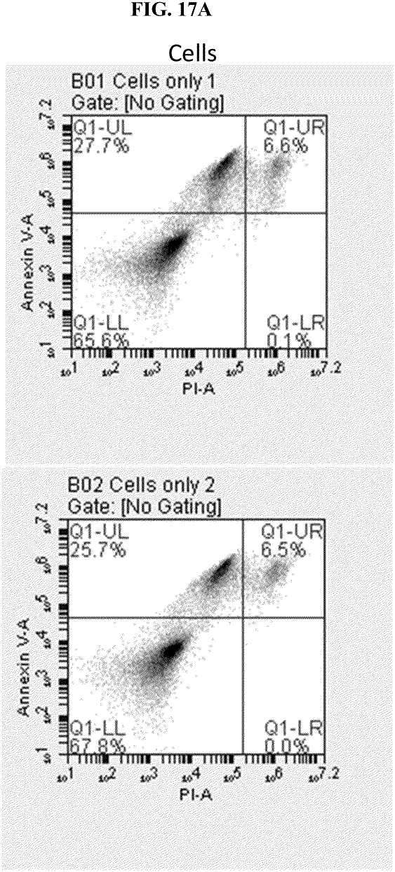

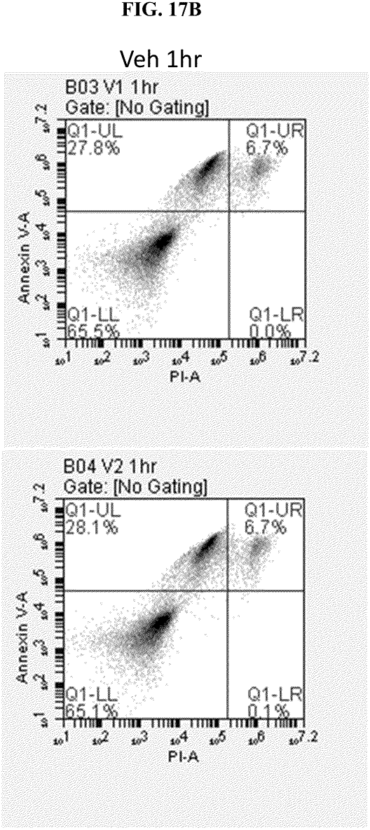

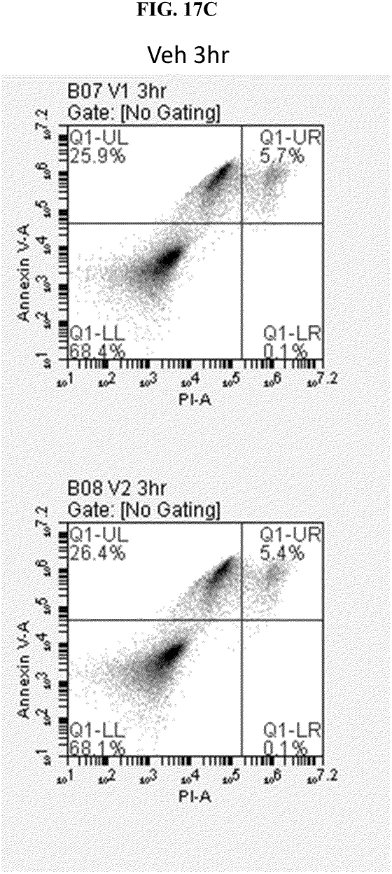

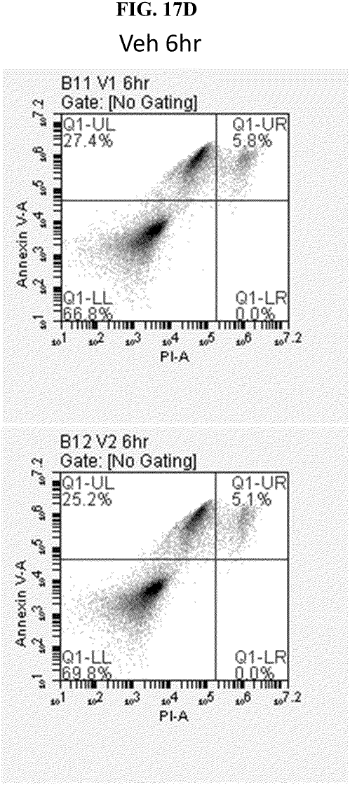

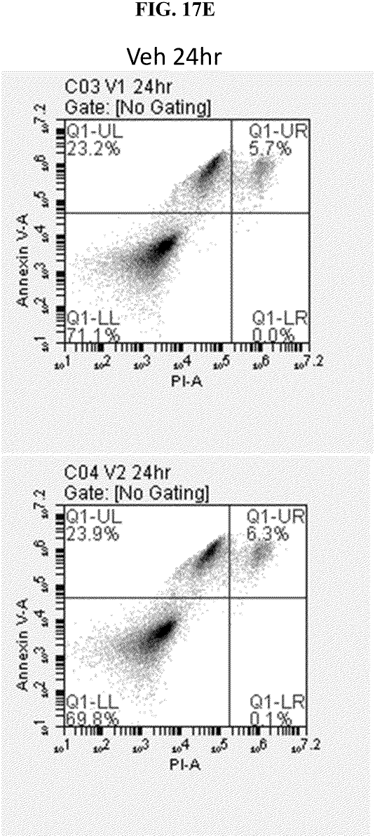

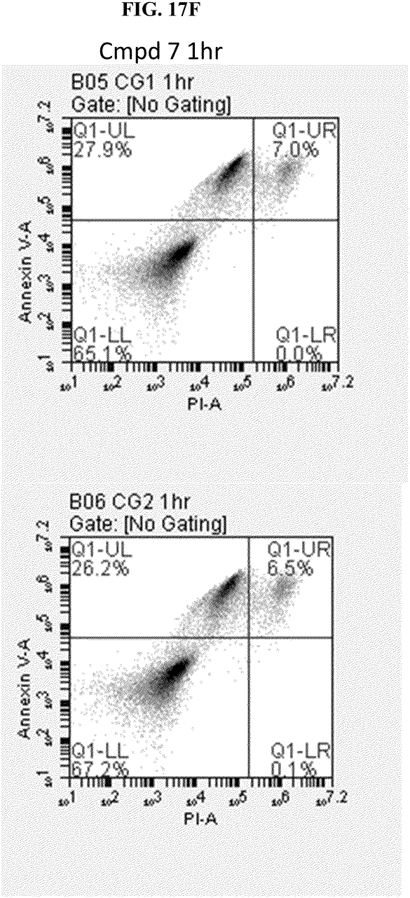

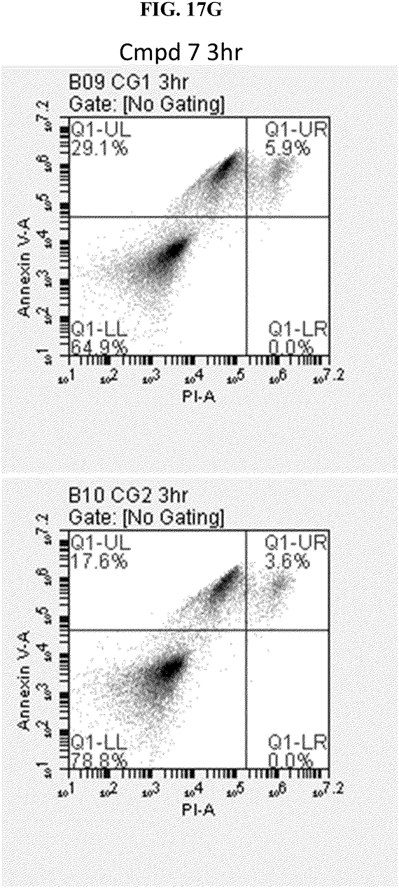

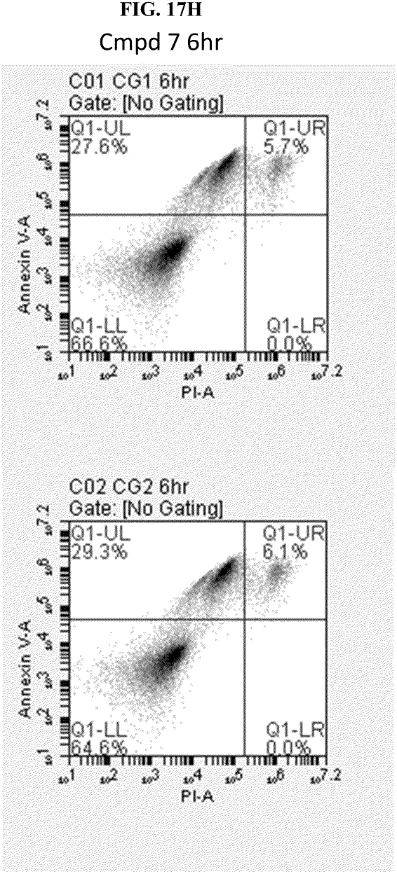

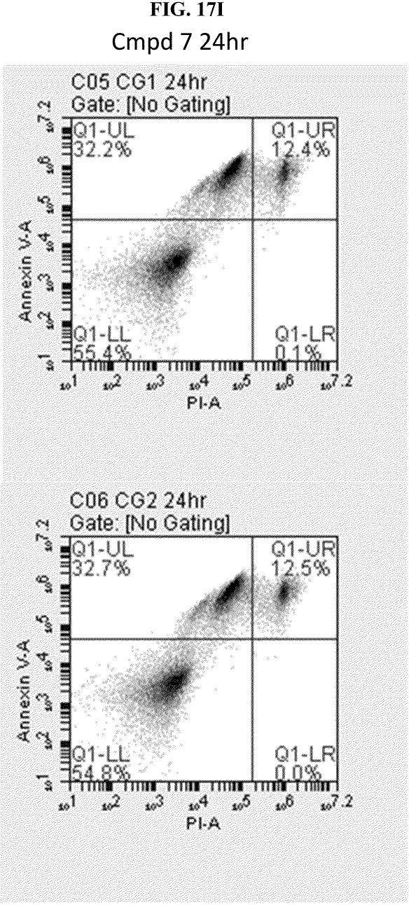

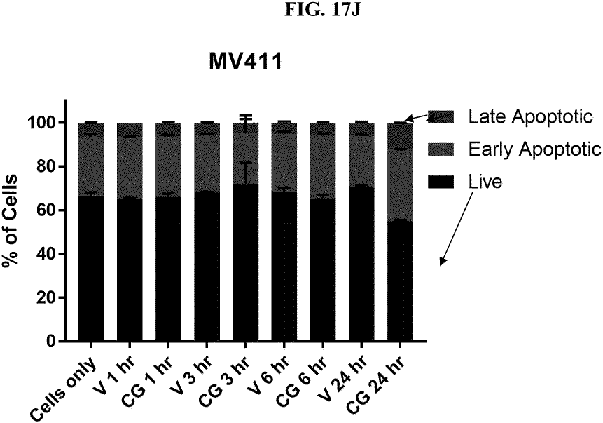

In another study, the time dependency of Compound 7 on the apoptosis of MV4-11 cells was studied, and as can be seen from FIGS. 17A-17J, Compound 7 induces apoptosis in a time dependent manner. The data presented in FIGS. 7A-7C, FIGS. 8A-8B and FIGS. 17A-17J were generated using well-known protocols for measuring apoptosis, which are readily apparent to a skilled artisan. Without being bound by any theory, an overview of the procedure can be found in, for example, Rieger et al., Modified Annexin V/Propidium Iodide Apoptosis Assay For Accurate Assessment of Cell Death, J VI. Exp. 2011; (50): 2597.

### Example 7: Pharmacokinetic Study in Rat

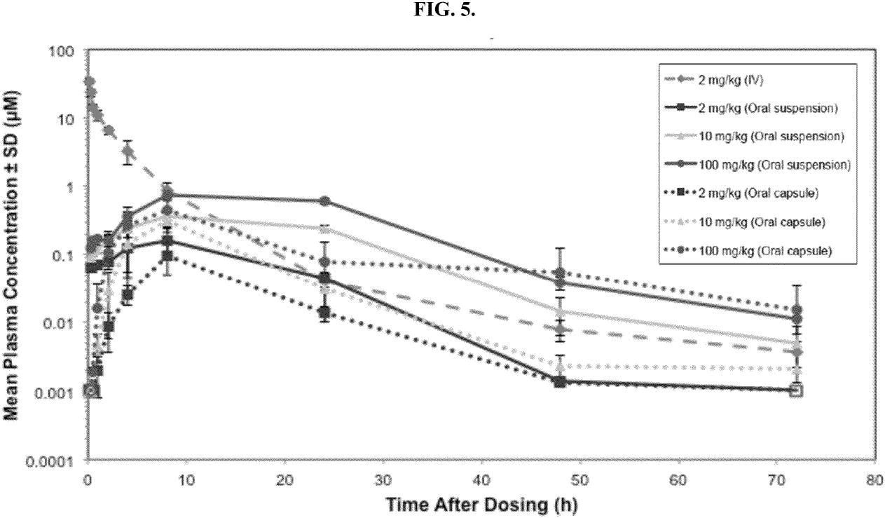

FIG. 5. demonstrates that AUC improves in a dose-dependent manner when compound 7 is administered orally. Best results are obtained for the 100 mg/kg oral suspension. Reasonable exposure was also achieved with a low dose of 2 mg/kg administered by i.v.

### Example 8: Xenografts

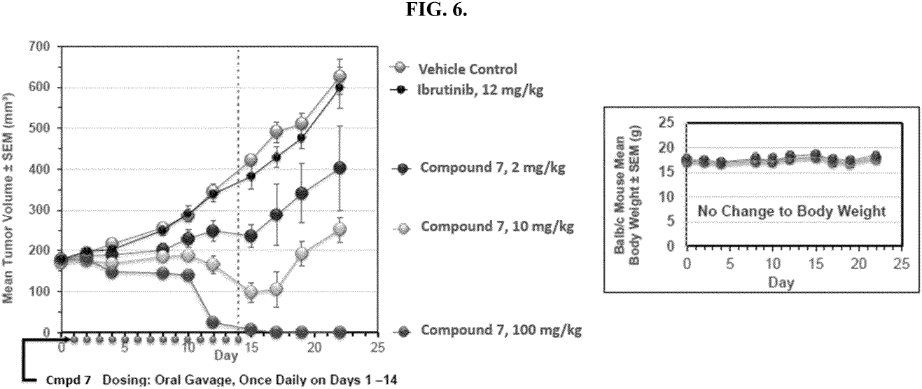

FIG. 6. demonstrates that Compound 7 reduces tumor volume with increased dose when compared to the control or Ibrutinib treatment.

Example 9: Inhibition of BTK with Compound 7

Table 5

IC₅₀ of Compound 7 in BTK

| | IC₅₀ (nM) |
|---|---|
| BTK | 8.42 |
| BTK (C481S) | 2.52 |
| BTK (E41K) | 14.53 |
| BTK (P190K) | 6.59 |

Table 5 demonstrates that Compound 7 inhibits series of BTKs.

### Example 10: Western Blot Analysis for Compound 7 in MV4-11 Cells and EOL-1 Cells

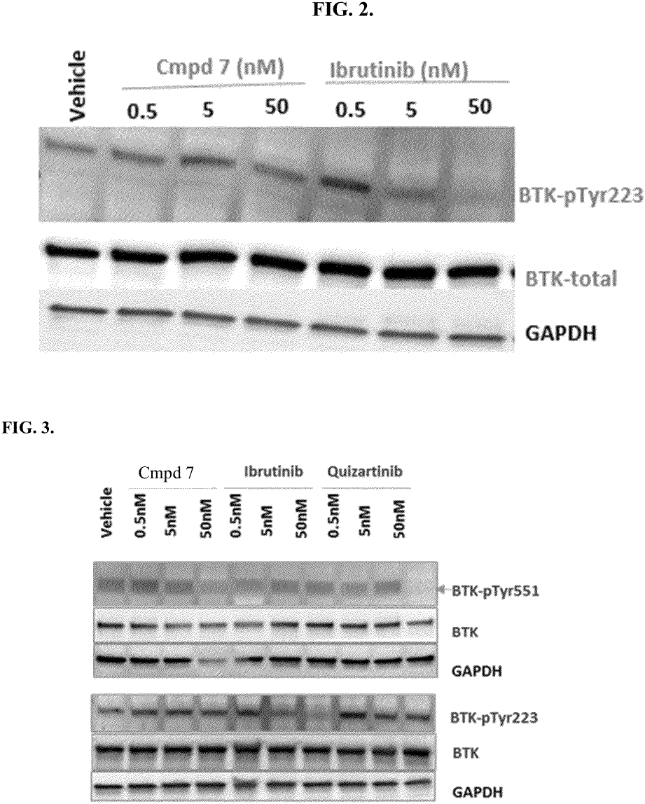

FIG. 2 and FIG. 3 shows the Western-blot analysis results. Without bound to any theory, this demonstrates that Compound 7 inhibits BTK pathway in MV4-11 cells and in EOL-1 cells.

Example 11: Comparison of Antiproliferative Activity of FLT3 WT and FLT Mutants

Table 6

Comparison of Antiproliferative Activity of Compound 7, Quizartinib and Gilteritinib in AML Cell Lines

| | Cell Lines | Characteristics | IC₅₀ (nM)² | | |
|---|---|---|---|---|---|
| | | | Compound 7 | Quizartinib | Gilteritinib |
| Murine Cells | Ba/F3-FLT3 | FLT3-WT | 9.49 | 794 | 79.52 |
| | Ba/F3-ITD | FLT3-ITD | 0.30 | 0.74 | 36.21 |
| | Ba/F3-D835G | FLT3-D835G | 0.12 | 1.27 | 28.08 |
| | Ba/F3-D835Y | FLT3-D835Y | 8.26 | 776.21 | 372.68 |
| | Ba/F3-ITD+691 | ITD+F691L | 0.43 | 33.60 | 119.45 |
| | BalF3-ITD+842 | ITD+Y842C | 0.73 | 6.73 | 44.74 |
| | Ba/F3-ITD+D835Y | FLT3-ITD+D835Y | 9.72 | 85.31 | 10.59 |
| | Ba/F3-ITD+D835H | FLT3-ITD+D835H | 6.74 | 9.02 | 10.43 |
| Human Cells | MOLM13 | FLT3-ITD, t(9:11) | 0.82 | 5.21 | 35.79 |
| | MOLM14 | FLT3-ITD, t(9:11) | 0.92 | 0.67 | 58.83 |
| | MV4-11 | FLT3-ITD, t(4:11) | 0.17 | 1.39 | 175.66 |
| | THP-1 | FLT3-WT, t(9:11) | 3.88 | >10000 | 2301 |
| | Kasumi-1 | FLT3-WT | 21.99 | 29.7 | 2233 |

The half maximal inhibitor concentrations (IC₅₀S) were generated by 72 h treatment (Gilteritinib was that from 48 h).

### Example 12: Ex Vivo Assay for Patient Sensitivity to Compound 7

85 Patients with a diagnosis of acute myeloid leukemia (AML), 15 patients with myelodysplastic syndrome/myeloproliferative neoplasms (MDS/MPN), 18 patients with acute lymphoblastic leukemia (ALL), and 56 patients with chronic lymphocytic leukemia (CLL) were evaluated for sensitivity to the multi-kinase inhibitor, Compound 7, using an ex vivo assay. Sensitivity to Compound 7 was evaluated across a concentration range from 10 nM to 10 µM.Cell viability was assessed using a colorimetric, tetrazolium-based MTS assay after a 3-day culture period, and IC₅₀ values were calculated as a measure of drug sensitivity.

Across the four general subtypes of hematologic malignancies in the dataset, there is broad sensitivity to Compound 7. A majority of AML cases showed sensitivity to Compound 7, with 51/85 (60%) cases exhibiting an IC₅₀ of less than 0.1 µM.Sensitivity of other subtypes (MDS/MPN, ALL CLL) showed comparable or slightly lower levels of sensitivity (40-60%, see Table 7). FLT3 mutational status is known for 38 AML patient samples in the dataset: 30 samples are WT, 8 samples are ITD+, 0 samples have a point mutation. Analysis of Compound 7 sensitivity in this subset reveals a trend of enhanced sensitivity in FLT3-ITD+ samples; however, a larger sample size will be necessary to sufficiently power statistical analyses.

Table 7

Compound 7 sensitivity in patients

| Diagnosis Type | N evaluated | % samples with IC₅₀ < 0.1 µM |
|---|---|---|
| AML | 85 | 60 (51/85) |
| MDS/MPN | 15 | 53 (8/15) |
| ALL | 18 | 61 (11/18) |
| CLL | 56 | 41 (23/56) |

Compound 7 exhibits broad and potent activity against AML as well as other hematologic malignancy subtypes. Preliminary analyses show a trend of greater Compound 7 sensitivity in FLT3 mutant AML cases compared with FLT3 wild type; however, ongoing accrual of additional patient samples may be required to sufficiently power a statistical association of Compound 7 sensitivity with FLT3 mutational status. In sum, without bound by any theory, preclinical analyses of Compound 7 against primary hematologic malignancy patient samples show evidence of broad drug activity in AML and other disease subtypes and support further development of this agent for hematologic malignancies.

### Example 13. Compound 7’s Effect on Cell-Cycle Dysregulation

The effects of Compound 7 on various aspects of the cell-cycle were examined.

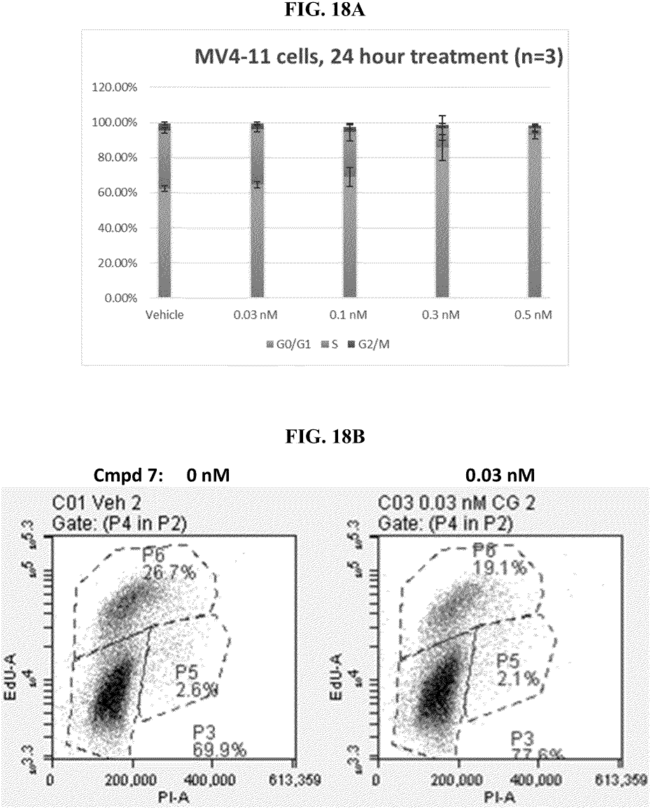

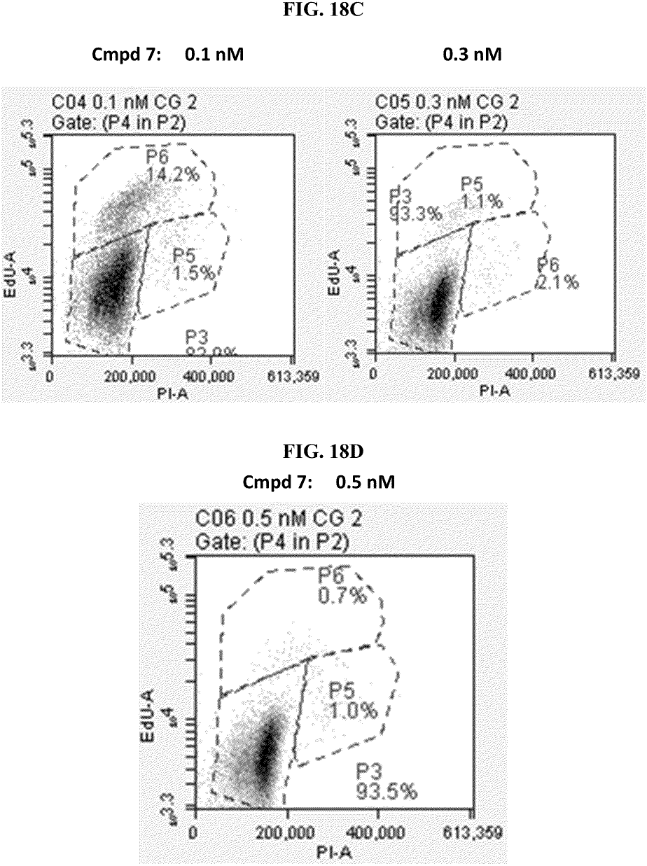

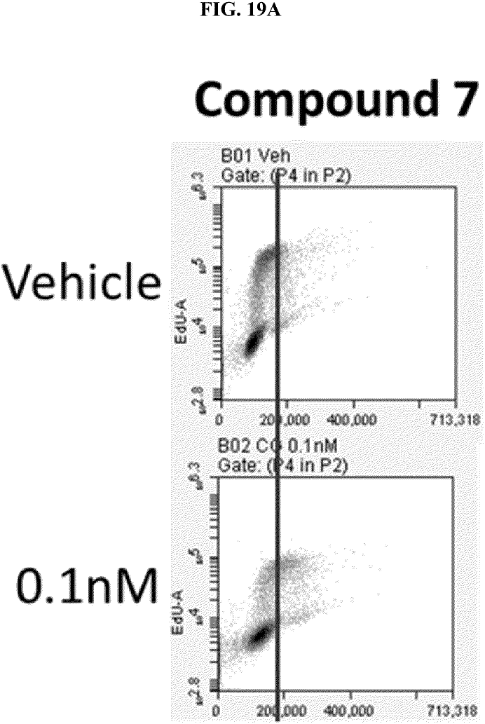

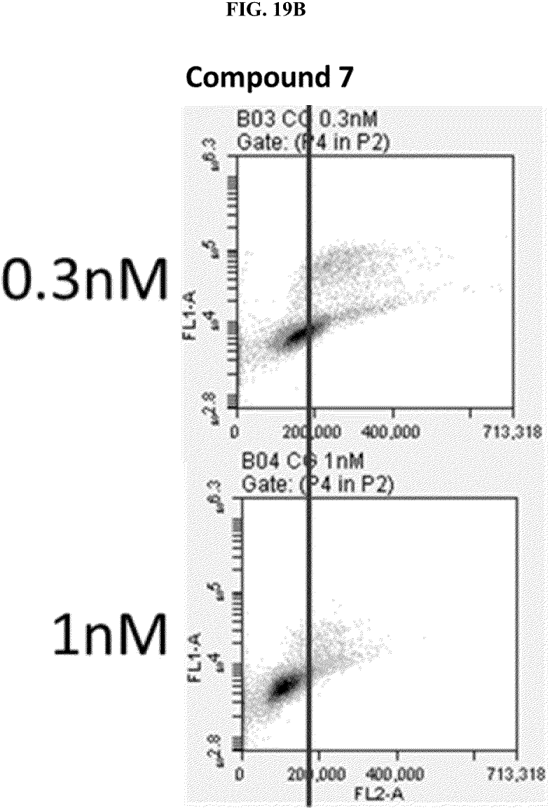

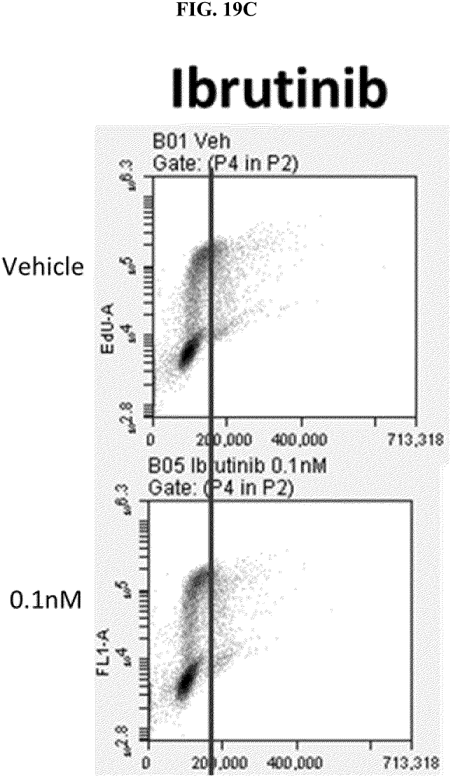

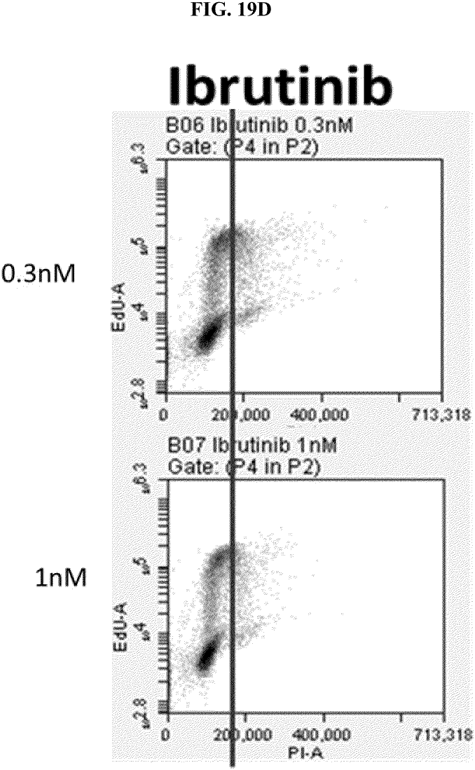

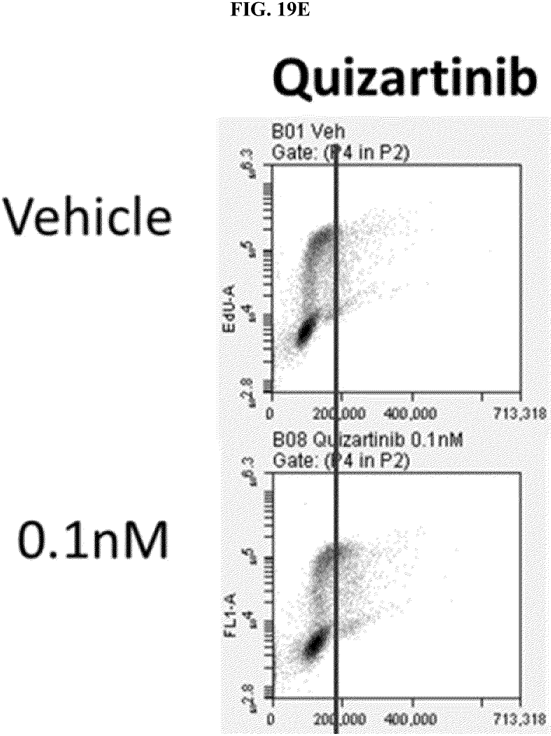

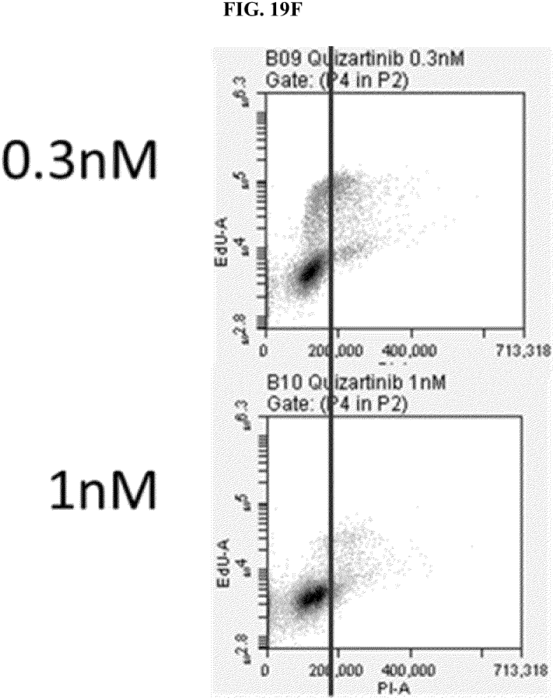

In one experiment, Compound 7 was found to induce G0/G1 cell-cycle arrest in MV411 cells in a dose-dependent fashion (FIGS. 18A - 18D). In another experiment, Compound 7 was found to induce G0/G1 cell-cycle arrest in MOLM-13 cells in a dose-dependent fashion (FIGS. 19A - 19F).

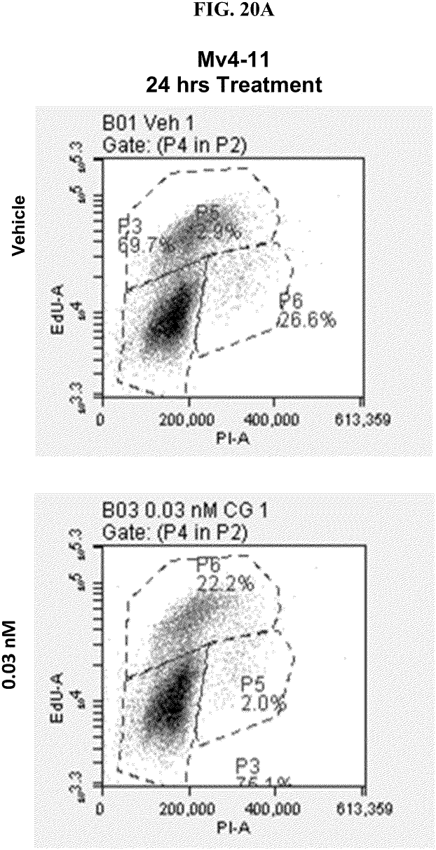

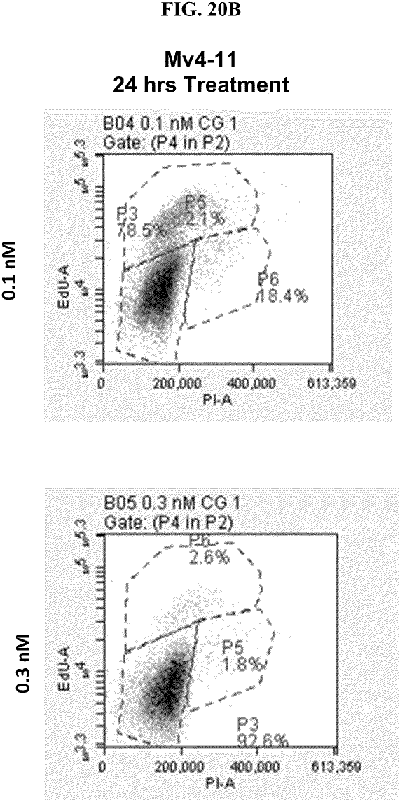

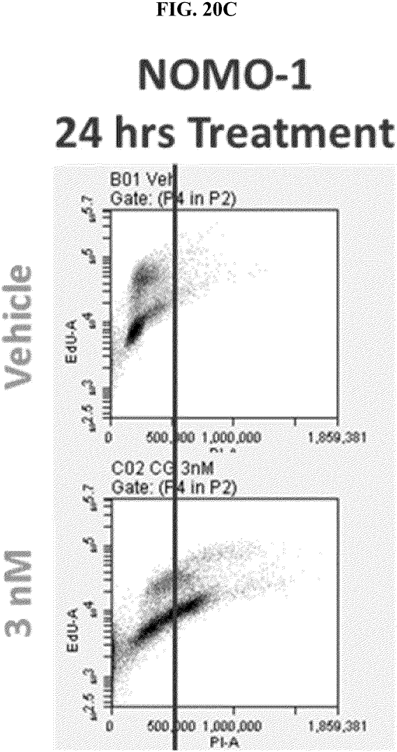

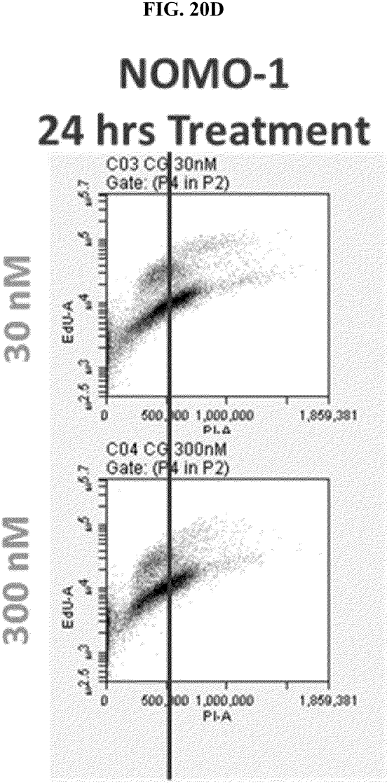

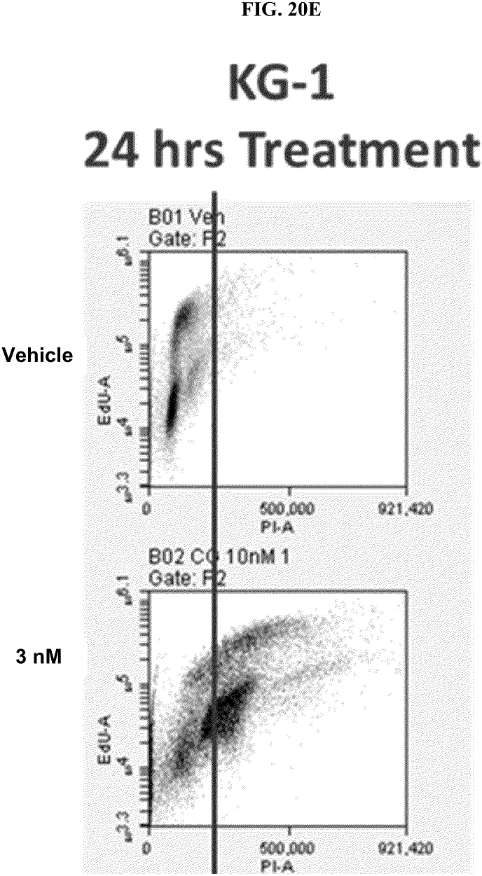

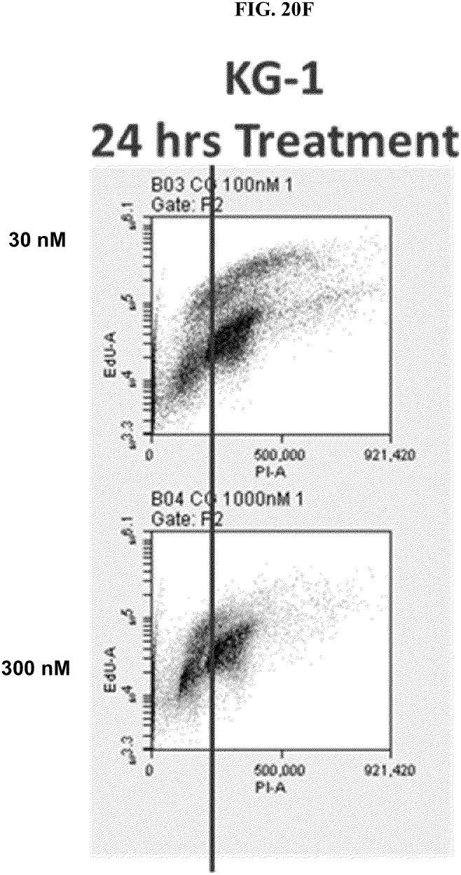

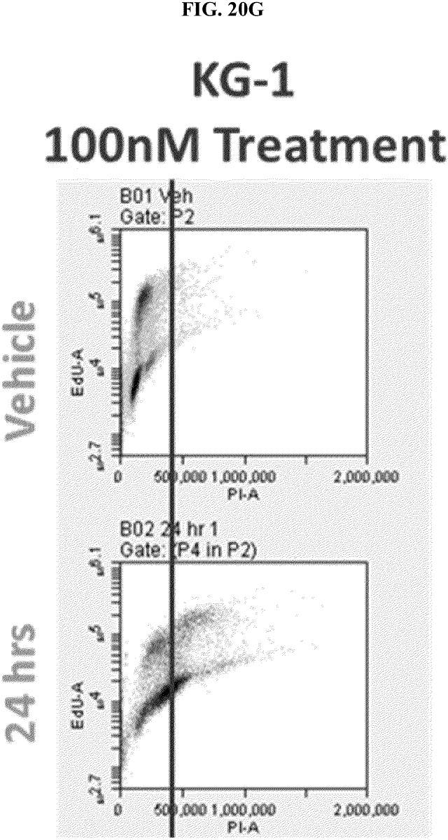

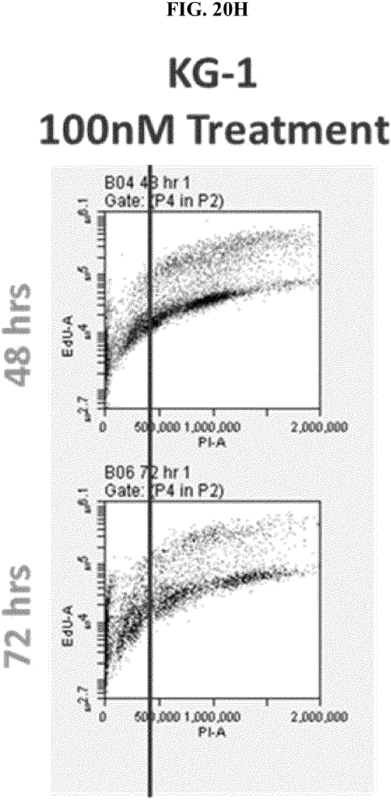

In another experiment, Compound 7 was found to induce polyploidies in various heme cell lines (FIGS. 20A-20H). MV4-11, NOMO-1, and KG-1 cells were treated at increasing concentrations of Compound 7 for 24 hours, and the increase in DNA content or polploidy phenotype was assessed using EdU and PI staining followed by FACS analysis using the BD Accuri C6 flow.

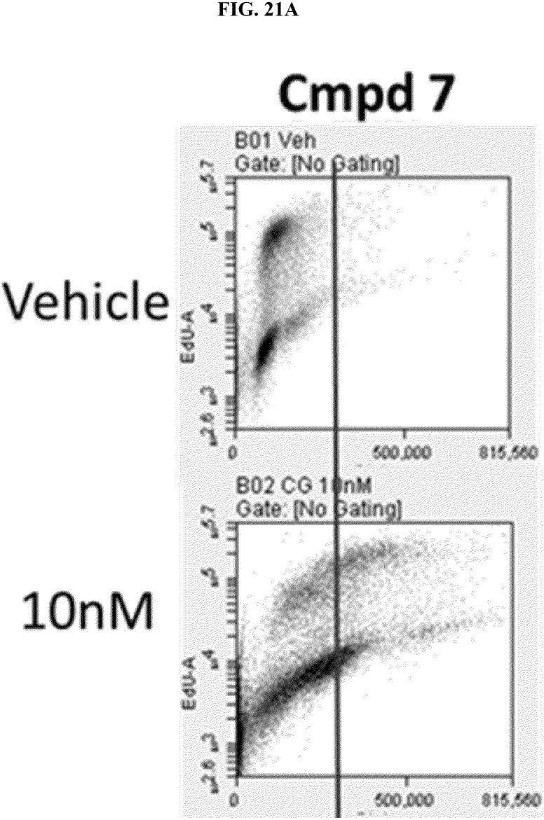

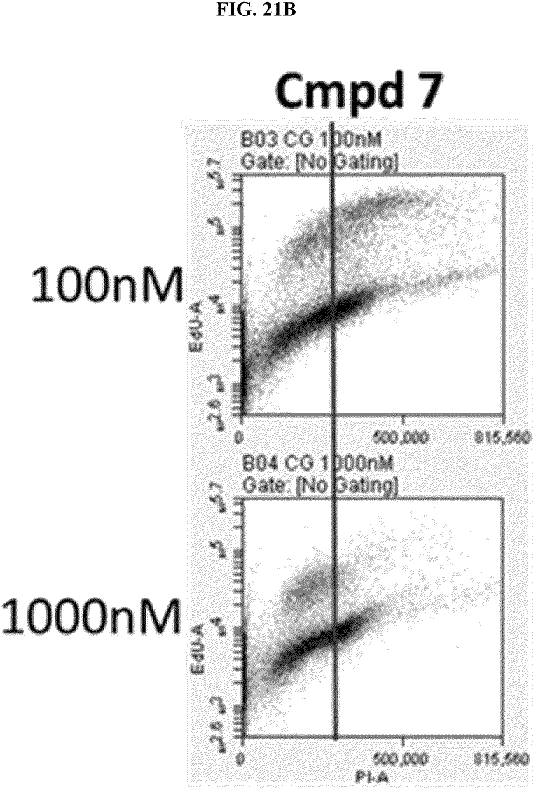

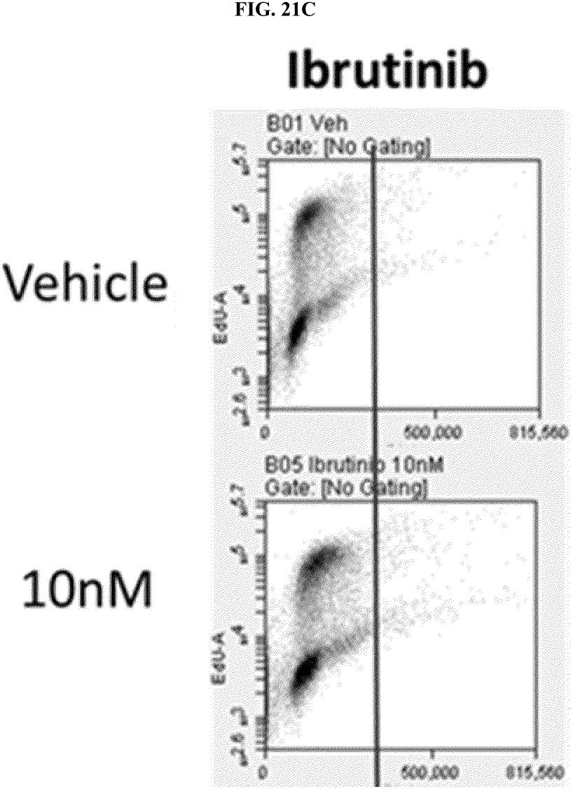

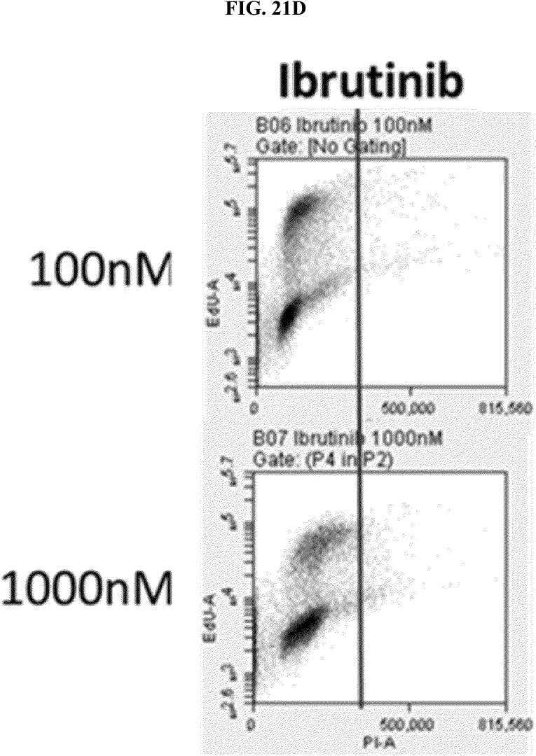

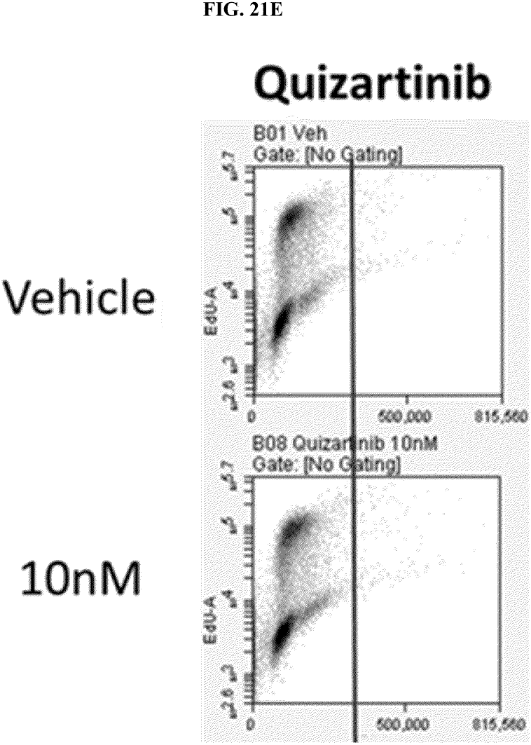

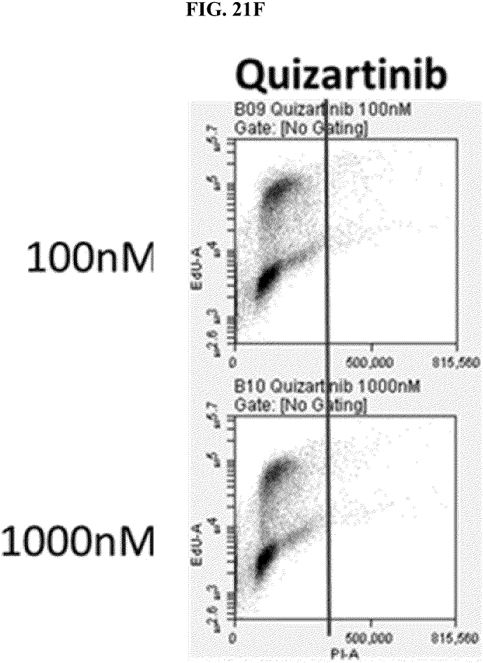

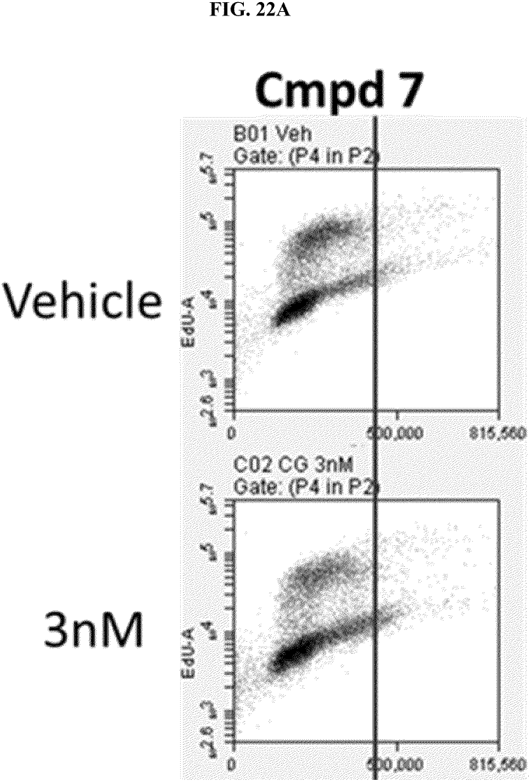

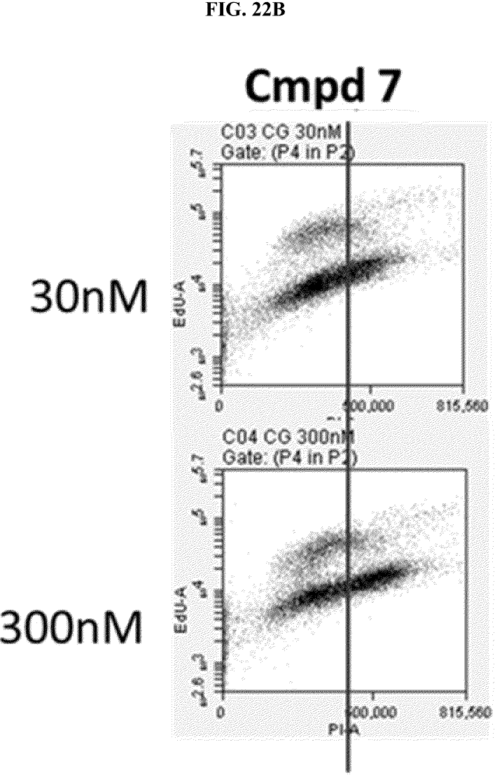

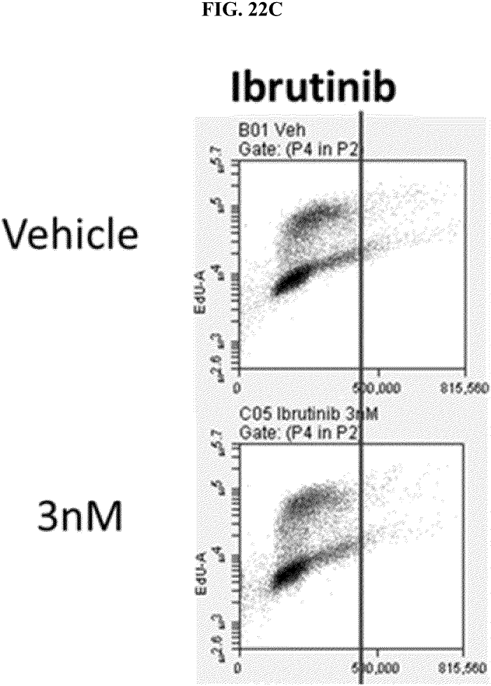

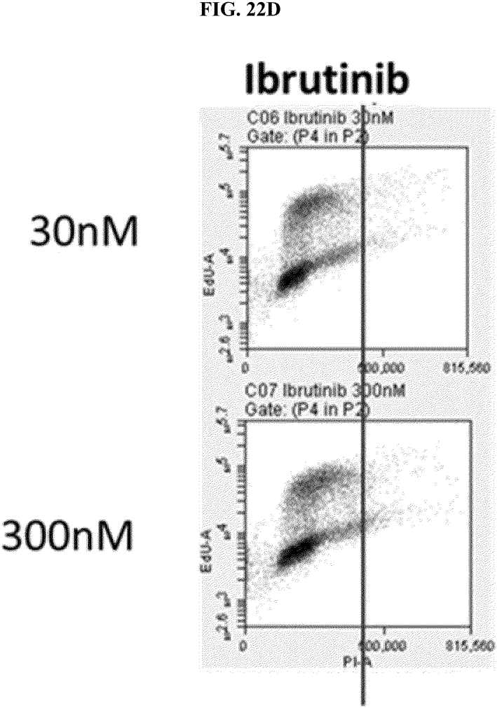

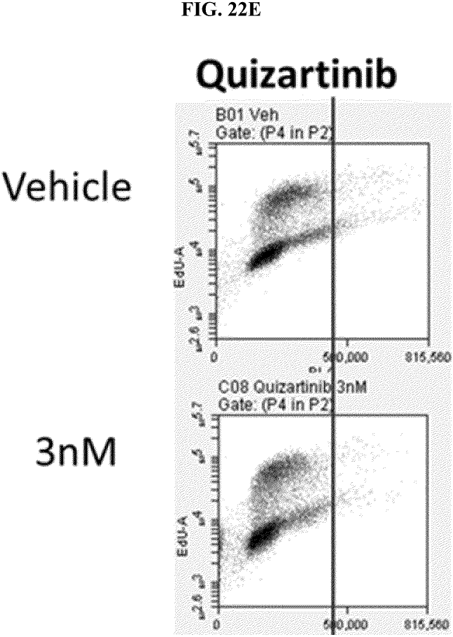

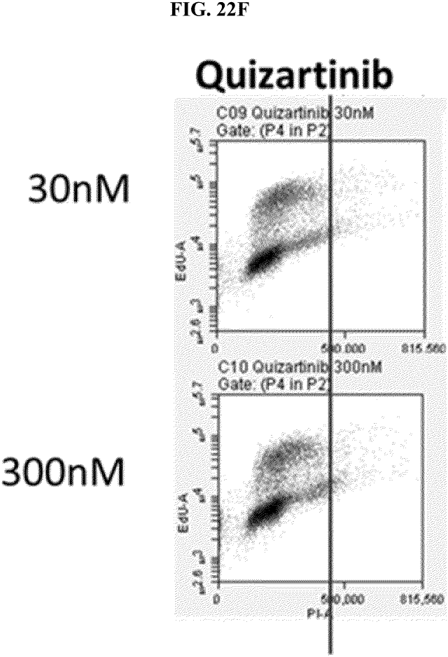

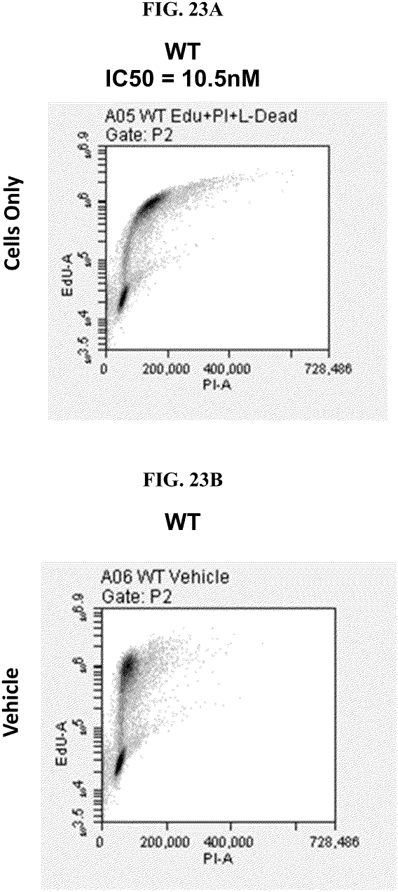

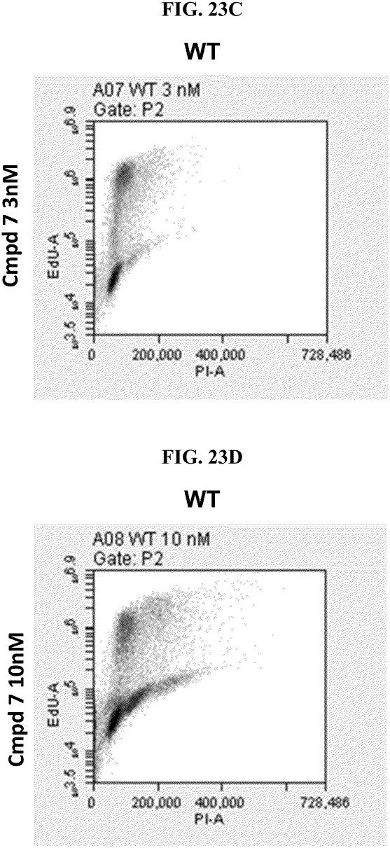

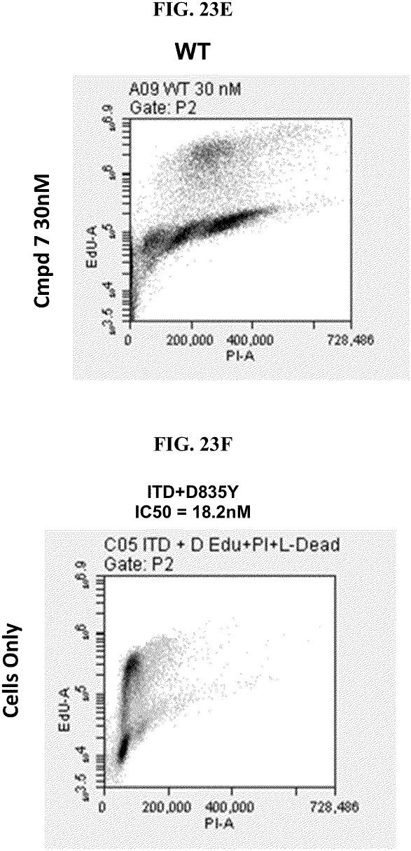

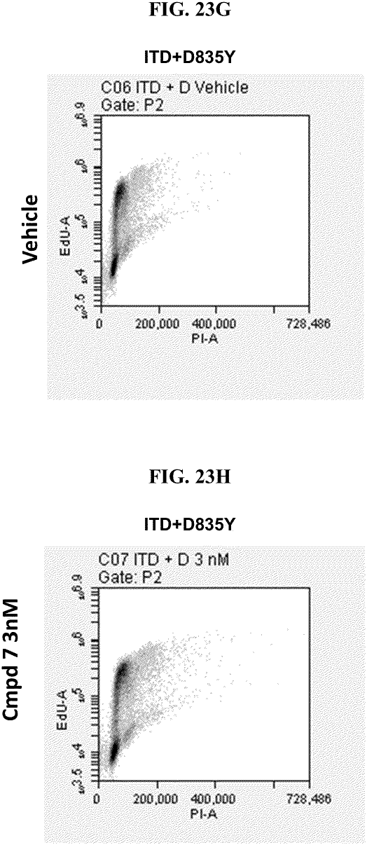

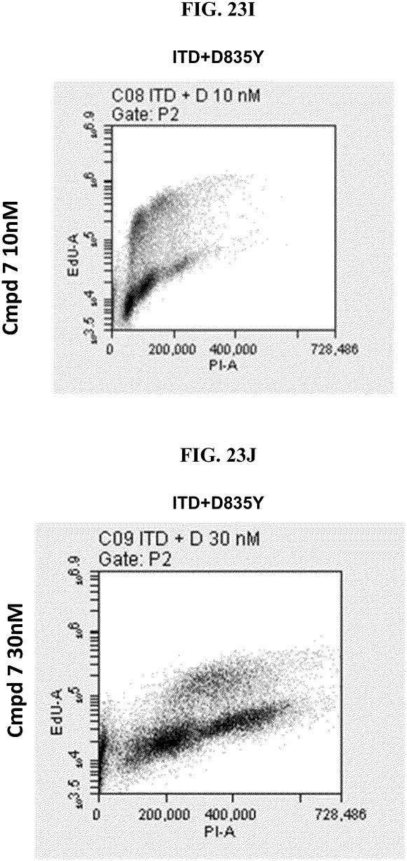

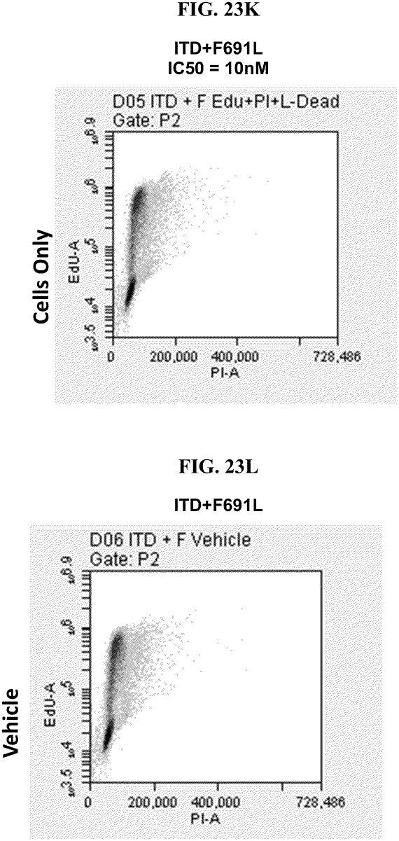

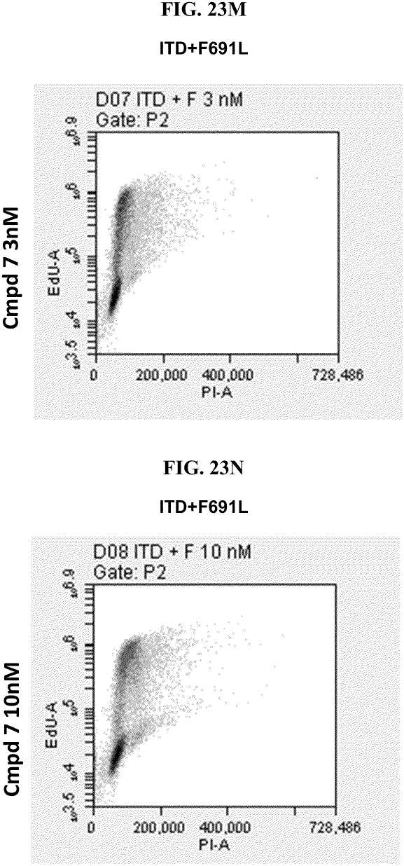

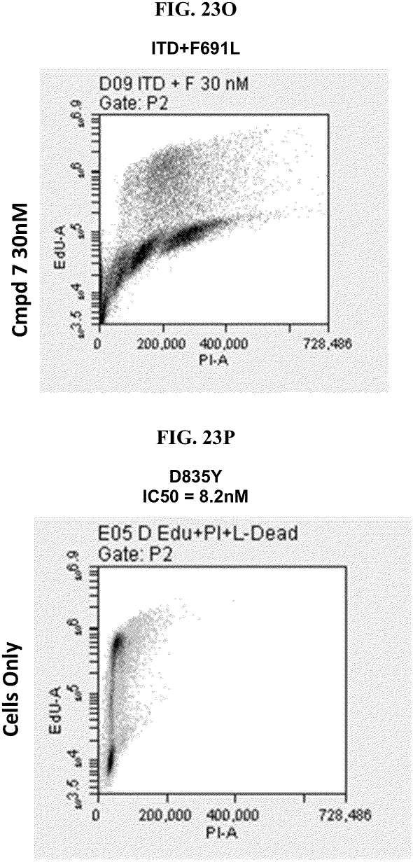

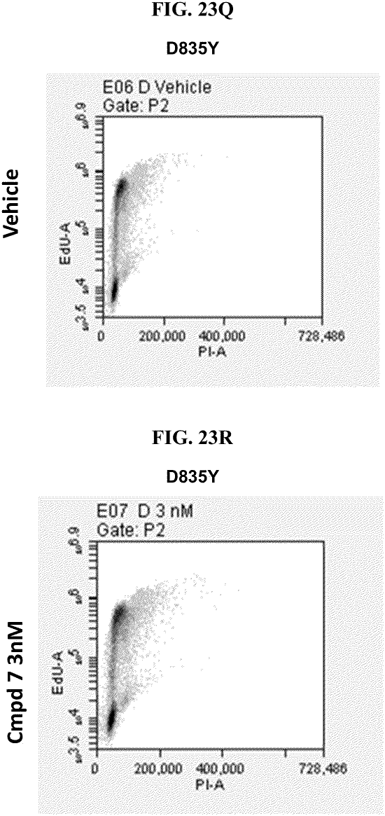

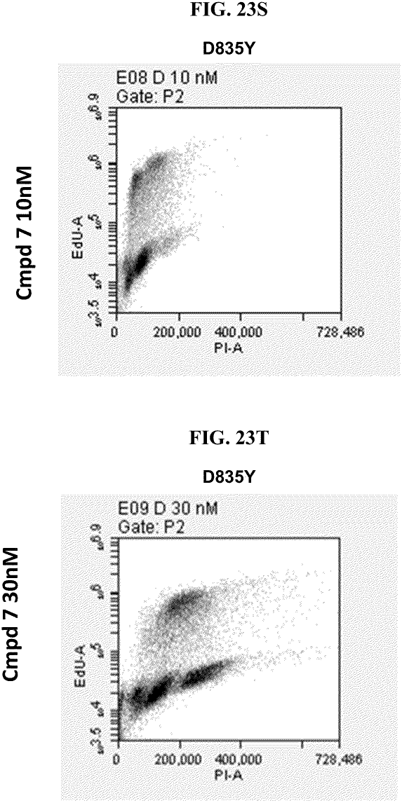

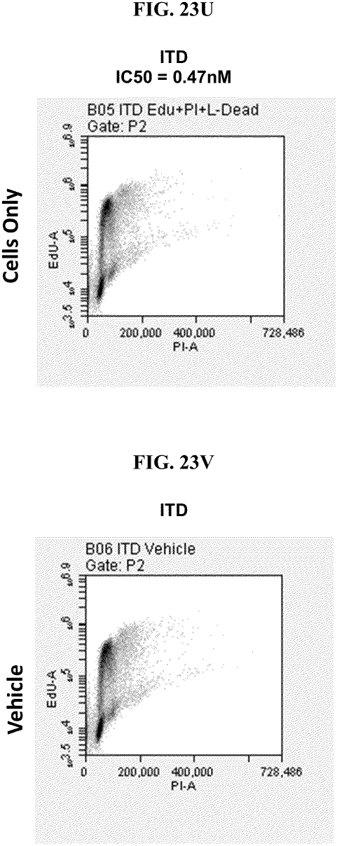

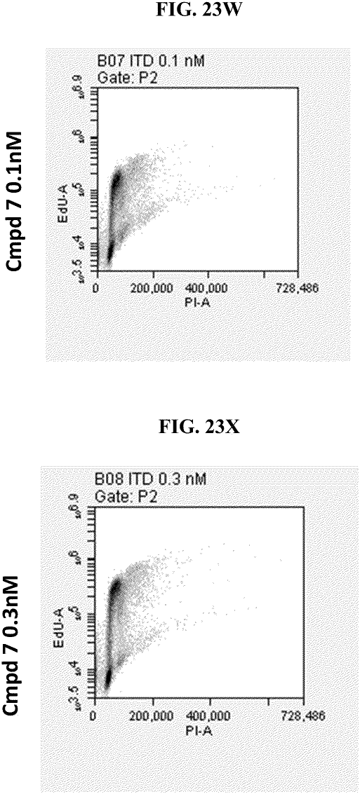

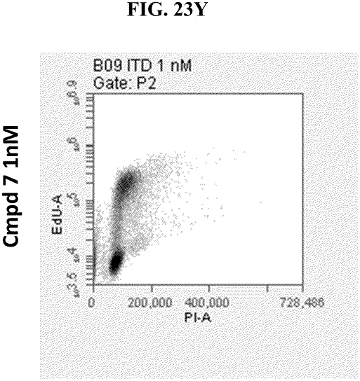

In another experiment, Compound 7 was found to induce cell-cycle dysregulation in KG-1 cells (FIGS. 21A - 21F), NOMO-1 cells (FIGS. 22A - 22F), and isogenic BA/F3 cells with FLT3 mutations (FIGS. 23A - 23Y) in a dose-dependent fashion.

### Example 14. Compound 7’s Inhibitory Effects on Cell Signaling in Heme Cells

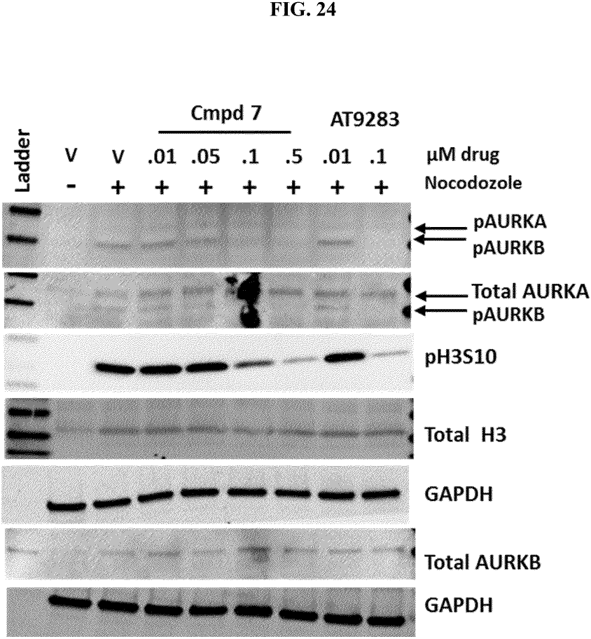

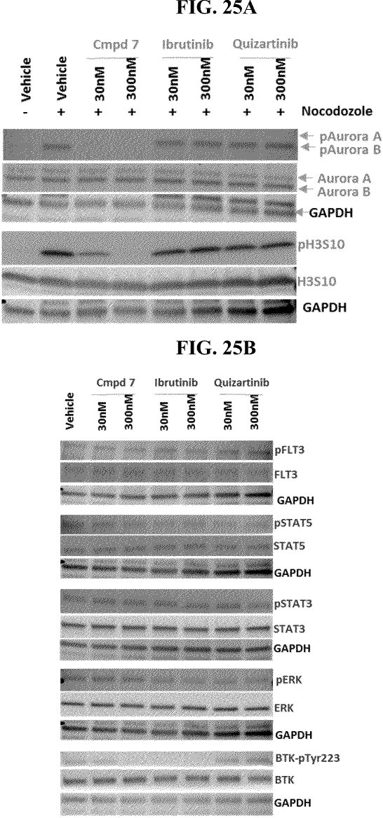

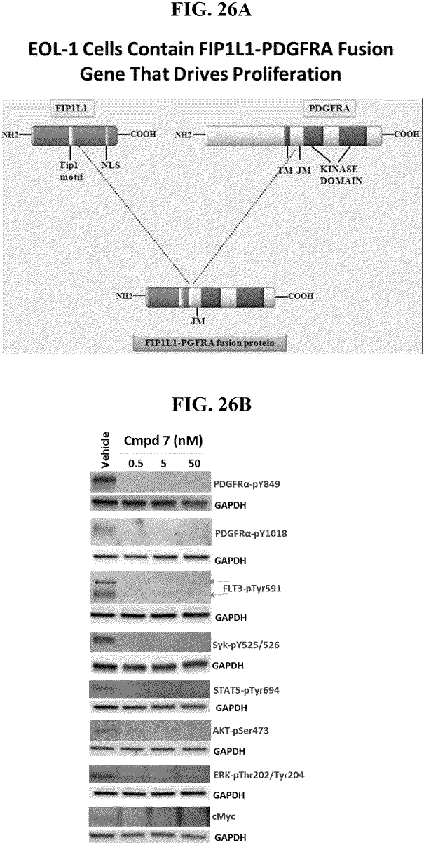

FIG. 24 and FIGS. 25A - 25B show that Compound 7 inhibits Aurora kinase activity and signaling in MV4-11 and FLT3 WT cells (KG-1), respectively. This is in comparition to a AT9283, an Aurora kinase inhibitor (FIG. 24) and kinase inhibitors ibrutinib and quizartinib (FIGS. 25A - 25B). FIGS. 26A - 26B shows that Compound 7 inhibits PDGFRA and FLT3 (WT) signaling in EOL-1 cells. Without being bound by any theory these figures show that Compound 7 acts as a strong inhibitor of various cell signaling pathways in Heme cell lines.

### Example 15: Comparative Potency of Compound 7 Against BTK

In addition to Compound 7’s potency against wild-type BTK, it also exhibits nM potency against the BTK-C481S mutant, as shown in Table 8. Compound 7 has a potency of 5.0 nM against wild-type BTK, which is equivalent to the potency of acalabrutinib. Even more surprisingly, Compound 7 is most effective against BTK-C481S, which is resistant to ibrutinib and acalabrutinib. Compound 7 is also extremely potent against ITK, a key target which is believed to contribute to the efficacy of ibrutinib. Furthermore, Compound 7 has no inhibition of EGFR, which suggests that there is a reduced likelihood of complications such as rashes and diarrhea.

Table 8

Inhibition of various compounds against BTK

| | | | | | BTK IC50 (nM) | | Key Off-Targets | |
|---|---|---|---|---|---|---|---|---|
| | Company | Indications | Status | Binding | WT | C481S | ITK | EGFR |
| Compound 7 | Aptose | Heme | PC | Non-Covalent | 5.0 | 2.5 | 4.3 | >1000 |
| Ibrutinib | Abbvie | CLL, MCL, WM | Mkt | Covalent | 0.5 | x | 10.7 | 5.6 |
| Acalabrutinib | AZ/Acerta | Oncology -H/S | P3 | Covalent | 5.1 | x | >1000 | >1000 |
| BGB-3111 | Beigene | Heme | P1 | Covalent | 0.2 | x | 30.0 | - |
| GS-4059 | Gilead/Ono | Heme | P1 | Covalent | 2.2 | x | - | - |
| CC-292 | Celgene | Heme | P1 | Covalent | <0.5 | x | - | - |
| SNS-062 | Sunesis | Heme | P1 | Non-Covalent | 0.4 | 4.5 | 49.0 | - |

### Example 16: Compound 7 Inhibits Wild-Type and C481S Mutant Form of BTK

HEK293T cells were transiently transfected with wild-type BTK or C481S BTK. The transfected cells were treated with or without Compound 7 (0.5 and 1.0 µM) for six hours. This was performed in triplicate and the results were analyzed by Western Blot analysis.

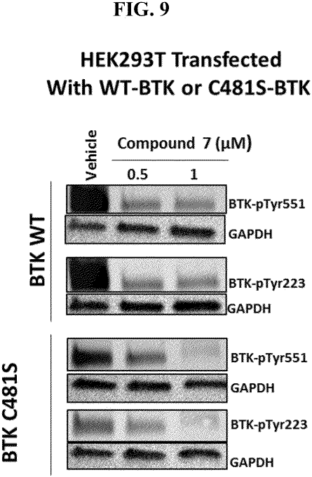

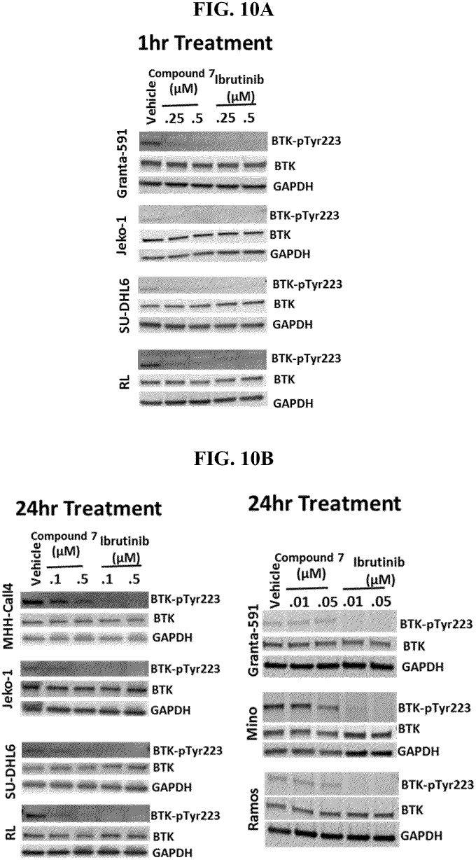

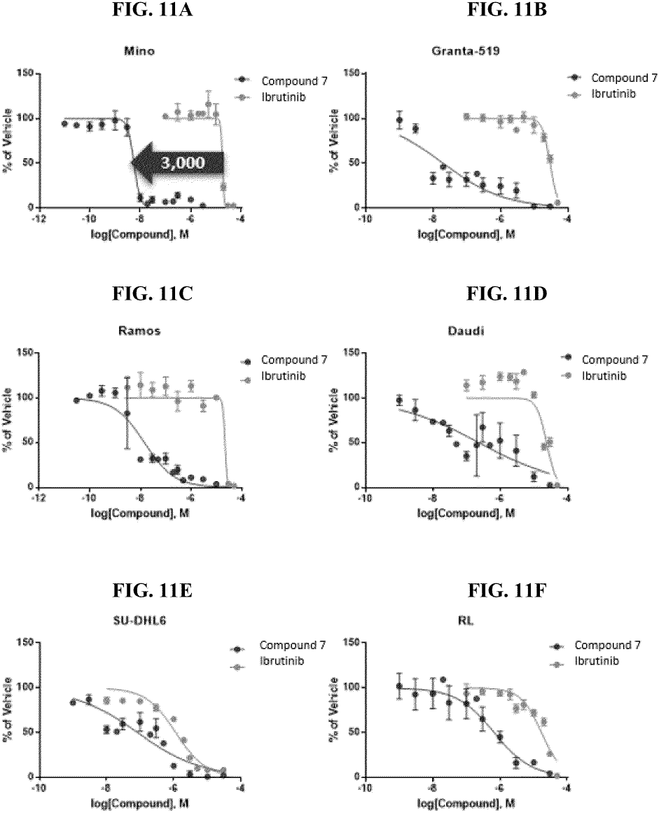

As evidenced by the reduction in the phosphorylated forms of the enzymes in FIG. 9, Compound 7 inhibits both wild-type and the C481S mutant form of BTK at concentrations of both 0.5 and 1.0 µM.However, when compared to ibrutinib, Compound 7 is observed to inhibit BTK to a lesser extent than ibrutinib, suggesting a different mechanistic pathway (FIGS. 10A -10B).

### Example 17: Cytotoxicity of Compound 7 on B-Cell Malignancy Cell Lines

Cells were seeded in a 96 well plate and treated with either vehicle (DMSO) or compound 7 at 10 different concentrations for 3 days at 37° C. and 5% CO₂. Cell viability was assessed using CellTiter 96 AQᵤₑₒᵤₛ one solution (MTS Promega Cat#G3581), and IC₅₀ values calculated using GraphPad Prism 7 software.

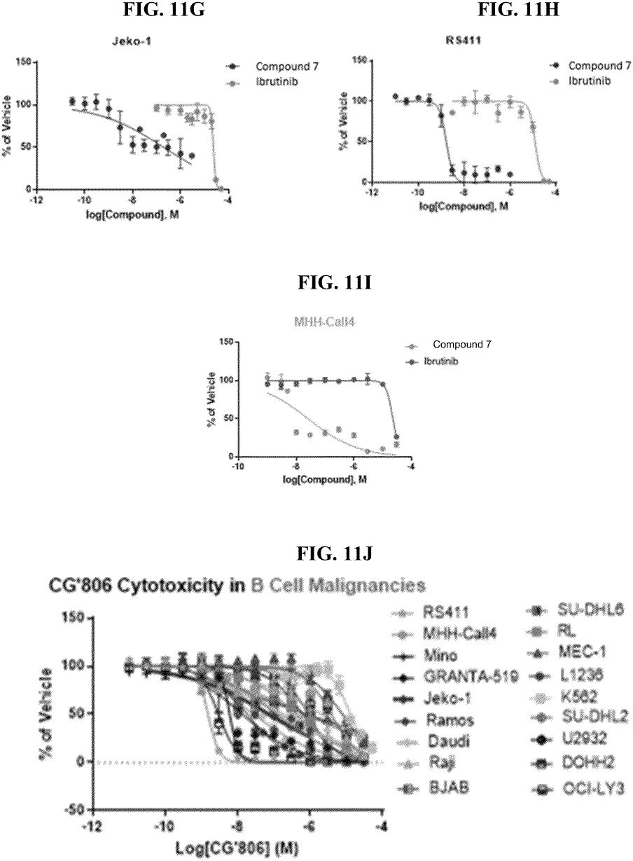

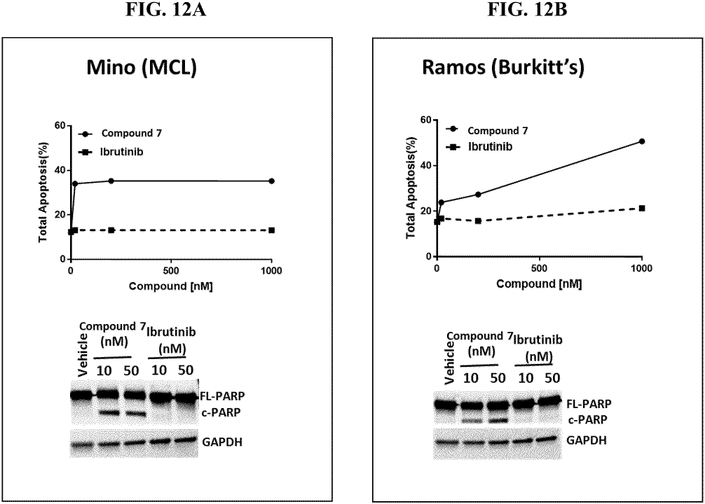

Table 9 shows cytotoxicity of Compound 7 in various B-cell malignancy cell lines. In addition, FIGS. 11A - 11J shows the dose-response curves for Compound 7 and ibrutinib on the cell lines of Table 9.

Table 9

Cytotoxicity of Compound 7

| B Cell Malignancy | Cell lines | IC50(µM) |
|---|---|---|
| | | Mean |
| B-ALL | RS411 | 0.001508 |
| | MHH-Call4 | 0.027800 |
| FL | DOHH2 | 0.002937 |
| MCL | Mino | 0.005976 |
| | GRANTA-519 | 0.013590 |
| | Jeko-1 | 0.073770 |
| Burkitt’s | Ramos | 0.018410 |
| | Daudi | 0.166000 |
| | Raji | 2.031000 |
| GCB-DLBCL | SU-DHL6 | 0.121600 |
| | RL | 0.426200 |
| | BJAB | 0.882000 |
| ABC-DLBCL | U2932 | 0.632300 |
| | SU-DHL2 | 0.744400 |
| | OCI-LY3 | 0.830500 |
| CLL | MEC-1 | 3.804000 |
| HL | L1236 | 7.818 |
| CML | K562 | 12.940000 |

### Example 18: Compound 7 Induces Apoptosis in B-Cell Malignancies

Mechanistic studies were undertaken to determine the mechanism of action for Compound 7. The apoptotic state of treated cells was determined by staining with annexin V and propidium iodide (PI), then analysis with the BD Accuri C6 flow cytometer, whereby live cells are annexin V / PI negative, early apoptotic cells are annexin V positive and late apoptotic cells are annexin V and PI positive. In addition production of cleaved PARP, a classic apoptotic marker, was assayed by western blotting with specific antibodies.

As shown in FIGS. 12A - 12B, Compound 7 induces cellular apoptosis in B-cell malignancies. Both Mino and Ramos cell lines were treated with increasing concentrations of Compound 7 and ibrutinib. Compound 7 induced apoptosis more effectively than ibrutinib at all concentrations tested. The phosphorylation pattern in the respective western blots confirm this increase in apoptosis.

### Example 19: Compound 7 Inhibits Aurora Kinases and BTK in B-Cell Malignancies

Inhibition of Aurora kinase activity was measured by western blotting for phosphorylation levels of Aurora kinases or and down-stream targets and by cell cycle and DNA content analysis. Whole cell extracts from Compound 7 treated cells were resolved by gel electrophoresis and transferred to nitrocellulose membranes and inhibition of Aurora kinase A/B and H3S10 phosphorylation was detected with specific antibodies. DNA synthesis and cell cycle phase was assessed by staining compound 7 of vehicle treated cells with 5-ethynyl-2′ deoxyuridine (Edu) Alexa Fluor 488 and PI.

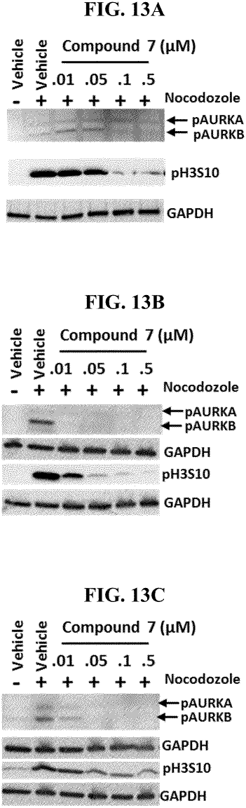

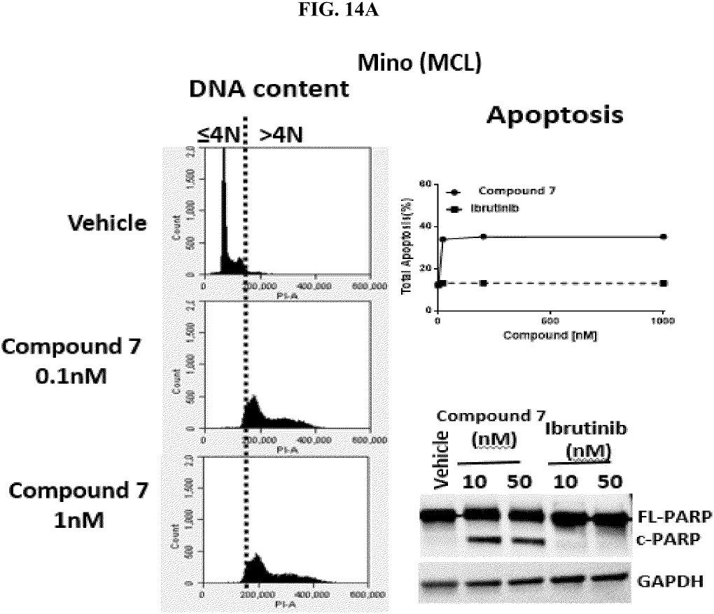

While Compound 7 is more cytotoxic towards B-cell cancer cells than ibrutinib, it is a less active BTK inhibitor than ibrutinib (FIGS. 10A - 10B). To better understand Compound 7’s high potency, its inhibitory effects on Aurora kinase was examined and it was found to be effective as an Aurora Kinase inhibitor (FIGS. 13A - 13C) as confirmed by the phosphorylation patterns in the Wester Blot at increased concentration of Compound 7. Without being bound by any particular theory, Compound 7’s high cytotoxicity in B-cell cancer cells is believed to be attributed in part to its multi-kinase pathway inhibitory profile.

### Example 20: Compound 7 Induces Polyploidy in B-Cell Malignancies Followed by Apoptosis

Further mechanistic studies were undertaken to fully elucidate Compound 7’s potent cytotoxicity. B-cell lines were treated with vehicle or Compound 7 for 24-72 hours and the increase in DNA content or polyploidy phenotype was assessed using Edu and PI staining followed by FACS analysis using the BD Accuri C6 flow cytometer.

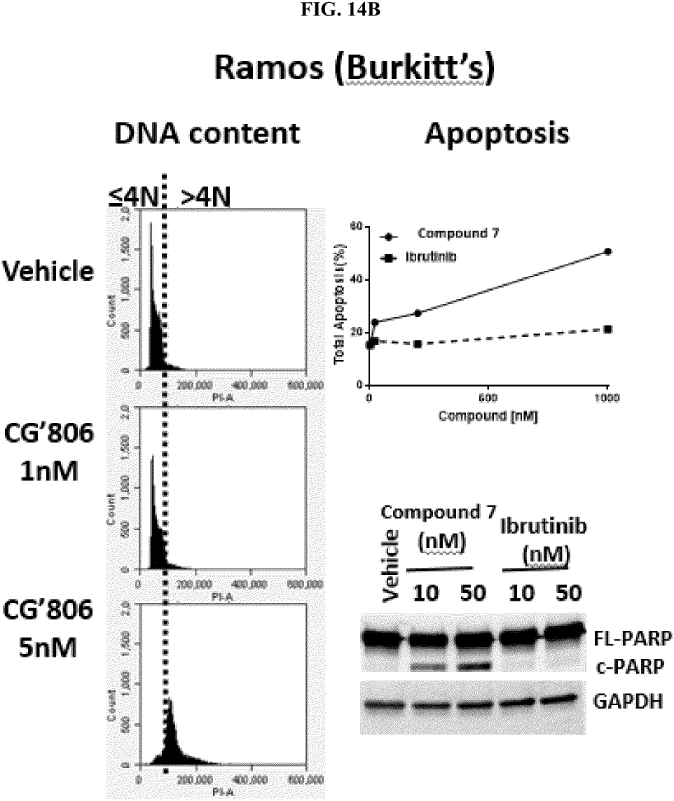

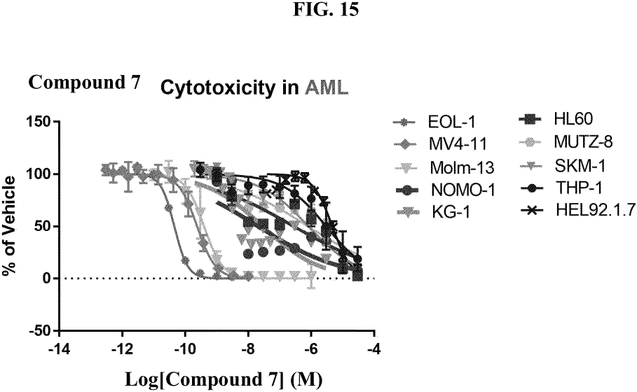

Mino and Ramos cell lines were treated with increasing concentrations of Compound 7 or ibrutinib to gauge the comparative effect that Compound 7 has on inducing polyploidy in B-cell malignant cell lines relative to ibrutinib. As shown in FIGS. 14A - 14B, Compound 7 effectively induced polyploidy (>4n) followed by apoptosis against Mino cells at concentrations of 0.1 and 1.0 nM, and against Ramos at a concentration of 5 nM and as indicated in the Western blot showing signatures of cell death.

### Example 21: Compound 7 Interferes With Cell Cycle Progression in B-Cell Malignancies

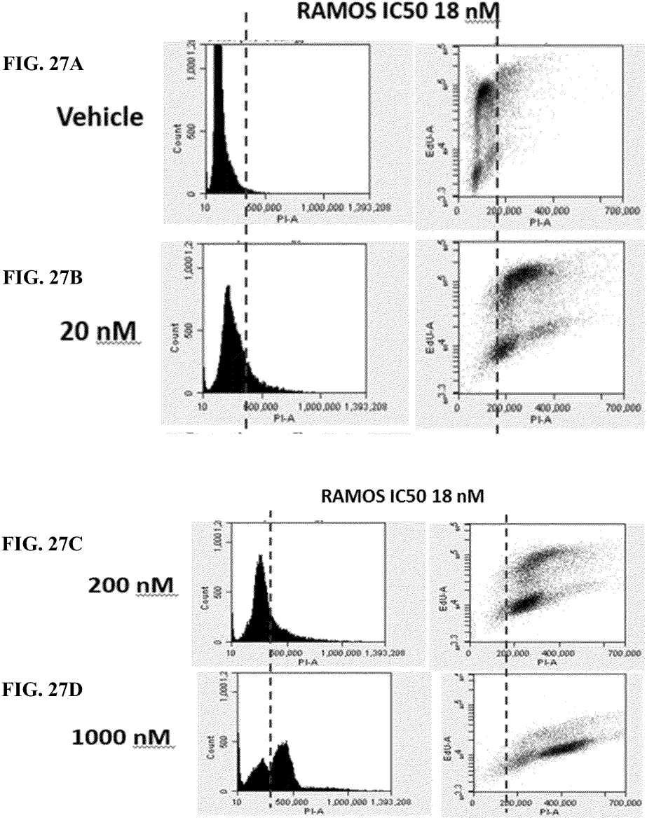

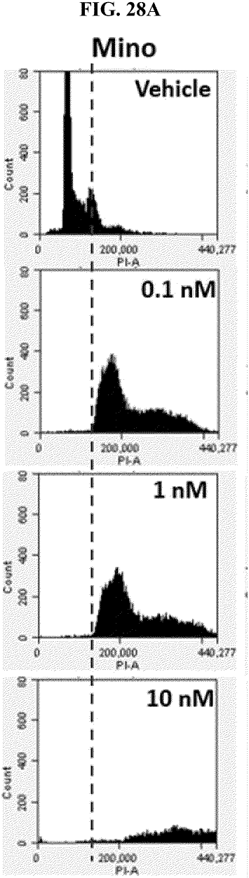

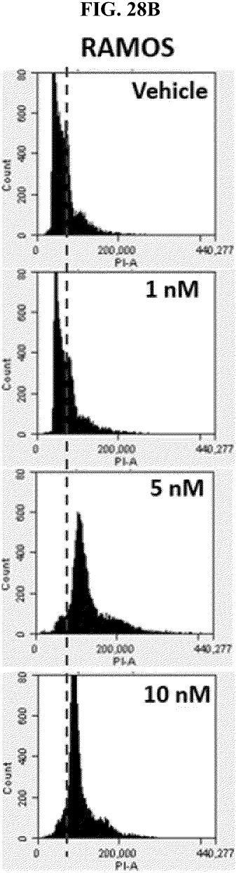

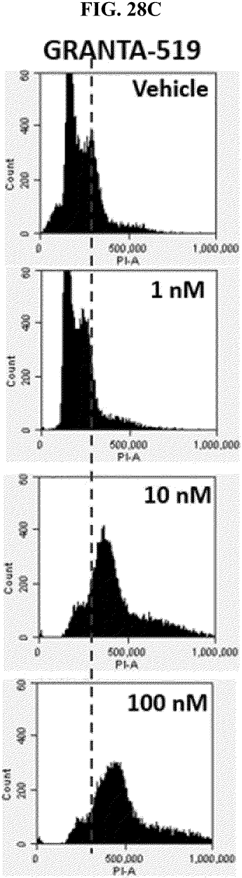

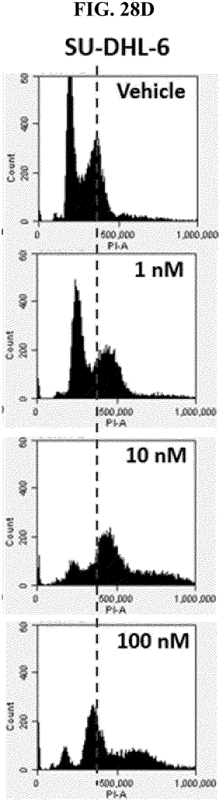

FIGS. 27A -27D and FIGS. 28A - 28D show that Compound 7 interferes with cell cycle progression. Without being bound by any theory these figures show that Compound 7 interferes with the cell signaling pathways in B-cell malignant cell lines. The data presented in FIGS. 27A -27D and FIGS. 28A - 28D were generated using well-known protocols for measuring cell cycle progression (e.g., EdU and/or PI staining followed by FACS analysis using the BD Accuri C6 flow), and is readily apparent to a skilled artisan.

### Example 22. Compound 7’s Inhibitory Effects on Cell Signaling in B-Cell Malignant Cell Lines

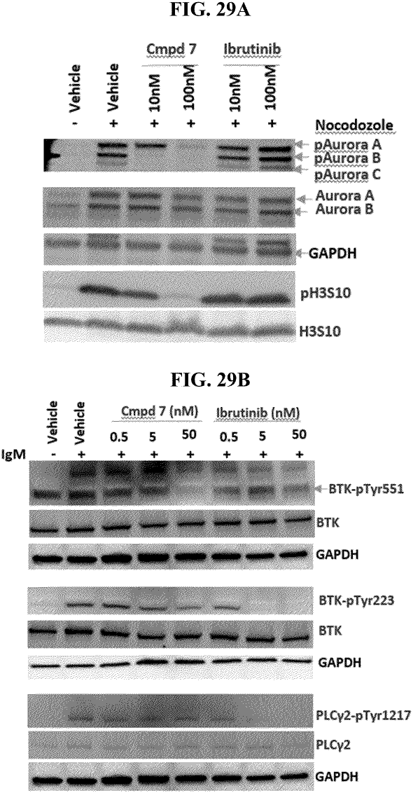

FIGS. 29A - 29B show that Compound 7, relative to ibrutinib, inhibits BTK and Aurora kinase activity in Ramos cells.

FIGS. 30A - 30B show that Compound 7 affects the BCR signaling in Ramos cells. In this experiment, Ramos cells were treated with or without Compound 7 or Ibrutinin at the indicated concentration for 1 (6 replicates) or 6 (3 replicates) hours, then stimulated with 12 µg/mL IgM for 3 min.

Without being bound by any theory these figures show that Compound 7 acts as a strong inhibitor of various cell signaling pathways in B-cell malignant cell lines.

### Example 23. Compound 7 is Cytotoxic in High Serum Conditions

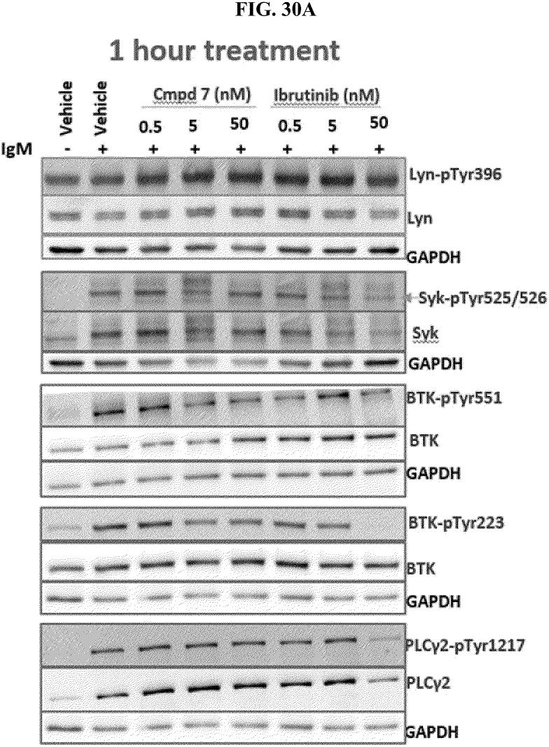

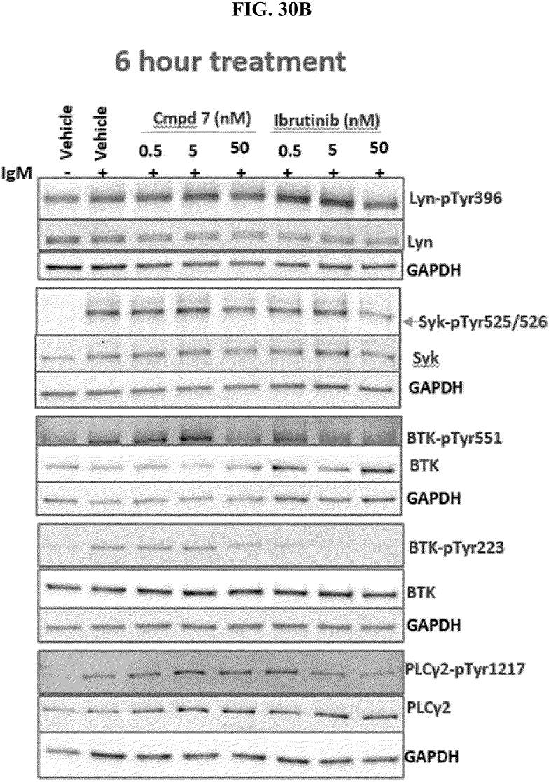

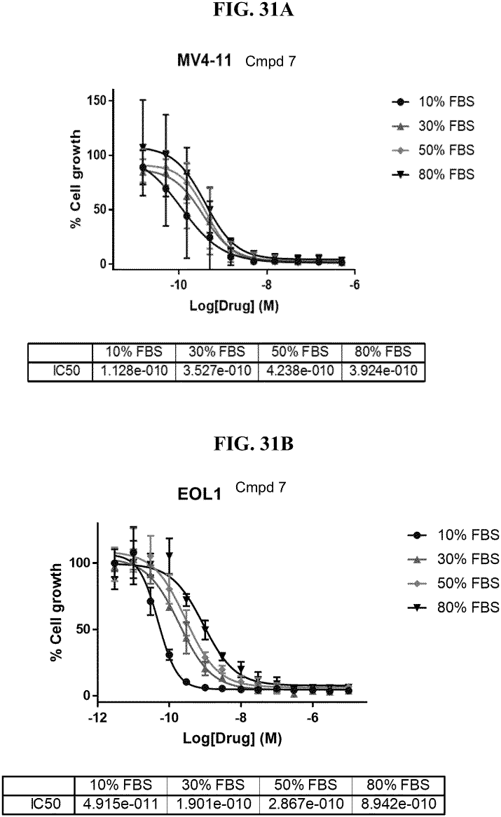

Compound 7 was tested in a dose-dependent manner at different serum concentrations. FIGS. 31A - 31B show that Compound 7 retains high activity at high serum concentration. In this experiment, MV4-11 (n=6) and EOL-1(n=3~6) cells were treated with or without Compound 7 for 72 hours in medium containing normal (10%) or high serum (30%, 50%, 80%) FBS with an MTS assay serving as an end point.

### Example 24. General Experimental Procedures

IC₅₀S and EC₅₀S: Certain cell viability studies described herein were assessed using the Tryptan blue dye exclusion method or the MTS assay. Certain apoptosis studies and related studies described herein were determined via FACS by annexin V positivity. The 50% inhibitory concentration (IC₅₀) for cell growth inhibition and the 50% effective concentration (EC₅₀) for apoptosis induction were calculated using CalcuSyn (BioSoft, Cambridge, UK).

Immunoblot assays: Cells were treated with Compound 7 at various concentrations and collected for cell lysates. The total and phosphorylated levels of the indicated proteins were determined by Western Blot.

Animal Study: Balb/c mice were injected (SQ) with human cells (e.g., FLTs-ITD-mutated leukemia cells MV4-11), and treated orally (q.d.) with the indicated doses of Compound 7 for 14 days. The effect (e.g., anti-leukemia) was assessed by measuring tumor burden. Oral toxicity was evaluated, for example, by measuring body weight. Compound 7 concentrations in plasma were measured at the indicated time points after dosing at the first day.

MTS assay based on anti-proliferation assay: MTS assay was performed to evaluate the anti-proliferative extracellular signal-regulated kinase (Barltrop, J. A. et al., (1991) 5-(3-carboxymethoxyphenyl)-2-( 4,5-dimethylthiazoly)-3-( 4-sulfophenyl) tetrazolium, inner salt (MTS) and related analog of3-( 4,5-dimethylthiazolyl)-2,5,-diphenyltetrazolium bromide (MTT) reducing to purple water soluble activities of the inventive compounds via inhibition on formazans as cell-viability indicators. Bioorg. Med. Chem. Lett. 1, 611-4; Cory, A.H. et al., (1991); Use of an aqueous soluble tertrazolium/formazan assay for cell growth assays in culture. Cancer Comm. 3, 207-12). Human lymphoma cell lines, for example, Jeko-1 (ATCC), Mino (ATCC), H9 (Korean Cell Line Bank) and SR (ATCC), and human leukemia cell lines, for example, MV4-11 (ATCC), Molm-13 (DSMZ) and Ku812 (ATCC) were used for the test according to the procedure shown below. Each of cells (e.g., Jeko-1, Mino, H9, SR, MV4-11, Molm-13 and Ku812 cells) were transferred into 96-well plates containing RPMI1640 medium (GIBCO, Invitrogen) supplemented with 10% FBS at a density of 10,000 cells/well, and then incubated for 24 hours under conditions of 37° C. and 5% 20 CO₂ . The wells were treated with each of 0.2, 1, 5, 25 and 100 µM, of the test compounds. The well was treated with DMSO in an amount of 0.08 wt%, which is the same amount as in the test compounds, which was used as a control. The resulting cells were incubated for 48 hours. MTS assays are commercially available and include the Promega CellTiter 96® Aqueous Non-Radioactive Cell Proliferation Assay. MTS assays were performed in order to evaluate cell viability of the test compounds. 20 µL of a mixed solution of 3-(4,5-dimethylthiazol-2-yl)-5-(3-carboxymethoxyphenyl )-2-( 4-sulfopheny 1)-2H-tetrazolium, inner salt (“MTS”) and phenazine methosulfate (PMS) was added to each well, and then incubated for 2 hours at 37° C. Then absorbance of the samples was read at 490 nm. The anti-proliferation activity level was calculated based on absorbance of the test compounds against that of the untreated control group. The EC50 (µM) values, in which test compounds reduce the growth of cancer cells by 50% were calculated. An assay for anti-proliferation activity was conducted by using, for example, Jeko-1, Mino, H9 and SR lymphoma cells so as to evaluate the effectiveness of the inventive compounds as an anti-inflammatory agent as well as an anti-cancer agent.

RBC HotSpot Kinase Assay Protocol:

Reagents used: Base Reaction buffer; 20 mM Hepes (pH 7.5), 10 mM MgCl2, 1 mM EGTA, 0.02% Brij35, 0.02 mg/ml BSA, 0.1 mM Na₃ VO₄, 2 mM DTT, 1% DMSO. Required cofactors are added individually to each kinase reaction.

Compound 7 was dissolved in 100% DMSO to a specified concentration. The serial dilution was conducted by epMotion 5070 in DMSO. The substrate was freshly prepared in Reaction Buffer and any required cofactors to the substrate solution were added. The kinase was added to the solution and gently mixed. Compound 7 was added in 100% DMSO into the kinase reaction mixture by Acoustic technology (Echo550; nanoliter range), and incubated for 20 min at room temperature. ³³P-ATP (Specific activity 10 µCi/µl) was added into the reaction mixture to initiate the reaction, which was incubated for 2 hours, whereupon kinase activity was detected by the filter-binding method.

Signaling assay. Cells (e.g., MV4-11) were treated with a specified dose (e.g., at 500 pM) Compound 7 or a comparative drugs (e.g., quizartinib), or vehicle (e.g., DMSO) and then subjected to Western Blot on FLT3 and its downstream signals.

Cytotoxicity procedure: Cells were seeded in 96-well plate and treated with vehicle DMSO or Compound 7 at a specified concentration and incubated for 72 hours. At the end of 72 hour incubation period, MTS-based assay was performed and IC50s were determined by GraphPad Prism7.0.

Cell-cycle analysis: Cells were treated with vehicle, DMSO or Compound 7 and were stained with PI and EdU, and then analyzed by flow cytometry to determine the phases of cell cycle.

The publications discussed herein are provided solely for their disclosure prior to the filing date of the present application. Nothing herein is to be construed as an admission that the present invention is not entitled to antedate such publication by virtue of prior invention.

All publications, patents and patent applications, including any drawings and appendices therein are incorporated by reference in their entirety for all purposes to the same extent as if each individual publication, patent or patent application, drawing, or appendix was specifically and individually indicated to be incorporated by reference in its entirety for all purposes.

While the invention has been described in connection with proposed specific embodiments thereof, it will be understood that it is capable of further modifications and this application is intended to cover any variations, uses, or adaptations of the invention following, in general, the principles of the invention and including such departures from the present disclosure as come within known or customary practice within the art to which the invention pertains and as may be applied to the essential features hereinbefore set forth and as follows in the scope of the appended claims.

1-147. (canceled)

148. A method of inhibiting or reducing mutated Fms-related tyrosine kinase 3 (FLT3) activity or expression in a subject, comprising administering Compound 7: 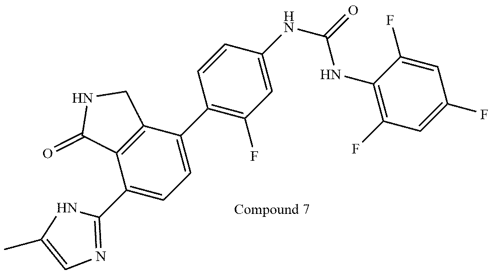
or a pharmaceutically acceptable salt thereof, wherein:
i) the FLT3 mutation comprises at least one point mutation on one or more residues selected from the group consisting of D835, R834, F691, K663, N841 and Y842; or
ii) wherein the FLT3 mutation comprises at least one point mutation on one or more residues selected from the group consisting of D835, R834, F691, K663, N841 and Y842 and an internal tandem duplication (ITD) mutation.

149. The method of claim 148, wherein the mutated FLT3 comprises at least one mutation at D835.

150. The method of claim 148, wherein the mutated FLT3 comprises at least one mutation at F691.

151. The method of claim 148, wherein mutated FLT3 further comprises at least one point mutation in the tyrosine kinase domain of FLT3.

152. The method of claim 148, wherein the mutated FLT3 further comprises at least one point mutation in the activation loop of FLT3.

153. The method of claim 148, wherein the mutated FLT3 has one or more mutations selected from the group consisting of FLT3-D835H, FLT3-D835V, FLT3-D835Y, FLT3-ITD-D835V, FLT3-ITD-D835Y, FLT3-ITD-D835H, FLT3-F691L, FLT3-ITD-F691L, FLT3-ITD-K663Q, FLT3-ITD-N841I, FLT3-N841I, FLT-3R834Q, FLT3-ITD-834Q, FLT3-D835G, FLT3-ITD-D835G, FLT3-Y842C, and FLT3-ITD-Y842C.

154. The method of claim 148, wherein the inhibiting or reducing mutated Fms-related tyrosine kinase 3 (FLT3) activity or expression in a subject results in the treatment of cancer.

155. The method of claim 154, wherein the cancer is a hematological malignancy or B cell malignancy.

156. The method of claim 155, wherein the hematologic malignancy is leukemia.

157. The method of claim 156, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, or mantle cell lymphoma.

158. A method of treating a hematologic malignancy associated with a mutated FLT3 in a subject in need thereof, comprising administering a Compound 7: 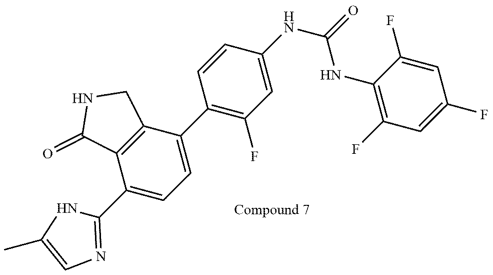
or a pharmaceutically acceptable salt thereof, wherein the subject shows resistance or relapse to an inhibitor of FLT3 activity or expression, wherein:
i) the FLT3 mutation comprises at least one point mutation on one or more residues selected from the group consisting of D835, R834, F691, and Y842; or
ii) wherein the FLT3 mutation comprises at least one point mutation on one or more residues selected from the group consisting of D835, R834, F691, and Y842 and an internal tandem duplication (ITD) mutation.

159. The method of claim 158, wherein the FLT3 mutation comprises at least one point mutation on one or more residues selected from the group consisting of D835, R834, F691, and Y842, and an internal tandem duplication (ITD) mutation.

160. The method of claim 158, wherein the inhibitor is quizartinib, gilteritinib, sunitinib, sorafenib, midostaurin, lestaurtinib, crenolanib, PLX3397, PLX3623, crenolanib, ponatinib, or pacritinib.

161. The method of claim 158, wherein the inhibitor is quizartinib or gilteritinib.

162. The method of claim 158, wherein the hematologic malignancy is leukemia.

163. The method of claim 162, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cellacute lymphocytic leukemia, acute myeloid leukemia with trilineage myelodysplasia, mixed lineage leukemia, eosinophilic leukemia, or mantle cell lymphoma.

164. A method of inhibiting or reducing mutated Fms-related tyrosine kinase 3 (FLT3) activity or expression in a subject having a FLT3 internal tandem duplication (ITD) mutation and at least one FLT3 point mutation, comprising administering Compound 7: 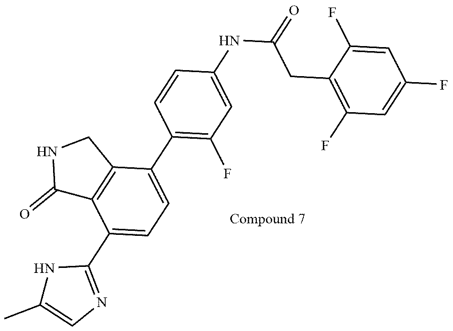
or a pharmaceutically acceptable salt thereof.

165. The method of claim 164, wherein the at least one FLT3 point mutation is on one or more residues selected from the group consisting of D835, R834, F691, K663, Y842 and N841.

166. The method of claim 164, wherein the at least one FLT3 point mutation is D835Y.

167. The method of claim 164, wherein the at least one point mutation is on one or more amino acid residue positions selected from the group consisting of 686, 687, 688, 689, 690, 691, 692, 693, 694, 695, and 696.

168. The method of claim 164, wherein the inhibiting or reducing mutated Fms-related tyrosine kinase 3 (FLT3) activity or expression in a subject results in the treatment of hematological malignancy or B cell malignancy.

169. The method of claim 164, wherein the treated B cell malignancy is selected from one or more of the group consisting of mantle cell lymphoma (MCL), B-cell acute lymphoblastic leukemia (B-ALL), Burkitt’s lymphoma, chronic lymphocytic leukemia (CLL), and diffuse large B-cell lymphoma (DLBCL).

170. The method of claim 164, wherein the hematological malignancy is leukemia.

171. The method of claim 164, wherein the leukemia is acute lymphocytic leukemia, acute myeloid leukemia, acute promyelocytic leukemia, chronic lymphocytic leukemia, chronic myeloid leukemia, chronic neutrophilic leukemia, acute undifferentiated leukemia, anaplastic large-cell lymphoma, prolymphocytic leukemia, juvenile myelomonocytic leukemia, adult T-cell acute lymphocytic leukemia, acute myeloid leukemia with trilineage myelodysplasia, mixed lineage leukemia, eosinophilic leukemia, and/or mantle cell lymphoma.
